(19) United States
(12) Patent Application Publication
(10) Pub. No.: US 2025/0194634 A1
(43) Pub. Date: Jun. 19, 2025

(54) COMPOSITE MATERIALS COMPRISING A POLYMER SCAFFOLD AND METHODS OF MAKING AND USING

(71) Applicant: Aquacultured Foods, Inc., Chicago, IL (US)

(72) Inventors: Anne PALERMO, Chicago, IL (US); Robert SCHULTZ, Chicago, IL (US); Mary KASPER, Chicago, IL (US)

(21) Appl. No.: 18/875,033

(22) PCT Filed: Jun. 16, 2023
(86) PCT No.: PCT/US2023/025634

§ 371 Date: Dec. 13, 2024

Related U.S. Application Data
(60) Provisional application No. 63/424,115, filed on Nov. 9, 2022.
(60) Provisional application No. 63/420,011, filed on Oct. 27, 2022.
(60) Provisional application No. 63/353,529, filed on Jun. 17, 2022.

(51) Int. Cl. A23J 3/20; A23L 13/00; C12N 1/16; C12N 1/20; C12N 1/34; C12N 5/00

(52) U.S. Cl. CPC A23J 3/20; A23L 13/00; C12N 1/16; C12N 1/20; C12N 1/34; C12N 5/0062; C12N 2533/40; C12N 2533/54; C12N 2533/72; C12N 2533/74; C12N 2533/76; C12N 2533/90

(57) ABSTRACT

Disclosed herein are methods and compositions for manufacturing, processing and storing composite materials, containing bacterial cellulose and eukaryotic cells, e.g., fungal cells. The methods comprise a first culture, in which bacteria produce a cellulose-containing scaffold, and a second culture, in which the scaffold is cultured in the presence of eukaryotic cells, such as fungi or animal cells, which populate the scaffold to produce the composite material.

### REFERENCE TO RELATED APPLICATIONS

This application claims the benefit of the priority dates of U.S. Provisional application No. 63/353,529, filed on Jun. 17, 2022; U.S. Provisional application No. 63/420,011, filed on Oct. 27, 2022 and U.S. Provisional application No. 63/424,115, filed on Nov. 9, 2022; the contents of all of which are incorporated herein by reference in their entirety.

### STATEMENT AS TO FEDERALLY SPONSORED RESEARCH

None.

### BACKGROUND

The food industry is developing food products that simulate meat from animals, but that do not include ingredients from killed animals. Rather, these products are often made from microorganisms, or combinations of microorganisms. Such products include, for example, artificial beef, chicken, pork and fish. One aim of these efforts is to provide an attractive, palatable product, particularly products that mimic the taste, mouthfeel and nutrition of the animal-based products they are intended to mimic.

### SUMMARY

This disclosure relates, in part to the manufacture and processing of composite materials comprising a polymer scaffold (e.g., bacterial cellulose, a chitosan-alginate hydrogel, an alginate-gelatin polymer, cellulose acetate fibers, cellulose acetate-chitosan polymer, an agarose hydrogel, or an agarose-alginate hydrogel) and, optionally, various types of eukaryotic cells. Such composite materials can be used, for example, as food products, and for various medical applications, such as restorative or reconstructive surgery.

Disclosed herein are methods for manufacture and use of a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel), in which bacteria are cultured to produce a cellulose-containing extracellular matrix, followed by removal of bacterial cells from the culture to produce a decellularized scaffold. Eucaryotic cells can be grown on and within the decellularized scaffold, under culture conditions favorable to the type of eukaryotic cell being grown on the scaffold, to produce various types of composite materials useful as foodstuffs and medical materials.

Existing methods for manufacturing composite materials for use as food products and as medical materials include, interalia, the co-culturing of microorganisms such as, for example, bacteria and fungi. In such methods, two different microorganisms (e.g., a bacterium and a fungus) are grown in the same culture medium. See, for example, International Publication WO 2022/245683; the disclosure of which is incorporated herein by reference in its entirety, for the purposes of describing methods and compositions for making composite materials.

The present disclosure provides, e.g., methods and compositions in which composite materials for use as, e.g., food products, are manufactured in a two-step process. In a first step (first culture), bacteria are cultured in a liquid culture medium under conditions in which they produce a cellulose-containing extracellular matrix (ECM), forming a scaffold. The culture medium can contain defined components e.g. a carbon source, a nitrogen source in nutrients. In a second step, the cellulose-containing bacterial ECM, separated from the bacterial culture, is placed in a liquid culture in which eukaryotic cells are grown. In this second culture, the bacterial ECM provides a scaffold for growth of the eukaryotic cells. The product of the second culture is a composite material containing protein produced by the eukaryotic cell within a cellulose—containing bacterial scaffold and, optionally, single cell protein. Single cell protein refers to protein derived from cells of microorganisms, such as fungi.

Alternatively, the product can be made using a one-step culture process in which one or more bacteria and one or more fungi are grown in coculture to produce a pellicle comprising a fungal protein. This pellicle can be processed according to the method provided herein.

For the manufacture of food products, use of the two-step system disclosed herein makes it possible for the manufacturer to adjust the conditions of the bacterial culture to optimize the desired properties of the scaffold, which will affect the texture and mouth-feel of the food product. Conditions for eukaryotic cell culture can be separately adjusted to optimize production of desired eukaryotic proteins, which will affect the flavor of the food product.

For the manufacture of medical materials, conditions of the bacterial culture can be adjusted to produce a cellulose matrix having desired properties such as strength and elasticity; while conditions of the eukaryotic cell culture can be adjusted to optimize properties relating to drug delivery, antibacterial action and wound healing.

In additional embodiments, a first, bacterial, culture contains one or more emulsifiers, such that the first culture is a foam, rather than a liquid culture; resulting in the production of a foamed scaffold. Having the scaffold in the form of a foam allows even greater control of the physical properties (e.g., texture) of the composite material.

In further embodiments for the manufacture of food products, a composite material containing a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel) and eukaryotic protein(s) undergoes processing (e.g., shaping, cutting, tenderizing, emulsifying, marinating, grinding, layering, coloring) to modulate its properties; e.g., flavor, texture, color, shape and nutritional content. For example, the product can be marinated in a solution comprising components to provide slave or, e.g. a fish flavor, and/or color, e.g., a reddish color for tuna or a pinkish color for salmon.

In further embodiments, the product is free or essentially free of unwanted materials. A product is essentially free of a substance if the substance is present only in trace amounts, e.g., less than 2% by weight, less than 1% by weight, less than 0.5% by weight, or less than 0.1% by weight.

### BRIEF DESCRIPTION OF THE DRAWINGS

FIGS. 1A-1N depict exemplary food shapes made from composite materials. These include: shrimp (1A), scallop (1B), salmon steak (1C), calamari ring (1D), lobster claw (1E), crab claw (1F), calamari mantle (1G), sushi shapes (1H, 1I, 1J), nugget (1K), patty (1L), stick (e.g., fish stick) (1M), and bacon (1N).

FIG. 2 depicts marinated composite material.

FIG. 3 depicts a layered composite material (raw).

FIG. 4 depicts a layered composite material that has been sauteed.

FIG. 5 depicts comparisons of the storage moduli (in Pascals), at different strain percentages, of (a) composite material made according to the method of Example 4 (top curve, triangles), (b) calamari (middle curve, circles) and (c) tuna (bottom curve, squares).

FIG. 6 depicts comparisons of the loss moduli (in Pascals), at different strain percentages, of (a) composite material made according to the method of Example 4 (top curve, triangles), (b) calamari (middle curve, circles) and (c) tuna (bottom curve, squares).

FIG. 7 depicts scanning electron micrographs of muscle tissue from (left to right): calamari, shrimp, cod, salmon, tuna, and bacterial cellulose scaffold (BC). The top row shows representative micrographs of muscle fascicles; the bottom row shows representative micrographs of muscle fibers.

FIG. 8 depicts results of measurement of muscle fascicle diameters from (left to right): squid, shrimp cod, salmon, tuna and bacterial cellulose (BC) scaffold.

FIG. 9 depicts results of measurement of muscle fiber diameters from (left to right): squid, shrimp cod, salmon, tuna and bacterial cellulose scaffold.

FIG. 10 is a set of histograms showing angular orientation of fascicular structures in salmon (top left), calamari (middle left), cod (bottom left), bacterial cellulose scaffold (top right), shrimp (middle right) and tuna (bottom right). Dispersion angles are shown along the ordinate, and the percentage of fibers at each angle is shown as a histogram.

FIG. 11 depicts peak directional dispersions for squid, shrimp, cod, salmon, tuna and bacterial cellulose (BC) scaffold.

FIG. 12 depicts cutting forces of the muscle fibers of different aquatic species. Black bars indicate the cutting force of raw muscle fibers; gray bars indicate the cutting force of muscle fibers that were cooked to an internal temperature of 145° F. (63° C.) for at least 15 seconds. Numerical values of cutting forces (in lbs/in²) are shown below the graph. The horizontal dotted line indicates the mean cutting force (with the shading indicating the standard deviation) of an exemplary bacterial cellulose made by the method of Example 4.

FIG. 13 depicts cutting forces (in lbs/in²) of bacterial cellulose scaffolds produced by (left to right): (a) K. xylinus strain NRRL B3780; (b) K. xylinus strain ATCC 53582; (c) K. xylinus strain ATCC 53582 grown in a medium containing a 1:1 (v:v) ratio of alginate porogen to culture medium; (d) K. xylinus strain ATCC 53582 grown in a medium containing a 2:1 (v:v) ratio of alginate porogen to culture medium; (e) K. xylinus strain NRRL B3780 foamed on sterilized water; and (f) K. xylinus strain ATCC 53582 foamed on sterilized water.

FIG. 14 depicts the effect of guar gum, added during marination, on percent water retention of composite material.

FIG. 15 depicts the effect of guar gum, added during marination, on average hardness of the composite material.

FIG. 16 depicts the effect of trehalose, added during marination, on percent water retention of composite material.

FIG. 17 depicts the effect of trehalose, added during marination, on average hardness of the composite material.

FIG. 18 depicts pore densities of squid muscle fibers and of bacterial cellulose (BC) scaffold produced by K. xylinus ATCC 53582 grown in Hestrin-Schramm media.

FIG. 19 depicts the average pore surface area of squid muscle fibers and of bacterial cellulose (BC) scaffold produced by K. xylinus ATCC 53582 grown in Hestrin-Schramm media.

FIG. 20 depicts exemplary food products of this disclosure. Shown here are tuna sashimi 2001, tuna roll 2003, shrimp dumpling 2005, ginger 2007, wasabi 2009, chopsticks 2011 and soy sauce 2013.

### DETAILED DESCRIPTION

The present disclosure provides methods and compositions for manufacturing composite materials for use as food products and medical materials. In certain embodiments the methods involve, interalia, a two-step culturing protocol. In the first step (first culture), a monoculture of bacteria is used to produce a cellulose-containing scaffold, which is isolated so as to be free, or essentially free, of bacterial cells. In the second step (second culture), a monoculture of eukaryotic cells (e.g., fish cells, fungal cells) is cultured in the presence of the isolated bacterial scaffold to form a composite material, containing a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel) and eukaryotic protein (and optionally cells), which can be used as a food product or a medical material. In additional embodiments, eukaryotic cells are cultured on a non-bacterial polymer scaffold such as, for example, chitosan, alginate-gelatin, cellulose acetate, agarose or agarose-alginate.

### I. Bacterial Culture

### A. Bacteria

Any bacterium, or combination of bacteria, that produces a cellulose-containing extracellular matrix can be used in a first culture. Exemplary cellulose-producing bacteria include, for example, species of Acetobacter, Bacillus, Bifidobacterium, Brachybacterium, Brevibacterium, Carnobacterium, Corynebacterium, Enterococcus, Gluconobacter, Gluconacetobacter, Halomonas, Komagataeibacter, Lactobacillus, Lactococcus, Leuconostoc, Macrococcus, Microbacterium, Micrococcus, Oenocuccus, Propionibacterium, Proteus, Pseudomonas, Psychrobacter, Streptococcus, Streptomyces, Tetragenococcus, Weissella and Zymomonas.

In additional embodiments the cellulose-producing bacterium is a species of Komagataeibacter such as, for example, Komagataeibacter xylinus (e.g., ATCC 53582, ATCC 700178, NRRL B3780), Komagataeibacter hansenii, and/or Komagataeibacter rhaeticus. In further embodiments, the cellulose-producing bacterium is Gluconacetobacter xylinus.

In some embodiments, the culture is free or essentially free of lactic acid bacteria. Lactic acid bacteria comprise bacteria of the order Lactobacilliales. They include the genera Lactobacillus, Leuconostoc, Pediococcus, Lactococcus, Streptococcus, Aerococcus, Camobacterium, Enterococcus, Oenococcus, Sporolactobacillus, Tetragenococcus, Vagococcus, and Weissella.

Methods for the propagation and storage of bacteria, including liquid and solid media, and storage conditions such as temperature and humidity, are known in the art.

### B. Bacterial Culture Media

For production of a cellulose scaffold (e.g., a pellicle), bacteria are grown in a defined first culture medium. The first culture medium can comprise one or more of water, one or more carbon sources, one or more nitrogen sources, and one or more additional nutrients. In certain embodiments the culture medium does not comprise coconut water.

### 1. Carbon Source

The carbon source can be present in the first culture medium at a concentration of between 1 and 100 g/L. For example, the carbon source can be present at a concentration of about any of 5 g/L, 10 g/L, 15 g/L, 20 g/L, 25 g/L, 30 g/L, 35 g/L, 40 g/L 45 g/L, 50 g/L, 60 g/L, 75 g/L or more.

The carbon source can comprise a sugar, such as one or more of glucose (dextrose), fructose, sucrose, lactose, galactose, maltose, trehalose, allulose, or maltotriose. The sugar can comprise a refined sugar, a purified sugar, or a crude sugar. For example, the carbon source can comprise glucose and fructose. The carbon source can comprise a polyol, such as glycerol, erythritol, starch hydrolysates, isomalt, lactitol, maltitol, mannitol, sorbitol, or xylitol.

The carbon source can comprise xanthan, agar, alginate, or konjac glucomannan. The carbon source can be an alcohol, such as ethanol or methanol. The carbon source can comprise honey, corn syrup or agave nectar.

In certain embodiments, an alcohol can be used as a carbon source. For example, ethanol at a concentration of 0.1% to 5% (e.g., 2%) can be used as a carbon source, optionally in conjunction with other carbon sources.

### 2. Nitrogen Source

The nitrogen source can comprise organic nitrogen or inorganic nitrogen, and can be present in the medium in an amount of between 1 g/L and 15 g/L, for example, between about any of 2.5 g/L and 7.5 g/L or any amount therebetween; i.e., 2.5 g/L, 3 g/L, 4 g/L, 5 g/L, 6 g/L, 7 g/L or 7.5 g/L. In additional embodiments the nitrogen source is present in an amount of at least 5 g/L, at least 7.5 g/L, at least 10 g/L or at least 15 g/L of culture medium. In additional embodiments, the nitrogen source is present in the second culture medium in an amount of at least 0.1% by weight, at least 0.2% by weight, at least 0.3% by weight, at least 0.4% by weight, at least 0.5% by weight, at least 0.6 by weight, at least 0.7% by weight, at least 0.8% by weight, at least 0.9% by weight, or at least 1% by weight.

An organic nitrogen source can comprise amino acids or nucleotides, or compounds comprising them, such as peptides, oligopeptides, polypeptides, oligonucleotides and nucleic acids. In addition, an organic nitrogen source can comprise any one or more of: a yeast extract, a peptone, an amino acid mixture, a hydrolyzed corn protein, a hydrolyzed soy protein, a hydrolyzed pea protein, and a corn steep liquor. Certain agricultural byproducts containing high amounts of nitrogen can also be used as organic nitrogen sources.

An inorganic nitrogen source can comprise any one or more of: a nitrate salt, a nitrite salt, an ammonium salt, a urea compound, nitrogen gas, and ammonium hydroxide.

### 3. Nutrients

Additional nutrients can comprise any one or more of: salts of magnesium (e.g., magnesium sulfate heptahydrate (MgSO₄·7H₂O)), salts of calcium (e.g., calcium sulfate (CaSO₄)), salts of copper (e.g., copper sulfate (CuSO₄)), salts of manganese (e.g., manganese sulfate (MnSO₄)), ammonium salts (e.g., ammonium sulfate ((NH₄)₂SO₄)), salts of zinc (e.g., zinc sulfate (ZnSO₄)), iron-ammonium salts (e.g., ferric ammonium sulfate (FeNH₄(SO₄)₂) and ferrous ammonium sulfate (Fe(NH₄)₂(SO₄)₂)), potassium salts (e.g., potassium hydrogen phosphate (i.e., monopotassium phosphate KH₂PO₄ or dipotassium phosphate K₂HPO₄)), sodium phosphates (i.e., Na₃PO₄, Na₂HPO₄, NaH₂PO₄), ammonium nitrate (NH₄NO₃), biotin, acetic acid and sodium acetate. Other salts of calcium, magnesium, iron, zinc, copper, manganese and molybdenum can also be used as nutrients. EDTA can be included to chelate metal ions.

### 4. Exemplary Bacterial Culture Media

In one embodiment, a bacterial culture medium contains:
35 g/L glucose
25 g/L fructose
2.5 g/L yeast extract
2.5 g/L peptone
5 g/L KH₂PO₄
0.1 M acetate, pH 4.6

In an additional embodiment, a bacterial culture medium contains:
35 g/L glucose
25 g/L fructose
2.5 g/L yeast extract
2.5 g/L peptone
1.35 g/L KH₂PO₄
0.75 g/L citric acid

### C. Bacterial Culture Conditions

The first culture medium is inoculated with bacteria to initiate the culture. In certain embodiments, the inoculum is a logarithmic-phase culture of bacteria comprising 1-10% wet weight of the culture. In additional embodiments, the bacterial inoculum comprises 1% or more, 2% or more, 3% or more, 4% or more, 5% or more, 6% or more, 7% or more, 8% or more, 9% or more, or 10% of wet weight of the culture. Alternatively, the culture medium can be inoculated by introducing microorganisms picked up, e.g., from an agar plate, with a sterile loop.

### 1. Culture Container

A culture container is selected to ferment a culture and produce a bacterial cellulose scaffold (e.g., in the form of a pellicle) of a desired area and thickness. The area can be large enough to allow processing of the pellicle into many pieces useful in food production. The depth of the container can be deep enough to allow the pellicle to grow to a desired thickness. Also, the depth may not be so deep that culture medium is wasted growing the pellicle. For example, a tray having dimensions of about 27 cm long and about 20 cm wide can be filled with culture medium to a depth of about 4 cm. This embodiment produces a pellicle with a surface area of about 540 cm². The volume of the culture in this configuration is 540 cm²×4 cm, or about 2160 cm³. In this case, the surface area to volume ratio is about 1:4. A useful surface to volume ratio can be between about 1:2 and about 1:6. It is further contemplated that the culture container can have a surface area of at least any of 250 cm², 500 cm², 1000 cm², 5000 cm², 1 M², 10 M², 25 M², or 100 M².

2. pH

The pH of the medium can be adjusted if desired using, for example, phosphate, carbonate, tris, acetate or citrate, HCl, acetic acid, imidazole, bicarbonate or NaOH. A bacterial culture typically will begin with a pH between about 6 and 7.5. However, the pH can initially be adjusted in the culture to an acidic pH, e.g., between about 4 and 5.5, e.g., about 4.5 to about 4.8, e.g., about pH 5.2 or about pH 4.7. Growth of bacteria will acidify the culture, such that it reaches a pH between about pH 2 to about pH 5. In certain embodiments, the culture, after initial growth, is maintained a pH of the culture medium is maintained, e.g., through buffering, at between about pH 2 to about pH 6, e.g., between pH 2 to about pH 5.

### 3. Temperature

Bacterial cultures are conducted at a temperature suitable for production of the desired cellulose-containing scaffold by the bacteria in the culture. Culture can be conducted at a temperature between 4° C. and 50° C., or between 20° C. and 40° C., or between 20° C. and 30° C., or between 25° C. and 35° C., or between 27° C. and 32° C., or between 27° C. and 28° C., or at about 27° C., or at 27.5° C.

### 4. Humidity

The humidity of the environment in which the culture is contained contributes to the moistness of the final product. Accordingly, the relative humidity (RH) of the culture can be controlled to optimize production of a suitable scaffold. For example, bacterial culture can be conducted at a relative humidity of between 10% and 90%, or between 20% and 80%, or between 30% and 70%, or between 40% and 60%, or between 45% and 55%. In certain embodiments, the bacterial culture is conducted at a RH of 30-40%.

### 5. Oxygenation

The oxygen concentration of the bacterial culture can also be controlled, e.g., by sparging (e.g., bubbling) air into the culture and/or by adjusting the surface area of the culture. The dissolved oxygen concentration of the first culture can be between 2% and 20% by volume. For example, the dissolved oxygen content of the first culture medium can be about any of 5%, 6%, 7%, 8%, 9%, 10%, 11%, 12%, 13%, 14%, or 15%. In additional embodiments, the dissolved oxygen concentration of the first culture is at least 5%, at least 6%, at least 7%, at least 8%, at least 9%, at least 10%, at least 11%, at least 12%, at least 13%, at least 14%, or at least 15%. Optimal oxygen concentration depends on the species of bacterium being cultured. Increased aeration (i.e., higher dissolved oxygen) can contribute to a scaffold with a lower cutting force.

### 6. Length of Culture

The duration of the first culture can be selected to provide a scaffold with desired properties, such as thickness and texture. Accordingly, culture can be conducted for about 12 hours to 30 days or more. For example, culture can be conducted for about 1 day to about 20 days, about 1 day to about 2 days, about 4 days, for about 5 days, for about 6 days, for about 7 days, for about 8 days, for about 9 days, for about 10 days, for about 11 days, for about 12 days, for about 13 days, for about 14 days, for about 15 days, for about 16 days, for about 17 days, for about 18 days, for about 19 days or for about 20 days. In certain embodiments, bacterial culture is conducted for 2-20 days, for 4-14 days, for 4-16 days, for 4-18 days, for 10-14 days, for 4-30 days, for 5-14 days, for 7-12 days, or for 8-10 days. The culture can be conducted for about 12 days to 18 days. For extended cultures (e.g., 2 weeks or more), the culture medium can be supplemented with one or more carbon sources, nitrogen sources or the nutrients; or the culture medium can be replaced with fresh culture medium, either the same as, or different from, the original culture medium. In additional embodiments, a bacterial culture can be re-inoculated, with either the same or a different bacterium, after a period of initial culture.

Cultures can be conducted in static fashion or can be agitated, e.g., using a gyratory shaker. For static cultures, exemplary culture conditions are 27° C., 60% relative humidity, 8% dissolved oxygen and pH 4.6. Exemplary conditions for agitated culture are 27° C., pH 4.6, 0.25 volume of air per volume of liquid per minute (vvm) and 250 rpm.

Additional information regarding culture conditions, culture vessels and culture systems is provided in International Publication WO 2022/245683; the disclosure of which is incorporated by reference, in its entirety, for the purpose of providing information about methods, compositions and systems for the culture of bacteria and fungi.

### 7. Foamed Cultures

In certain embodiments, the first culture medium is in the form of a foam, rather than a liquid. In these embodiments, one or more emulsifying agents and, optionally, one or more gelling agents, are added to sterile culture medium prior to inoculation of the culture medium with bacteria.

### 8. Emulsifying Agents

An “emulsifier” or “emulsifying agent” is a surfactant molecule that decreases the surface area between two immiscible substances such that, when a mixture of the substances is subject to sheer, the mixture forms discontinuous droplets of one suspended liquid or gas phase in a second liquid or gas phase to form a uniform dispersion. Exemplary emulsifying agents include proteins and fats. Exemplary proteins for use as emulsifying agents include gelatin, casein, soy proteins, gluten, ovalbumin and albumin. Exemplary fats for use as emulsifying agents include monoglycerides, diglycerides, triglycerides, phospholipids (e.g., lecithins), polyglycerol polyricinoleate (PGPR), ammonium phosphatide (AMP), mono and di-glycerides, polysorbates and sodium stearoyllactylate. Exemplary monoglycerides include glyceryl monostearate, glyceryl monooleate, glyceryl monolaurate, and glyceryl monocaprylate. Exemplary diglycerides include glyceryl distearate, glyceryl dioleate, glyceryl dicaprylate and glyceryl dilaurate. Exemplary polysaccharides include dextran and β-glucans.

### 9. Gelling Agents

In other embodiments, the culture further comprises a gelling agent. A “gellant” or “gelling agent” is a substance that forms a semisolid or solid colloidal mixture when dissolved in a liquid. Gelling agents include thickening agents (e.g., gums, fibers and starches) and or hydrocolloids. Exemplary gums include xanthan gum, tragacanth gum, gum arabic, konjac, acacia, guar gum, gellan gum gelatin, pectin, locust bean gum, agar, sodium alginate, locust bean gum, cellulose gum, carrageenan, and a glucomannan polysaccharide gum. Exemplary fibers include methylcellulose and carboxymethylcellulose. Exemplary starches include maize starch, potato starch and tapioca. Starches can be native or modified by, for example, thermal modification, hydrolysis (e.g., acid-catalyzed hydrolysis, alkaline hydrolysis, enzymatic hydrolysis, dextrinization), etherification (e.g., methylation, hydroxymethylation, hydroxypropylation, carboxymethylation), esterification (e.g., acetylation, fatty acylation, phosphorylation, succinylation), cationization, cross-linking (e.g., using dicarboxylic acids, or alkaline crosslinking) or a combination of one or more of the foregoing techniques. Exemplary hydrocolloids include methylcellulose, carboxymethylcellulose, and hydroxypropyl methylcellulose.

10. Cultures with Emulsifying Agents and/or Gelling Agents

The inoculum can comprise, e.g., 1-10% of the volume of the culture. After addition of the one or more emulsifier(s) and the bacterial inoculum, the culture medium is subjected to a shear force to generate a foam. Shear force can be applied, for example, by manual agitation, by using a blender or a homogenizer, or by passing a pressurized gas through the foamed medium. In one embodiment, shear force is applied using a homogenizer at a speed of about 1,000 rpm.

A foamed culture can comprise an open-cell foam or a closed-cell foam. An open cell foam is one in which the gaseous portions of the foam (the cells) are in communication with one another; while a closed-cell foam is one which comprises a plurality of individual gas bubbles (cells) that are separate from one another and are not interconnected.

It is possible to generate foams with different degrees of overrun, depending on the nature and concentration of the emulsifiers, and the amount and duration of the shear force applied. Overrun is the volume of foam compared to the volume of non-foamed media, expressed as a percentage. It can also be expressed as the amount of gaseous material contained in the foam. Typically, a foamed bacterial culture has an overrun of 100-400%, or 150-300%, or 200-250%.

Porosity is the ratio of the void volume (i.e., the volume occupied by gas) of a foam to its total volume. Porosity of a foam can be modulated by, for example, the nature and concentrations of the emulsifiers, degree and/or duration of shear force, and temperature. In certain embodiments, the porosity of a foamed culture is between 1% and 75%, or between 1% and 50%, or between 10% and 50%, or between 20% and 50%, or between 30% and 50%, or between 40% and 50%. In other embodiments, the porosity of a foam is at least 5%, at least 10%, at least 20%, at least 30%, at least 35%, at least 40% or at least 45%.

The concentration of an emulsifier is chosen depending on the desired properties of the scaffold, and on the properties of the resulting composite material that are desired. For example, an emulsifier can be included at a concentration of 0.1% to 5% (w/v) of the culture volume. In certain embodiments, an emulsifier is present at between 0.2-2.0% (w/v), or between 0.5-1.5% (w/v) or at 1% (w/v) of the culture volume.

In certain embodiments, a foamed bacterial culture medium contains any of the components described above (or any combination thereof; e.g., the medium shown in Table 1) supplemented with the following emulsifiers:
0.2-2.0% (w/w) xanthan gum
0.2-2.0% (w/w) sodium alginate
0.2-2.0% (w/w) locust bean gum
0.2-2.0% (w/w) carrageenan
0.2-2.0% (w/w) guar gum
0.2-2.0% (w/w) glyceryl monooleate or glycerol monostearate
0.2-2.0% (w/w) glycerol distearate, or glyceryl dioleate, or glyceryl dicaprylate.

In other embodiments, a foamed bacterial culture medium contains any of the components described above (or any combination thereof; e.g., the medium shown in Table 1) supplemented with the following emulsifiers:
0.2-2.0% (w/w) xanthan gum
0.2-2.0% (w/w) gelatin
0.2-2.0% (w/w) locust bean gum
0.2-2.0% (w/w) cellulose gum
0.2-2.0% (w/w) guar gum
0.2-2.0% (w/w) whey protein concentrate

In additional embodiments, a foamed culture can contain:
0.5% (w/v) xanthan gum
1.5% (w/v) glycerol monostearate
98% bacterial culture medium (composition of bacterial culture media are described elsewhere herein).
   D. Introduction of Voids into Bacterial Cellulose Scaffold

Certain composite materials described herein comprise eukaryotic cellular material in a scaffold of bacterial cellulose, e.g., eukaryotic cells grown on a scaffold of bacterial cellulose. Scaffolds are frameworks capable of supporting other objects or materials. Scaffolds typically define voids within the scaffold which can be populated with other objects or materials. The material that forms the scaffold can be referred to as matrix. In certain embodiments, the bacterial cellulose scaffold contains voids (e.g., channels, passages, pores, pits, cavities) and the eukaryotic cells grow within those voids.

Void space in the material is useful to provide room for the living and breathing space for the additional organism/s introduced in the process and post harvest processes. Furthermore, increased surface area allows the introduction of compounds/molecules that impart sensory values, such as taste and smell.

Foaming a bacterial culture, as described above, is one way to produce a cellulose scaffold containing voids. The voids can have a size aspect, e.g., diameter, of between about 0.025 microns to about 3.0 microns.

Voids can also be introduced into a scaffold of bacterial cellulose by methods other than foaming. For example, after it is produced, a bacterial cellulose scaffold can be pricked with a needle or cut with a knife or other sharp instrument. Alternatively, voids can be introduced enzymatically, either during or after the bacterial culture, using carbohydrate-active enzymes such as cellulases and xylanases.

Prior to bacterial culture, or during the course of bacterial culture, one or more porogens can be included in the culture medium. A porogen is a solid material of specified shape and size (e.g., a sphere), that is incorporated into the nascent cellulose produced during culture. Subsequent to culture, the porogen can be dissolved away, leaving voids in the cellulose. Exemplary porogens include polysaccharide beads (e.g., alginate porogens such as sodium alginate, agar porogens), NaCl crystals and paraffin beads. A porogen can be dissolved away by, for example, heat treatment or treatment with citric acid.

### E. Isolation and Properties of Scaffold

The bacterial cultures described above (foamed and unfoamed) produce a cellulose-containing extracellular matrix (scaffold), that contains bacterial cells and may also contain bacterial protein(s) depending on the culture conditions. The cellulose can be in the form of nanocellulose microfibrils. In certain embodiments, the scaffold forms a pellicle on the surface of a first culture medium.

Depending on culture conditions and duration, the scaffold produced by the first culture can have a thickness of between 1 mm and 10 cm. For example, the scaffold can have a thickness of at least any of about any of 1 mm, 2 mm, 5 mm, 10 mm, 20 mm, 30 mm, 40 mm, 50 mm, or any integral value between 1 mm and 100 mm. The scaffold can have a thickness between, for example, any of 1 mm to 5 mm, 5 mm to 10 mm, 2 mm to 6 mm, 3 mm to 7 mm, 4 mm to 8 mm, or 5 mm to 9 mm.

The thickness of the pellicle is a function of the length of time of incubation and the amount of carbon, nitrogen and other nutrients in the culture medium. The longer the culture is incubated, the thicker the pellicle will become. Growing the pellicle further depends on the presence of nutrients to feed the bacteria producing the pellicle. For certain uses, a pellicle can be as little as 2.5 mm thick. In the case of artificial fish products, such as for use in producing sushi, the pellicle can be grown to a thickness of about 3.5 cm, which is a typical size of a block of fish used by sushi chefs. Sushi blocks can be around 1 inch (2.5 centimeters) wide, 2 inches (5 centimeters) long, and 0.75 inches (2 centimeters) thick. However, the dimensions can vary. Accordingly, composite materials provided herein can have a volume between about 15 cm³ (about 2.5 cm×2.5 cm×2.5 cm) and about 25 cm³ (2.5 cm×5 cm×2 cm), or between about 15 cm³ and 125 cm³.

### 1. Isolation

In certain embodiments, the scaffold produced by the bacterial culture is isolated from the first culture medium and decellularized. A decellularized scaffold is free or essentially free of living or dead (i.e., cells with intact cell membranes, but not metabolizing) bacterial cells. A scaffold is essentially free of living or dead cells if the cells make up no more than any of 5%, 4%, 3%, 2%, 1%, 0.5%, or 0.1% of the mass of the scaffold material. Accordingly, the scaffold can be isolated by removing it from the first culture medium and removing the bacterial cells from the scaffold.

To this end, the scaffold (e.g., in the form of a pellicle) is removed from the culture vessel and washed to remove any bacterial cells associated with the scaffold. For example, the scaffold is removed from the first culture and washed with an alkaline solution (e.g., NaOH) at elevated temperature. In certain embodiments, the isolated scaffold is washed with 0.1%-5% (w/v) NaOH, or from 0.2%-4% (w/v) NaOH, or from 0.5%-2% (w/v) NaOH or is washed with 1% (w/v) NaOH. An alkaline environment disrupts the cell wall and cell membranes, killing living organisms and sterilizing the biomaterial. The wash temperature is between 50-100° C., or between 55-95° C. or between 60-95° C. or is 90° C. Wash time can be about any of 5, 10, 20, 30, 40, 50 or 60 min, or any value within the range of 1-60 minutes. In one embodiment, the scaffold is washed with 1% (w/v) NaOH at 90° C. for 30 minutes; after which the scaffold is washed one or more times (e.g., twice) with distilled water. In certain embodiments, subsequent to its decellularization by washing in alkali, the scaffold can be boiled (e.g., at least 90° C.) in sterile water until the rinse water has a pH of about 7.

Washing the scaffold with NaOH serves, inter alia, to remove bacterial cells, thereby providing an isolated scaffold that is free, or essentially free, of bacterial cells, e.g., intact cells. A scaffold that is “essentially free” of bacterial cells is one in which bacteria comprise less than 5% of the weight of the isolated scaffold. In certain embodiments, bacteria comprise less than 4%, or less than 3%, or less than 2.5%, or less than 2%, or less than 1.5%, or less than 1%, or less than 0.75%, or less than 0.5%, or less than 0.25%, or less than 0.2%, or less than 0.1%, or less than 0.05%, or less than 0.02%, or less than 0.01%, or less than 0.005%, or less than 0.001% of the weight of a scaffold that is “essentially free” of bacteria.

A scaffold can also be decellularized (i.e., rendered free or essentially free of bacteria) by extraction with a detergent, for example, an ionic detergent such as sodium dodecyl sulfate, or a non-ionic detergent such as Triton X-100. If desired, the decellularized scaffold can be stored in sterile water at reduced temperature (e.g., 4° C.).

Alternatively, the pellicle can be removed from the culture medium and washed. Then, microorganisms can be killed by treatment with an acidic solution and heat. For example, the pellicle can be boiled for about 30 minutes to about one hour in an acidic solution, e.g., a solution of about 0.5 M citric acid (around pH 4.5).

Boiling the pellicle, e.g., in an acid solution, also improves tenderness of the ultimate product (e.g., reduces cutting force).

### 2. Crystallinity

Crystallinity (or the crystallinity index) is a property of cellulose that describes the relative abundance of ordered cellulose fibers, as compared to amorphous cellulose. It is expressed as the ratio of crystalline cellulose to amorphous cellulose as determined by, for example, X-ray crystallography, nuclear magnetic resonance (e.g., ¹³C NMR) and Fourier transform infrared spectroscopy. In certain embodiments, the crystallinity of the scaffold is 30-90% or 40-80% or 50-70% or 55-65%. In additional embodiments, the crystallinity of the scaffold is at least 10%, at least 20%, at least 30%, at least 40% or at least 50%.

The crystallinity of the scaffold will affect the second, e.g., fungal, culture in that the crystallinity of the scaffold is inversely related to eukaryotic cell growth. That is, a higher crystallinity will allow less proliferation of eukaryotic cells; while a lower crystallinity will allow greater fungal proliferation.

### 3. Cutting Force

Cutting force is a property that describes the resistance of a material to the intrusion of a blade. In certain embodiments, the cutting force of the scaffold is less than 5 kg, less than 4 kg, less than 3 kg, less than 2 kg or less than 1 kg. In additional embodiments, the cutting force of the scaffold is between 0.1-5 kg, 0.2-4 kg, 0.3-3 kg, 0.4-2.5 kg or 0.5-2.0 kg. The cutting force can be between 2 g and 15 g.

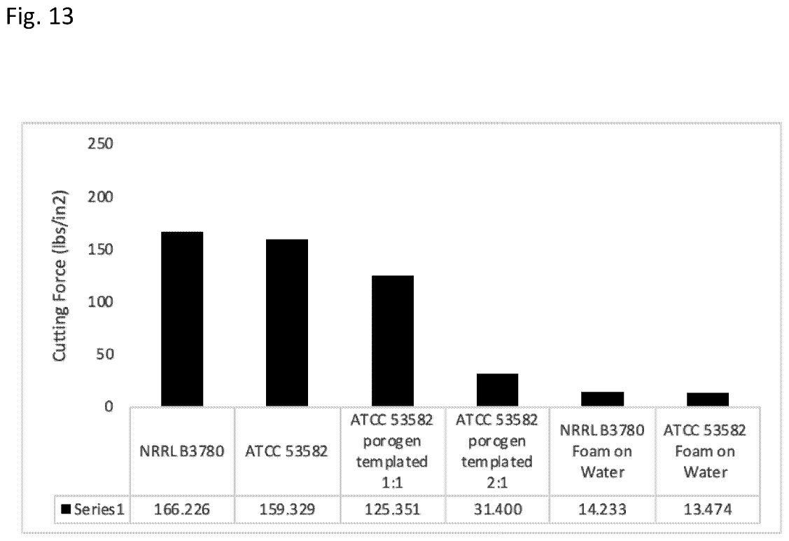

The average cutting force of seafood can range from 1.5-150 psi for raw applications and between 2-60 psi for cooked applications. For example, raw tuna has a cutting force of 1.670 psi, raw scallop has a cutting force of 1.651 psi, raw salmon has a cutting force of 6.152 psi and cooked shrimp has a cutting force of 15.147 psi. Products here can have pellicle cutting forces between 10-350 psi depending on the bacterial strain of interest (see Table 11 and FIG. 13). A wide range of achievable cutting forces is useful for mimicking various seafood applications. Specifically, K. xylinus ACF LGCY (19.475 psi) can be used to emulate raw tuna and scallop, whereas K. hansenii (32.810 psi) can be used to emulate cooked shrimp. By including porogens and/or emulsifiers in the bacterial culture, the cutting force of the bacterial cellulose scaffold can be reduced, up to 92%. In certain embodiments, the product has a cutting force in about the range of natural seafood.

### 4. Tensile Strength

Tensile strength is the maximum tension that a material can withstand, while being stretched or pulled, before breaking. In certain embodiments, the tensile strength of the scaffold is 0.05 to 5 kg, or 0.06 to 4 kg, or 0.07 to 3 kg, 0.08 to 2.5 kg, 0.09 to 2.25 kg or 0.1 to 2 kg.

Additional information relating to the production, engineering and applications of bacterial cellulose is provided in Reshmy et al. (2021) Bioengineered 12:11463-11483.

### II. Additional Polymer Scaffolds

Other materials, e.g., polymers, in addition to bacterial cellulose, can be used as a scaffold for the manufacture of composite materials with fungal, or other eukaryotic, cells. These include, for example, chitosan-containing hydrogels, alginate-containing hydrogels, gelatin-containing scaffolds, cellulose acetate-containing scaffolds, cellulose acetate chitosan-containing scaffolds, agarose-containing hydrogels, and hyaluronic acid containing scaffolds, in one or more combinations thereof.

### A. Chitosan-Containing Scaffolds

A porous chitosan alginate hydrogel is made by combining a solution of 4 wt % chitosan in acetic acid with 4 wt % alginate in acetic acid. The hybrid solution is cast into a mold shape (if desired), frozen at −20° C. for 8 hrs, critically dried by lyophilization, and crosslinked in a 0.2 M CaCl₂ solution for 10 min. See, e.g., US Patent Application Publication No. US 2012/0272347.

A chitosan-hyaluronic acid scaffold is made by combining 2-8 wt % chitosan with 1% hyaluronic acid in acetic acid. See, for example, Erickson et al. (2018) Ad. Healthc. Mater. 7:e1800295.

### B. Alginate-Gelatin Scaffold

An alginate-gelatin scaffold is constructed by combining 2.5% medium viscosity alginate and 5% gelatin; and extruding the mixture through a syringe needle with a diameter of 0.4 mm, into a solution of 1.1 wt % CaCl₂, which crosslinks the polymer. This polymer has been shown to be suitable for 3D printing; therefore it is advantageous for use in composite materials that require fine spatial resolution and shape specificity. See, for example, Chawla et al. (2020) Intl. J. Biol. Macromolecules 144:560-567.

### C. Cellulose Acetate Scaffold

To construct a cellulose acetate scaffold, 29 kDa cellulose acetate is dissolved in acetone (acetic acid can also be used as a solvent) at a concentration between 15-16.5 wt %. The polymer solution is then loaded into a dual-cylinder electrospinning apparatus in which pressure is maintained at 16 psi. A high voltage power supply (12 kV) is connected to the exterior chamber extrusion needle and grounded to an aluminum foil covered collection plate 14 cm away from the needle tip. See, for example, Rubenstein et al. (2010) J. Biomaterials Sci., Polymer Ed. 21:1713-1736.

### D. Cellulose Acetate-Chitosan Scaffold

Low molecular weight chitosan was dissolved at 0.25 wt % with 15% cellulose acetate in acetone. The solution was loaded into a dual-cylinder electrospinning apparatus where pressure was maintained at 25 psi. A high voltage power supply (14 kV) is connected to the exterior chamber extrusion needle and grounded to an aluminum foil covered collection plate 14 cm away from the needle tip. See, for example, Rubenstein et al. (2010) J. Biomaterials Sci., Polymer Ed. 21:1713-1736.

### E. Agarose Hydrogel

An agarose hydrogel is produced by heating a 1-2 wt % solution of agarose, in water, at 95° C. until the agarose is dissolved, followed by rapid cooling and gelation in an ice bath.

### F. Agarose-Alginate Hydrogel Scaffold

To construct an agarose-alginate scaffold, 1-2% ultra low melting type IX agarose is dissolved in calcium-free phosphate buffered saline at 37° C. 1.5-3% alginate is dissolved in an aqueous solution (e.g., water or 40 mM HEPES, 300 mM NaCl) at 37° C. and added to the agarose solution. Hydrogel formation is achieved by adding one-tenth volume of 102 mM CaCl₂ to the agarose-alginate solution to crosslink the alginate, followed by room temperature incubation to induce agarose gelation. See, for example, Ferrándiz et al. (2021) Biomedicines 9:834.

### III. Eukaryotic Cell Culture

In a second culture, a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel), e.g., produced by any of the methods described above, is included in a second culture of one or more eukaryotic cells, and the eukaryotic cells populate the scaffold to form a composite material containing bacterial cellulose, eukaryotic protein and, optionally, live eukaryotic cells. The second culture can be performed in a vessel or container containing the second culture medium, the scaffold and the eukaryotic cells. The composite material produced in the second culture can be used, for example, as a food product, or as a medical material.

In certain embodiments, eukaryotic cells are inoculated into a second culture medium that is in contact with (e.g., underneath) a sheet of a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel) (e.g., a pellicle); and the eucaryotic cells grow through the cellulose due to the oxygen gradient of low oxygen in the second culture medium and higher oxygen in the atmosphere above the cellulose sheet. The inoculum can be between about 0.1% and about 85% of the volume of the second culture. For example, it can be between about 1% and about 10% percent of the volume or between about 10% and about 75% of the volume. The inoculum can also be introduced by scraping a plate on which the eukaryotic cells, e.g., fungi, are growing, and introducing the scrapings into the second culture.

In additional embodiments, a sheet of comprising a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel) containing voids is produced by any of the method described above, and eukaryotic cells are inoculated onto the sheet in the presence of a second culture medium. The eukaryotic cells grow through the voids to generate a cellulose scaffold interpenetrated with cell biomass.

Eukaryotic cells that can be used in the second culture include animal cells, plant cells and fungal cells. Exemplary animal cells include vertebrate cells, chordate cells, echinoderm cells, crustacean cells and molluscan cells.

### A. Fungal Cells

Fungi are well suited for scaled food production because of their rapid rate of cell replication, aggressive digestion, colonization timing, adaptability, high protein production and ease of propagation. Many species of fungi are already accepted as safe in the human diet. Therefore, in one aspect, provided herein are food products containing fungi or fungal protein, which can be significantly higher in protein and fungal cells than are classic fermented foods that contain fungi.

Any fungus or combination of fungi can be used in the second culture. In various embodiments, the number of different fungi used in the second culture can be 1, 2, 3, 4, 5 or more than 5. Exemplary fungi include those belonging to divisions Ascomycota (e.g., Aspergillus, yeast, Fusarium, Penicillium), Chytridiomycota, Zygomycota (e.g. Rhizopus oligosporus), Basidiomycota, Deuteromycota or Glomeromycota.

In some embodiments the fungus can be any of Aspergillus (e.g., Aspergillus oryzae), Fusarium (e.g., Fusarium venenatum), tea fungus (e.g., Medusomyces gisevii Lindau), Geotrichum (e.g., Geotrichum candidum), Penicillium (e.g., Penicillium camembertior Penicillium roqueforti), Neurospora (e.g., Neurospora crassa, Neurospora intermedia), Paecilomyces (e.g., Paecilomyces variotih), Pleurotus (e.g., Pleurotus ostrealus (oyster mushroom)), Lentinula (e.g., Leninula edodes (shitake mushroom)) or Rhizopus (e.g., Rhizopus oligosporus). The fungus can be a filamentous fungus or a yeast.

Exemplary fungi (e.g., yeasts) include those that proliferate in an acidic pH environment (i.e., pH less than 6, 5.5, or 5), those that have a low flocculation rate and those that can grow within the extracellular matrix (scaffold) formed by the bacteria.

In some embodiments, the fungus is not a strong fermenter (e.g., is not Saccharomyces cerevisiae). Fungi that are not strong fermenters do not appear dark (black or purple) or green when grown on eosin methylene blue (EMB) agar.

In some embodiments, the fungus is a yeast. Exemplary yeast include Candida (e.g., Candida utilis), Rhodotorula (e.g., Rhodotorula mucilaginosa), Cyberlindnera (e.g. Cyberlindnera jadinii), Pichia (e.g., Pichia pastoris) and Saccharomyces (e.g., Saccharomyces cerevisiae).

In certain embodiments, the fungus is Aspergillus oryzae, (e.g., strain NRRL 3485).

### B. Animal Cells

Vertebrate cells include, but are not limited to, primate cells, mammalian cells, avian cells, piscine cells, reptilian cells and amphibian cells. Exemplary primate cells include human and hominid (e.g., monkey, ape) cells. Exemplary animal types include stem cells (e.g., mesenchymal stem cells), chondrocytes, chondroblasts, osteocytes, osteoblasts, tenocytes, tenoblasts, myocytes, myoblasts, myosatellite cells, adipocytes and fibroblasts.

Exemplary mammalian cells include bovine, porcine, ovine and equine cells. Exemplary avian cells include chicken, turkey, partridge, game hen, duck and goose cells.

Exemplary piscine cells include, but are not limited to tuna, salmon, yellowtail, flounder, halibut, shad, mackerel, sea bass, porgy, snapper, cod, tilapia, pollock, catfish, sardine, smelt, anchovy, eel and pangasius. In certain embodiments, piscine cells are tuna (thunnus albacares) or salmon (salmo salar).

Exemplary reptilian cells include snake and lizard.

Exemplary amphibian cells include toad, frog, salamander, newt and eft.

Exemplary echinoderm cells include sea urchin cells.

Exemplary crustacean cells include shrimp, crab, lobster, crayfish and prawn.

Exemplary molluscan cells include clam, mussel, oyster, scallop abalone, squid and octopus cells.

In certain embodiments, eukaryotic cells do not include human cells. In these embodiments, cellular material, as defined herein, does not contain a protein encoded by the human genome. In particular, and by way of example, the cellular material does not contain a human actin protein, a human myosin protein, a human troponin protein, a human actinin protein, or a human globin protein.

### C. Plant Cells

Exemplary plant cells include fruits and vegetables.

### IV. Eukaryotic Cell Culture

### A. Fungal Culture

In certain embodiments, the eukaryotic cell is a fungal cell. In the second culture, a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel), produced as described above, is included in a culture of fungal cells, and the fungal cells populate the scaffold to form a composite material containing a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel), fungal protein and, optionally, fungal cells. The second culture can be performed in a container or vessel containing the second culture medium, the scaffold and the fungal cells. The composite material produced in the second culture can be used, for example, as a food product. An “edible product” refers to a composition that can be consumed by a human or animal without toxic effects. A “food product” refers to an edible product that provides nutritional value.

In certain embodiments, fungal spores or mycelia are inoculated into a second, fungal, culture medium that is in contact with (e.g., underneath) a sheet of, e.g., bacterial cellulose (e.g., a pellicle); and fungal mycelium grows through the cellulose due to the oxygen gradient of low oxygen in the second culture medium and higher oxygen in the atmosphere above the cellulose sheet.

In additional embodiments, a sheet of a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel) containing voids is produced by any of the method described above, and fungal cells are inoculated onto the sheet in the presence of a second, fungal culture medium. The fungal mycelia grow through the voids to generate a cellulose scaffold interpenetrated with fungal biomass.

### 1. Fungal Culture Media

The fungal culture medium can comprise water, one or more carbon sources, one or more nitrogen sources and, optionally, one or more additional fungal nutrients.

### a) Carbon Source

The carbon source can be present in the first culture medium at a concentration of between 1 and 100 g/L. For example, the carbon source can be present at a concentration of 5 g/L, 10 g/L, 15 g/L, 20 g/L, 25 g/L, 30 g/L, 35 g/L, 40 g/L 45 g/L, 50 g/L, 60 g/L, 75 g/L or more.

The carbon source can comprise a sugar, such as one or more of glucose (dextrose), fructose, sucrose, lactose, galactose, maltose, trehalose, allulose, or maltotriose. Carbon source can comprise will consist essentially of glucose and fructose. The sugar can comprise a refined sugar, a purified sugar, or a crude sugar. The carbon source can comprise a polyol, such as glycerol, erythritol, starch hydrolysates, isomalt, lactitol, maltitol, mannitol, sorbitol, or xylitol. The carbon source can comprise xanthan, agar, alginate, or konjac glucomannan. The carbon source can be an alcohol, such as ethanol or methanol. The carbon source can comprise honey, corn syrup, malt extract or agave nectar.

In certain embodiments, an alcohol can be used as a carbon source. For example, ethanol at a concentration of 0.1% to 5% (e.g., 2%) can be used as a carbon source, optionally in conjunction with other carbon sources.

### b) Nitrogen Source

The nitrogen source can comprise organic nitrogen or inorganic nitrogen, and can be present in the medium in an amount of between 1 g/L and 15 g/L, for example, between 2.5 g/L and 7.5 g/L or any amount therebetween; i.e., 2.5 g/L, 3 g/L, 4 g/L, 5 g/L, 6 g/L, 7 g/L or 7.5 g/L. In additional embodiments the nitrogen source is present in an amount of at least 5 g/L, at least 7.5 g/L, at least 10 g/L or at least 15 g/L of culture medium. In additional embodiments, the nitrogen source is present in the second culture medium in an amount of at least 0.1% by weight, at least 0.2% by weight, at least 0.3% by weight, at least 0.4% by weight, at least 0.5% by weight, at least 0.6 by weight, at least 0.7% by weight, at least 0.8% by weight, at least 0.9% by weight, or at least 1% by weight.

An organic nitrogen source can comprise amino acids or nucleotides, or compounds comprising them, such as peptides, oligopeptides, polypeptides, oligonucleotides and nucleic acids. In addition, an organic nitrogen source can comprise any one or more of: a yeast extract, a peptone, and amino acid mixture, a hydrolyzed corn protein, a hydrolyzed soy protein, a hydrolyzed pea protein, and a corn steep liquor. Certain agricultural byproducts containing high amounts of nitrogen can also be used as organic nitrogen sources.

An inorganic nitrogen source can comprise any one or more of: a nitrate salt, a nitrite salt, an ammonium salt, a urea compound, nitrogen gas, and ammonium hydroxide.

### c) Fungal Nutrients

Additional fungal nutrients can comprise any one or more of: salts of magnesium (e.g., magnesium sulfate heptahydrate (MgSO₄·7H₂O)), salts of calcium (e.g., calcium sulfate (CaSO₄)), salts of copper (e.g., copper sulfate (CuSO₄)), salts of manganese (e.g., manganese sulfate (MnSO₄)), ammonium salts (e.g., ammonium sulfate ((NH₄)₂SO₄)), salts of zinc (e.g., zinc sulfate (ZnSO₄)), iron-ammonium salts (e.g., ferric ammonium sulfate (FeNH₄(SO₄)₂) and ferrous ammonium sulfate (Fe(NH₄)₂(SO₄)₂)), potassium salts (e.g., potassium hydrogen phosphate (i.e., monopotassium phosphate KH₂PO₄ or dipotassium phosphate K₂HPO₄)), sodium phosphates (i.e., Na₃PO₄, Na₂HPO₄, NaH₂PO₄), ammonium nitrate (NH₄NO₃), biotin, acetic acid and sodium acetate. Other salts of calcium, magnesium, iron, zinc, copper, manganese and molybdenum can also be used as nutrients.

### d) Exemplary Fungal Culture Media

In certain embodiments, the second culture medium is Czapek-Dox medium. In additional embodiments, the second culture medium is Czapek-Dox medium and the fungus is Aspergillus oryzae. In additional embodiments, the second culture medium is Vogel's medium. In further embodiments, the second culture is Vogel's medium and the fungus is Fusarium 10 venenatum. In yet additional embodiments, the second culture is Vogel's medium and the fungus is Neurospora intermedia.

Accordingly, in certain embodiments, the culture medium is Czapek-Dox medium and contains:
30 g/L sucrose
2 g/I NaNO₃
1 g/L K₂HPO₄
0.5 g/L MgSO₄
0.5 g/L KCl
0.01 g/L FeSO₄.

In additional embodiments, the culture medium is Vogel's medium and contains:
15 g/L sucrose
3 g/I trisodium citrate
5 g/L K₂HPO₄
2 g/L NH₄NO₃
0.2 g/L MgSO₄
0.1 g/L CaCl₂
0.25 mg/ml biotin
5 mg/L citric acid
5 mg/L ZnSO₄
1 mg/L Fe(NH₄)₂(SO₄)₂
0.25 mg/L CuSO₄
0.05 mg/L MnSO₄.

In additional embodiments, a fungal culture medium contains:
3 g/L malt extract
10 g/L dextrose
3 g/L yeast extract
5 g/L peptone.

### 2. Fungal Culture Conditions

To manufacture a composite material for use as a food product, a second culture medium containing a cellulose-containing scaffold manufactured as described above, can be inoculated with a fungus to initiate the second culture. In certain embodiments, the scaffold is submerged or immersed in the second culture medium. In certain embodiments, the inoculum is a logarithmic-phase culture of fungus comprising 1-10% wet weight of the culture. In other embodiments, the fungal inoculum comprises 1-10% of the volume of the second culture medium. In additional embodiments, the fungal inoculum comprises 1% or more, 2% or more, 3% or more, 4% or more, 5% or more, 6% or more, 7% or more, 8% or more, 9% or more, or 10% of wet weight of the culture.

a) pH

The pH of the medium can be adjusted if desired using, for example, phosphate, carbonate, tris, acetate, citrate, HCl, acetic acid, imidazole, bicarbonate or NaOH. The pH can initially be adjusted in the culture to an acidic pH, e.g., between about 4 and 5.5, e.g., about 4.5 to about 4.8, e.g., about pH 5.2 or about pH 4.7. Growing bacteria will acidify the culture and reach pH of between about pH 2 to about pH 5. In certain embodiments, the culture, after initial growth, is maintained, e.g., through buffering, at between about pH 2 to about pH 6, e.g., between pH 2 to about pH 5.

### b) Temperature

Fungal cultures are conducted at a temperature suitable for production of the desired composite material. Culture can be conducted at a temperature between 4° C. and 50° C., or between 20° C. and 40° C., or between 20° C. and 30° C., or between 25° C. and 35° C., or between 27° C. and 32° C., or between 27° C. and 28° C., or at 27° C., or at 27.5° C.

### c) Humidity

The humidity of the environment in which the culture is contained contributes to the moistness of the composite material. Accordingly, the relative humidity (RH) of the second culture can be controlled to optimize production of a suitable food product. For example, fungal culture can be conducted at a relative humidity of between 10% and 90%, or between 20% and 80%, or between 30% and 70%, or between 40% and 60%, or between 45% and 55%. In certain embodiments, the fungal culture is conducted at a RH of 55-65% or at a RH of 60%.

### d) Oxygenation

The oxygen concentration of the fungal culture can also be controlled, e.g., by sparging (e.g., bubbling) air into the culture and/or by adjusting the surface area of the culture. The dissolved oxygen concentration of the second culture medium can be between 2% and 20% by volume. For example, the dissolved oxygen content of the second culture medium can be about any of 2%, 3%, 4%, 5%, 6%, 7%, 8%, 9%, 10%, 11%, 12%, 13%, 14%, or 15%. Optimal oxygen concentration depends on the species of fungus being cultured. Increased aeration (i.e., higher dissolved oxygen) can contribute to a composite material with a lower cutting force.

### e) Length of Culture

The duration of the second culture can be selected to provide a composite material with desired properties, such as flavor, thickness and texture. Accordingly, the second culture can be conducted for 1 to 20 days or more. For example, fungal culture can be conducted for 7 days, for 8 days, for 9 days, for 10 days, for 11 days, for 12 days, for 13 days, for 14 days, for 15 days, for 16 days, for 17 days, for 18 days, for 19 days or for 20 days. In certain embodiments, fungal culture is conducted for 2-20 days, for 4-16 days, for 10-14 days, for 4-30 days, 4-18 days for 5-14 days, for 7-12 days, or for 8-10 days. For extended cultures (e.g., 2 weeks or more), the second culture medium can be supplemented with one or more of the carbon source, the nitrogen source or the nutrient; or the culture medium can be replaced with fresh culture medium, either the same as, or different from, the original culture medium; or the culture can be re-inoculated with either the same, or different, fungus.

Cultures can be conducted in static fashion or can be agitated.

Static cultures of A. oryzae can be conducted in Czapek-Dox medium (supra) adjusted to pH 6.8, at 27° C., using an inoculum of 10% (v/v). Static cultures of Neurospora spp. are conducted in Vogel's medium (supra) adjusted to pH 6.0, at 30° C., using an inoculum of 10% (v/v). Static cultures of Fusarium venenatum are conducted in Vogel's medium (supra) adjusted to pH 6.0, at 30° C., using an inoculum of 10% (v/v).

Agitated cultures of A. oryzae can be conducted in Czapek-Dox medium (supra) adjusted to pH 6.8, at 27° C., using an inoculum of 10% (v/v); with a dilution rate of 0.2/hour, an aeration rate of 0.25 vvm and a stir rate of 180 rpm. Agitated cultures of Neurospora spp. are conducted in Vogel's medium (supra) adjusted to pH 6.0, at 30° C. using an inoculum of 10% (v/v); with a dilution rate of 0.2/hour, an aeration rate of 0.25 vvm and a stir rate of 120 rpm. Agitated cultures of Fusarium venenatum are conducted in Vogel's medium (supra) adjusted to pH 6.0, at 30° C. using an inoculum of 10% (v/v); with a dilution rate of 0.2/hour, an aeration rate of 0.25 vvm and a stir rate of 120 rpm.

Additional information regarding culture conditions, culture vessels and culture systems is provided in International Publication WO 2022/245683; the disclosure of which is incorporated by reference, in its entirety, for the purpose of providing information about methods, compositions and systems for the culture of bacteria and fungi.

### B. Animal Cell Culture

Various media for use in the culture of eukaryotic cells are known in the art. See, generally, Freshney, R. I., “Culture of Animal Cells: A Manual of Basic Technique,” Fifth Edition, Wiley, New York, 2005. Media such as minimal essential medium (e.g., α-MEM) and Eagle's medium can be used and supplemented with, for example, serum (e.g., fetal bovine serum) at 5-20% or any value therebetween), glutamine, penicillin and/or streptomycin. Serum-free media are also known in the art.

Animal cells are cultured in the polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel) that is in a medium described above. They are then placed in a bioreactor with the scaffold or produced in a solid-state and cultured at the following standard environmental conditions 5% CO₂, 37° C., 95% humidity. The cells are allowed to culture until formed spheroids adhere to, and continue to proliferate on, the scaffold. Spheroids can take weeks to form and continue to expand throughout the three-dimensional scaffold. When the mass of spheroids has reached at least 5% w/w, the composite material of animal cells and a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel) is harvested. To halt the proliferation of the cells, the composite material is frozen at −20° C. for at least 40 minutes.

### C. Plant Cell Culture

Plant cell types are cultured in Murashige and Skoog (MS Media) and Gamborg's B5 Medium using seed, meristem, callus, or bud culture. The plant cells are cultured under a photosynthetic photon flux density (PPFD) of 26 μmol m-2 delivered by cool white fluorescent tubes with a photoperiod of 16/8 h (day/night) at 25±2° C. After at least one week, the proliferation of cells is halted by freezing at −20° C. for at least 40 minutes.

### V. Co-Culture of Bacteria and Fungi

In certain embodiments the composite material is produced by coculture of one or more bacteria in one or more fungi. Processes for producing a culture of bacteria and fungi are described in, e.g., International Publication WO 2022/245683. In brief, (a) combining, in a fermentation vessel, a starter culture and a culture medium, to produce a culture, wherein: (i) the starter culture comprises one or more fungi and one or more cellulose-producing bacteria; and, (ii) the culture medium comprises water, a carbon source, a nitrogen source, and nutrients. Culture conditions can include those as described herein for the culture of bacteria and for fungi.

In an exemplary embodiment of the culture medium can comprise a combination of:

### Mixture 1

35 g glucose
25 g fructose
500 mL of 100 mM acetate buffer in distilled water, pH=4.6

Components are stirred to dissolve and autoclaved at 121° C. for 20 minutes.

### Mixture 2

2.5-7.5 g yeast extract, peptone, amino acids
2 mL v/v ethanol (optional)
5 g KH₂PO₄,
2 g NH₄NO₃
0.2 g MgSO₄ 1 g CaSO₄
0.005 g Zn SO₄
0.001 g Fe(NH₄)₂(SO₄)2 0.00025 g CuSO₄ 0.0001 g MnSO₄
0.0025 g biotin
500 mL of 100 mM acetate buffer in distilled water, pH=4.6

Mixture 1 and mixture 2 are then combined to produce 1 L of culture medium.

The culture medium is inoculated with bacteria and fungi. The culture is allowed to grow for about 14 days to produce a pellicle of composite material comprising a scaffold of bacterial cellulose and fungal protein.

Pellicle can be processed as described herein. For example, the pellicle can be boiled in and alkaline or acidic solution to kill bacteria and fungi. The product can be cut into properly sized pieces and marinated in a solution to provide desired flavor and color.

### VI. Isolation and Properties of Composite Materials

### A. Isolation

Following the second culture, the composite material is harvested from the culture medium. The culture medium can be drained off the composite material or the composite material, being a solid, can be manually removed from the culture vessel.

### B. Properties

Composite materials of this disclosure can comprise, for example, by wet weight, about 60%-about 80% (e.g., about 70%) water, about 10% to about 14% scaffold (e.g., bacterial cellulose), and about 10% to about 16% protein (e.g., protein from eukaryotic cells populating the scaffold). They can comprise, by dry weight, about 33% to about 47% scaffold, (e.g., bacterial cellulose), and about 33% to 53% protein (e.g., protein from eukaryotic cells populating the scaffold). Structurally, the product can comprise live eukaryotic cells, dead eukaryotic cells or cellular material from the eukaryotic cells. Cellular material can include the cell (living or dead) in which the material resides and/or the cell (living or dead) that produced the material. Cellular material further refers to the remains of cells that exist outside of a living or dead cell. Typically, cellular material comprises a mixture of biomolecules produced by a cell (e.g., nucleotides, nucleic acid, amino acids, polypeptides, protein, sugars, polysaccharides, starches, and lipids such as fatty acids and triglycerides). For example, the material can comprise single cell protein (e.g., dead fungal cells) embedded in a scaffold of, e.g., bacterial cellulose. In certain embodiments, cellular material comprises mycoprotein. “Cell residue” refers to any cell-free cellular material.

Composite materials of this disclosure can be free or essentially free of bacterial cellular material. Certain composite materials can be free or essentially free of DNA encoding bacterial 16S rRNA. In some embodiments, no more than any of 5%, 4%, 3%, 2%, 1%, 0.5%, or 0.1% of DNA in the material the composite material encodes bacterial 16S rRNA.

### 1. Protein Content

The composite material of this disclosure can have a protein content from the eukaryotic cells of at least 5% protein by dry weight. For example, the composite material can have a protein content of at least any of 6%, 7%, 8%, 9%, 10%, 11%, 12%, 13%, 14%, 15%, 16%, 17%, 18%, 19% or 20%, 25%, 30%, 35%, 40%, 45% or 50% protein by dry weight, e.g., protein sourced from eukaryotic cells that populated the scaffold. The composite material also can have protein content of less than 5% dry weight.

In another embodiment, the composite material comprises no more than 5% fungal protein by dry weight.

In some embodiments, the protein in the composite material comprises single-cell protein. The single-cell protein can be derived substantially or solely from fungal protein, e.g., it can be fungal protein or mycoprotein. The protein can be a whole protein; i.e., having a protein digestibility amino acid score (PDCAAS) of any of 0.8, 0.85, 0.9, 0.95, or 1.0; for example, a PDCAAS equal to at least 0.9 (e.g., 1.0).

### 2. Fiber Content

The composite material of this disclosure has a fiber content of at least 5% fiber (e.g., cellulose) by dry weight. For example, the composite material can have fiber (e.g., cellulose) content of at least any of 5%, 10%, 14%, 15%, 20%, 25%, 30%, 35%, 40%, 45%, 50%, 55%, or 60% fiber by dry weight. In some embodiments, fiber content is between about 5% and about 60%, between about 10% and about 50%, between about 15% and about 40%, between about 20% and about 40%, or between about 30% and about 40%, by weight. The fiber content of food products can be determined using the AOAC method 991.43. (See, e.g., acnfp.food.gov.uk/sites/default/files/mnt/drupal_data/sources/files/multimedia/pdfs/annexg.pdf.) In certain embodiments, the fiber comprises bacterial cellulose.

### 3. High Nutrient Content of Composite Materials

Accordingly, certain products of this disclosure satisfy the FDA definitions of high protein and high fiber. Pursuant to FDA 21 C.F.R. § 101.54, a product has a high nutrient content if the content is 20% or greater of the Recommended Daily Intake (“RDI”) per reference amount customarily consumed (RACC).

The RDI for protein is 50 g for adults and children aged 4 or older. In some embodiments, the composite materials disclosed herein contain 10-15 grams protein per RACC (85 g) for foods such as fish and shellfish, which is about 20-50% of the RDI, thereby qualifying as “high protein.” In some embodiments, the composite material has at least any of 10 g, 11 g, 12 g, 13 g, 14 g, 15 g, 16 g, 17 g, 18 g, 19 g or 20 g of protein per RACC for foods such as fins and shellfish.

The RDI for dietary fiber is 28 g. In some embodiments, the composite materials disclosed herein contain as much as 10-14 grams dietary fiber per RACC (85 g) for foods such as fish and shellfish, which is about 35-50% of the RDI, greater than the amount needed to qualify as “high fiber.” In some embodiments, the composite material has at least any of 10 g, 11 g, 12 g, 13 g, 14 g, 15 g, 16 g, 17 g, 18 g, 19 g or 20 g of fiber per RACC for foods such as fin fish and shellfish.

### 4. Other Properties of Composite Materials

The composite material can have a cutting force in the same ranges as a pellicle from which it is formed. In some embodiments, further processing of the pellicle, for example, by boiling, puncturing, and other tenderizing methods, can produce a composite material with a cutting force less than a freshly harvested pellicle.

Crystallinity (or the crystallinity index) is a property of cellulose that describes the relative abundance of ordered cellulose fibers, as compared to amorphous cellulose. It is expressed as the ratio of crystalline cellulose to amorphous cellulose as determined by, for example, X-ray crystallography, nuclear magnetic resonance (e.g., ¹³C NMR) and Fourier transform infrared spectroscopy. In certain embodiments, the crystallinity of the composite material is 30-90% or 40-80% or 50-70% or 55-65%. In additional embodiments, the crystallinity of the composite material is at least 10%, at least 20%, at least 30%, at least 40% or at least 50%.

Tensile strength is the maximum tension that a material can withstand, while being stretched or pulled, before breaking. The tensile strength is a measure of its elasticity, equal to the ratio of the stress acting on a substance to the strain produced. In certain embodiments, the tensile strength of the scaffold is 0.05 to 5 kg, or 0.06 to 4 kg, or 0.07 to 3 kg, 0.08 to 2.5 kg, 0.09 to 2.25 kg or 0.1 to 2 kg. In certain embodiments, the tensile strength of the composite material is between 150 grams and 2,000 grams. In other embodiments, the tensile strength of the composite material is between 0.1 and 20 mPa (milliPascals), or between 0.2 and 18 mPA, or between 0.3 and 16 mPa, or between 0.4 and 14 mPa, or between 0.5 and 13 mPa, or between 0.75 and 12 mPA or between 1 and 11 mPa or between 2 and 10 mPa, or between 3 and 9 mPa, or between 5 and 7 mPa. In additional embodiments, the tensile strength of the composite material is between 2 and 10 mPa.

The storage modulus of a material is the ability for a material to resist deformation; and is representative of its elastic or solid-like properties. At 0.1% strain the storage modulus range between the composite material herein and tuna have storage moduli between 110 kPa and 20 kPa. The composite material product begins to break down at a low strain rate of 0.398% compared to seafood which requires higher amounts of deformation at 1% strain for breakdown to be observed. At 1% strain, the storage modulus range between the composite material product and tuna are 91 kPa and 27 kPa. At 100% strain, where there is mastication of the material, the composite material product exhibits a modulus under 10 kPa, similar to tuna and calamari.

The loss modulus of a material is the ability for a material to dissipate energy and is representative of its viscous or liquid-like properties. In general, the loss modulus is much lower than the storage modulus at all strain rates across their corresponding group, indicating that both seafood and the composite material product behave mainly as a solid, rather than a liquid.

The loss modulus range at 0.1% for the composite material product and tuna are between 43 kPa and 6 kPa. The range at 1% is between 36 kPa and 6 kPa. At 100% strain, the loss modulus of the composite material product and seafood are all below 6 kPa.

### VII. Processing the Composite Material

Isolated and/or purified composite materials can be processed in a number of ways to modify the texture, shape, flavor, color, appearance and/or nutritional content of the composite material.

### A. pH Adjustment

The pH of the harvested composite material can be adjusted, using buffers that are known in the art. For example, to neutralize an acidic product, sodium hydroxide or imidazole buffers can be used. pH adjustment is accomplished by washing the material with the buffer and monitoring the pH of the runoff, until the desired pH is reached. Alternatively, the material can be soaked in a buffer and monitored until the desired pH is achieved. In certain embodiments, the pH of the harvested composite material is adjusted to between pH 4 and pH 7, depending on the desired flavor of the product.

### B. Killing Eukaryotic Cells

If a composite material devoid of eukaryotic, e.g., fungal, cells (e.g., containing fungal protein and bacterial scaffold) is desired, living eukaryotic cells, e.g., fungi, remaining in the composite material can be killed. Eukaryotic, e.g., fungal, cells can be killed by boiling the composite material (e.g., in water or an aqueous buffer such as sodium acetate), by pasteurization, and/or by irradiation (e.g., exposure to ultraviolet light having a wavelength of 240-300 nm). In one embodiment, composite material containing fungal biomass grown in a scaffold of, e.g., bacterial cellulose is held at elevated temperature (e.g., 70-80° C.) for 20 minutes. If not immediately consumed or processed, the material can then be stored in food-grade packaging, with or without water, at 4° C. Accordingly, the product can be essentially free of living eukaryotic cells. Eukaryotic cells can be killed by methods described herein. For example, the material can be boiled in an acidic solution.

### C. Marinating

Composite material that has been isolated and/or purified can be marinated to modify its flavor and/or texture. Marinade solutions can comprise, for example, water, oils, vinegars, spices, salt, and pepper. In addition to modifying flavor and texture, marinating can be used to add nutrients and stabilizers to the composite material. Additional components of marinades include yeast extract, omega-3-algal oil, canthaxanthin, nutrients (e.g., niacin, reduced iron, thiamine mononitrate, riboflavin, folic acid cobalamin and mixtures thereof), natural and/or artificial flavors, guar gum, trehalose and ions (such as, for example, calcium, magnesium, manganese, sodium, potassium, ferrous).

A fish flavor can be imparted to the product by including, as a flavoring agent, salt, trimethyl amine (TMA), sugar and/or algal oils.

Natural flavors include, but are not limited to, essential oils, oleoresins, essences or extractives, protein hydrolysates, distillates, or any product of roasting, heating or enzymolysis, which contains the flavoring constituents derived from a spice, fruit or fruit juice, vegetable or vegetable juice, edible yeast, herb, bark, bud, root, leaf or similar plant material, meat, seafood, poultry, eggs, dairy products, or fermentation products thereof, whose significant function in food is flavoring rather than nutritional. Natural flavors include the natural essence or extractives obtained from plants.

Artificial flavors include, but are not limited to, any substance, the function of which is to impart flavor, which is not derived from a spice, fruit or fruit juice, vegetable or vegetable juice, edible yeast, herb, bark, bud, root, leaf or similar plant material, meat, fish, poultry, eggs, dairy products, or fermentation products thereof.

Exemplary flavoring agents also include extracts, oleoresins or essential oils of seeds, herbs and spices; as well as yeast extracts, and Flavor Extract Manufacturer's Association (FEMA) materials No. 1-4980.

Additional flavoring agents include volatile organic compounds, amino acids, nucleotides (e.g., adenosine, guanosine and inosine monophosphates) and organic acids (e.g., tartaric, malic, lactic, acetic, citric, tannic and succinic acids).

Oils used in marinades can include any type of vegetable or animal oil; e.g., olive oil, avocado oil, rapeseed (canola) oil, sesame oil, soybean oil, sunflower oil and various types of fish oil. Algal oils containing omega-3-fatty acids can also be used. For example, there are various types of “omega-3-algal oils” in which a vegetable oil is loaded with 10%-90% of an algal oil containing one or more of the omega-3-fatty acids docosahexaenoic acid (DHA), eicosapentaenoic acid (EPA) or alpha-linoleic acid (ALA).

Nutrients used in marinades include, for example, dicalcium phosphate, niacinamide, calcium-D-pantothenate, pyridoxine hydrochloride, riboflavin, potassium iodide, sodium selenite, cyanocobalamin, and ferrous sulfate.

One or more stabilizing agents can be included in a marinade to enhance water retention in the product. In general, such stabilizing agents include sugars, sugar alcohols, starches and gums. Exemplary stabilizing agents include guar gum and related substances, such as gum arabic, xanthan gum, starches (modified and unmodified), dextrins, maltodextrins and fibers such as, for example, cellulose, psyllium or inulin. Starches can be modified by, for example, thermal modification, hydrolysis (e.g., acid-catalyzed hydrolysis, alkaline hydrolysis, enzymatic hydrolysis, dextrinization), etherification (e.g., methylation, hydroxymethylation, hydroxypropylation, carboxymethylation), esterification (e.g., acetylation, fatty acylation, phosphorylation, succinylation), cationization, cross-linking (e.g., using dicarboxylic acids, or alkaline crosslinking) or a combination of one or more of the foregoing techniques.

Additional stabilizing agents include trehalose and related substances, such as monosaccharides, disaccharides, oligosaccharides, polyols and sugar alcohols.

The aforementioned stabilizing agents, among other properties, limit the drip loss of the material after freezing and thawing; drip loss being the amount of mass lost to water released after thawing. In certain embodiments, the presence of a stabilizing agent limits drip loss to 5-40%; i.e., no more than about any of 5%, 10%, 15%, 29%, 25%, 39%, 35% or 40% of the mass of the material prior to freezing.

Marinade solutions can also contain a source of calcium ions such as, for example, calcium sulfate, calcium chloride, calcium gluconate, calcium lactate or calcium gluconate lactate. Calcium ions aid in crosslinking; for instance, the crosslinking of sodium alginate in a HIPE (see below).

In one embodiment, a marinade solution is an aqueous solution containing one or more of the following ingredients:
0.0-5.0% (w/v) yeast extract
0.0-1.5% (w/v) salt (e.g., NaCl)
0.0-0.5% (w/v) omega-3-algal oil
0.05-0.5% (w/v) canthaxanthin
0.0-0.5% (w/v) nutrient mixture
0.0-0.5% (w/v) flavoring agent
0.0-0.5% (w/v) guar gum
0-20% t (w/v) trehalose
0.0-1.5% (w/v) calcium ion source

A marinade solution containing all of the components set forth above is useful for imparting a flavor of tuna or salmon to a composite material. A fish flavor can be imparted by marinating in sugar, trimethyl acetate and salt. Flavor can be enhanced by the addition of amino acids and, in the case of red fishes, iron.

Marinating is performed by, for example, rotating a mixture of composite material and marinade solution under vacuum. The ratio (weight/volume) of composite material to marinade solution can range from 1:5 to 5:1. Thus, in certain embodiments, the weight/volume ratio of composite material to marinade solution is 1:5, 2:5, 3:5, 4:5, 1:1, 2:1, 3:1, 4:1 or 5:1. Rotation can be conducted, for example, at 5-20 rpm for 20 minutes. The “pick-up” value; i.e., the amount of marinade absorbed by the composite material, can be up to 40% of the weight of the composite material; i.e., 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%. Once marination is complete, the marinated composite material can be used as is, or further processed by, for example, cutting, shaping, tenderizing, layering and/or cooking.

### D. Cutting and Shaping

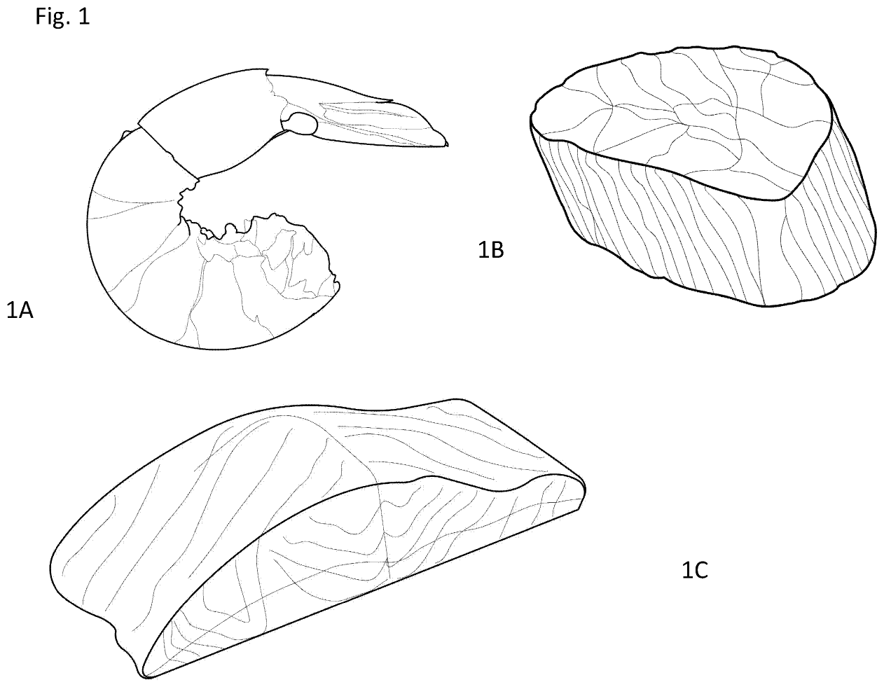

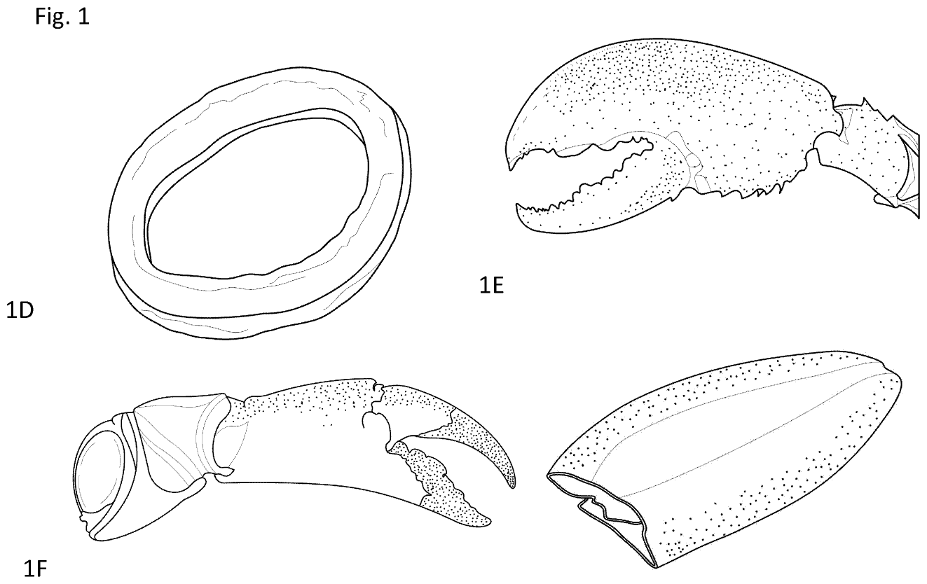

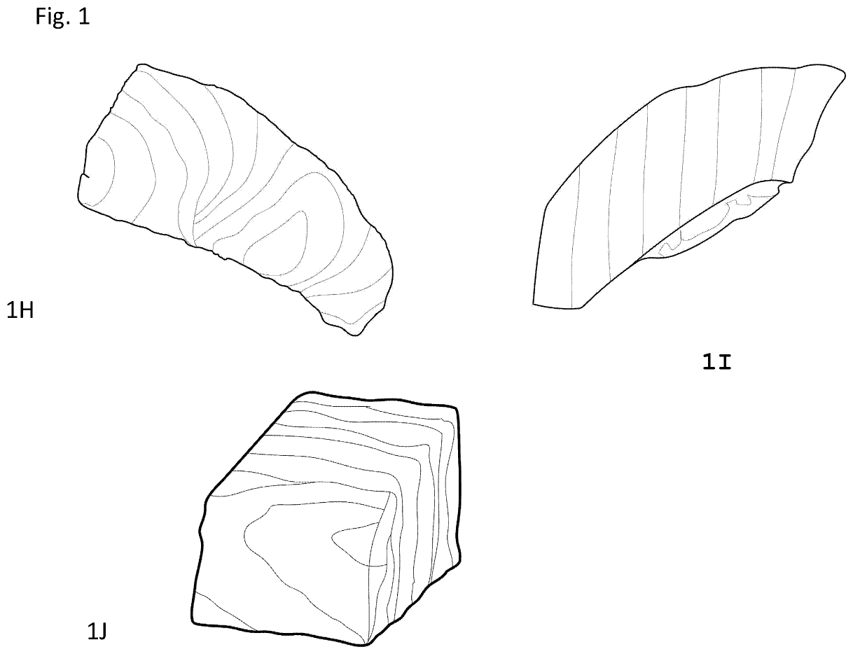

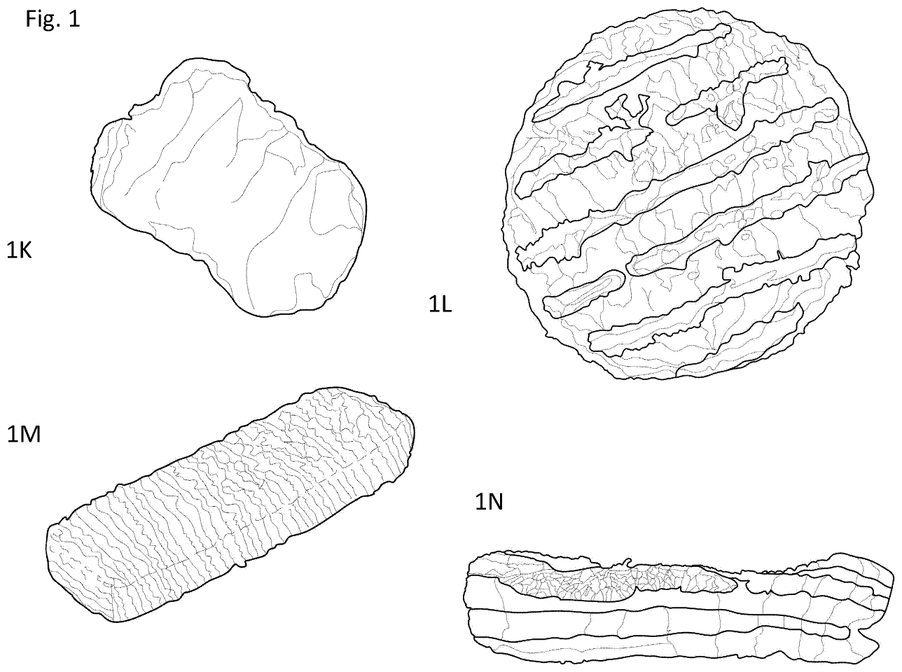

Composite material in the form of a slab of appropriate width, length and thickness can be formed into shapes that are attractive as food products. In the production of sushi products, chefs will use a block of fish. Such blocks can have dimensions of about 2.5 cm×2.5 cm×5 cm. Typically, a piece (e.g., a slab) of the material can be cut into a plurality of pieces having desired shapes. Shapes include geometric or fanciful shapes, such as closed curvilinear shapes (e.g., circles, ovals, and ellipses), and polygons (e.g., rectangles, squares, hexagons, and stars). Shapes include, for example, discs, logs, strips, medallions, crescents, fans, rectangles, triangles, rings, slabs, etc. Materials can also be shaped as food products, for example, seafood products or poultry (e.g., chicken) products. Seafood product shapes include, for example, sushi, sashimi, shrimp, crab, lobster, squid/calamari. Shapes can include textures associated with the particular food product. Materials also can be shaped, for example as patties, chicken nuggets, bacon strips, burgers, or sausages. Exemplary shapes for composite materials are shown in FIGS. 1A-1N. These include: shrimp (1A), scallop (1B), salmon steak (1C), calamari ring (1D), lobster claw (1E), crab claw (1F), calamari mantle (1G), sushi shapes (1H, 1I, 1J), nugget (1K), patty (1L), stick (e.g., fish stick) (1M), and bacon (1N).

Forming into shapes can be accomplished by cutting or sculpting a piece of the composite material. Cutting can be done with a cutting implement, for example, a knife, a wire, a cookie cutter, or a die. Composite material can be diced into pieces with a die. In another embodiment, the composite material can be shaped by a mold during its formation. For example, a cover having shapes cut out of it can be placed on the surface of either the first culture medium or the second culture medium. The scaffold (in the first culture medium) or the composite material (in the second culture medium) will grow where the surface is exposed to oxygen, i.e., through the holes formed by the cut-out shapes, thereby taking the shape of the hole. So, for example, a mold can comprise a plurality the same or different shape(s). Accordingly, a shaped product can be a product produced by a process of cutting or of molding.

In additional embodiments, the shape and/or texture of a composite material can be modified by extrusion.

### E. Tenderizing

Harvested composite material can be tenderized, if desired. Tenderization can be achieved manually, e.g., using needles; thermally, e.g., by mild par-cooking; and/or enzymatically, e.g., by treating the composite material with a cellulase (e.g., Celluclast*) and/or a protease (e.g., pepsin, trypsin, chymotrypsin, pronase, papain, bromelain, etc.). Boiling in an acidic solution also contributes to tenderizing.

### F. Layering

In some embodiments, a composite material, either marinated or unmarinated, is formed into layers with a second material to, for example, simulate the appearance and/or texture of a food such as salmon. Exemplary second materials include various types of emulsion.

In certain embodiments the second material is a high internal phase emulsion (HIPE). A HIPE is an emulsion in which the internal phase volume percent exceeds 74%, and is typically solid at room temperature. In one embodiment, a HIPE is formed by combining a water phase and an oil phase. The water phase can contain a hydrocolloid (e.g., a carrageenan such as kappa carrageenan or iota carrageenan, or agar agar) and one or more crosslinking agents (e.g., an enzyme such as a transglutaminase or a laccase, or an alginate such as sodium alginate). The oil phase can contain an algal oil such as omega-3-algal oil, a seed oil, a food-grade wax (e.g., candelilla wax, carnauba wax, beeswax, rice bran), and/or an emulsifier such as, for example, glycerol monostearate.

To formulate a HIPE, an aqueous phase and an oil phase are separately heated (e.g., to approximately 70° C. or higher, depending on the components of the phases). The heated aqueous phase is subjected to shear agitation at a speed of at least 1200 rpm, while the heated oil phase is slowly added. After addition of the oil phase, agitation is continued as the emulsion cools. When the emulsion cools to an appropriate temperature (e.g., 50° C., 45° C., 40° C., 35° C., 30° C., 25° C., or 20° C.) it is used for layering. The emulsion can contain between 50-90% aqueous phase and between 10-50% oil phase, depending on the desired properties of the layered product.

In certain embodiments, the aqueous phase contains:
0.1-1.0% kappa carrageenan
0.1-1.0% iota carrageenan
0.05-5.0% sodium alginate
0.0-5.0% crosslinking agent

In additional embodiments, the oil phase contains:
98.0-99.8% omega-3-algal oil
0.1-1.0% candelilla wax
0.1-1.0% glycerol monostearate

To make a layered product, a first sheet of composite material (i.e., fungal biomass contained in a scaffold of, e.g., bacterial cellulose), having a thickness between 1-50 mm, is laid flat and a layer of HIPE (or other second material), between 0.05 and 5.0 mm thick is spread across the sheet of composite material using, e.g., a roller or a spreader nozzle or an enrober. A second sheet of composite material is then laid on the coated first sheet. Additional steps of spreading the second material onto uncoated composite material and addition of a sheet of composite material on top of the spread second material can be continued as necessary, until a product having the desired thickness and/or number of layers is obtained. For storage, the final product can be blast frozen and stored (e.g., in a polyethylene bag) at reduced temperature (e.g., 4° C., 0° C., −20° C., −70° C.) in an inert atmosphere (e.g., under nitrogen or argon) and/or under vacuum.

### G. Grinding

Composite material can be ground, and the ground material used in the preparation of a number of food products. For example, composite material that is ground to an average particle diameter of about 6 mm (optionally including one or more of spices, protein, starch, color, flavorings or nutrients) is used as a ground meat substitute (e.g., for pasta sauce). The ground composite material can also be shaped into burgers or patties to simulate hamburgers or sausage patties. In further embodiments; ground composite material, with or without one or more of the optional ingredients mentioned above, is stuffed into casings, and the stuffed casings are par-cooked (i.e., partially recooked for subsequent re-heating) to simulate sausage (e.g., Italian sausage, bratwurst, polish sausage, Chinese sausage). A similar procedure using a smaller grind diameter (e.g., about 1-2 mm) can be used to produce simulated frankfurters.

In additional embodiments, composite material, ground to an average particle diameter of about 6 mm or less, and optionally including one or more of spices, protein, starch, color, flavorings, fat, vegetable or nutrients, can be encased in dough and cooked to simulate, e.g., dumplings, empanadas, shumai, gyoza, pierogi or ravioli.

In additional embodiments, composite material (optionally including one or more of spices, protein, starch, color, flavorings or nutrients) is ground to an average particle diameter of about 3-4 mm and dehydrated (e.g., to <0.8 water activity). The dehydrated material is used for soup, broth, stock, or pasta (e.g., stuffed into ravioli or manicotti, or in pre-made lasagna.

In additional embodiments, ground composite material can be used to make a dumpling. For example, the ground material can be stuffed into a dope pocket. In certain embodiments, the dumpling comprises composite material having shrimp flavor.

Composite material can also be used to manufacture protein powders by dehydrating the composite material and grinding it to an average particle diameter of less than about 150 microns. The ground material can optionally be mixed with flavoring(s) and additional nutrient(s) and packaged, e.g., for use as a nutritional supplement, for addition to milkshakes and other liquid beverages, or as a ready-to-drink product.

In additional embodiments, composite materials can be used to produce dairy products. For liquid dairy products such as milk, cream, yogurt and kefir, protein powder made as described above is emulsified with one or more of water, fat, sugar, additional protein; texturizing agent(s) such as, for example, carrageenans and/or hydrocolloids; flavoring or nutrients.

For solid dairy products such as cheese, ice cream, cream cheese and sour cream; protein powder made as described above is emulsified with one or more of water, fat, sugar, additional protein; texturizing agent(s) such as, for example, hydrocolloids, starches and/or gums; flavoring or nutrients.

The consistency of a product such as a dairy product (i.e., whether it is a liquid or a solid) is determined by the relative amounts of water, fat and/or hydrocolloid that are present in the product. For example, higher relative amounts of water (or aqueous phase) generate products that are more liquid; while higher amounts of fat and/or hydrocolloid generate products that are more solid.

In additional embodiments, composite materials can be used as animal feed by emulsifying a composite material as described herein with one or more of water, fat, sugar, additional protein, texturizing agent(s) such as hydrocolloids and/or starches, flavoring or nutrients. The emulsified material is diced or extruded, and packaged. For example, simulated fish is extruded to make cat food; and simulated beef or pork is extruded to make dog food. The emulsified material can be dried and flaked to make fish food. Such material can also be used to supplement feed for farm animals.

### H. Coloring

Various dyes and colorings (e.g., xanthan, lycopene, beet color, dyes) can be added as coloring agents or colorants to composite materials to make the appearance of the material similar to that of a food product of interest. Non-toxic, edible dyes are known in the art.

Any non-toxic natural or artificial color can be used as a coloring agent in a marinade. Extracts and oleoresins of natural products can also be used to modify the color and/or appearance of the material. In one embodiment, canthaxanthin is used as a coloring/appearance agent.

### I. Flavoring

Various flavorings (e.g., MSG, yeast extract, Marmite®, spices, herbs, barbecue sauce, teriyaki sauce, soy sauce, anchovy paste, salt, Worcestershire sauce, curry) can be added as flavoring agents or flavorants to alter the taste of a composite material, as described elsewhere herein.

Flavors (as well as nutrients) can be provided by a variety of yeast extract products. Yeast extracts are products, typically dissoluble in water and fats, produced from baking yeast by autolysis or enzymic hydrolysis. In some embodiments, the yeast extracts comprise comprises free 5′ ribonucleotides. These can be produced by, for example, subjecting yeast to autolysis to produce RNA, and converting the RNA to 5′ ribonucleotides.

Yeast extracts for use as flavorants are commercially available. These include, for example, the Maxavor™ line of products from DSM™ (Delft, The Netherlands), and Savorboost™ from Ajinomoto™ (Ontario, California).

### J. Nutrients

Various nutrients (e.g., vitamins, proteins, electrolytes, minerals) can be added as supplemental nutrients to alter the nutritional properties of a composite material, as described elsewhere herein. Exemplary nutrients include niacin, reduced iron, thiamine (e.g., thiamine mononitrate), riboflavin, folic acid and cobalamin. Nutrients further include yeast extracts, and fats, such as omega-3 fatty acids.

Nutrients can include one or more of the following: Potassium chloride, zinc oxide, ferric sulfate, niacin amide, cyancobalamin, thiamine mononitrate, pyridoxine hydrochloride, pantothenic acid, sodium selenite, potassium iodide, and niacinamide.

### K. Emulsifying

Composite materials can be formed into emulsions, using various emulsifying agents as described elsewhere herein, to modify the texture and mouthfeel of a food product made from a composite material.

### L. Cooking

Layered or unlayered composite material can be consumed raw of after cooking. For consumption raw, the composite material can be cut to size for sushi, sashimi, nigiri, chirashi, maki, uramaki, temaki, poke or other preparations that use raw fish or seafood.

Composite material can be cooked using either wet heat or dry heat. Wet heat includes procedures in which heat is transferred to the composite material by liquid or steam. Examples of wet heat include poaching, boiling, blanching, braising, steaming, simmering, stewing and pot roasting. Dry heat includes procedures in which heat is transferred to the composite material in the absence of exogenously-provided liquid. Examples of dry heat include grilling, smoking, broiling, baking, roasting, sauteing, searing and frying (e.g., deep frying). Dry heat additionally includes heating of a composite material coated in a batter or breading and fried in an oil (e.g., a vegetable oil, a seed oil, or an animal fat such as bacon fat or beef tallow).

### M. Addition of Protein Isolates

The composite material can be further processed by the addition of protein isolates, e.g., plant or animal proteins such as pea, soy, albumin, whey, casein, and faba; or pre-digested polypeptides (e.g., peptone, tryptone, or yeast extract). The protein can be provided in the form of a meal.

### VIII. Animal Flesh Analogues

Provided herein are foods products comprising analogues of animal flesh, such as meat, poultry and fish. Analogues are produced by processing composite materials to provide taste, smell, appearance and/or mouthfeel of animal products.

### A. Tuna Analogue Product

Tuna analogue has an appearance of bright, blood red to light red, opaque and soft luster appearance. The taste and mouthfeel are perceived as one or more of metallic, savory, meaty, and fatty. The aroma of the tuna analogue is aldehydic, waxy, floral, lemon, citrus, marine, fatty, orris, mushroom, earthy, and woodsy.

The taste of tuna can be provided by three different free amino acids and a 5′-nucleotide: histidine, arginine, lysine and inosine monophosphate (IMP). They are present at more than 25 times that of salmon, and contribute a savory, meaty flavor. One or more of ferrous sulfate, magnesium chloride, and/or potassium chloride can provide metallic notes to the final taste. The taste of tuna is also provided by two main organic acids that distinguish it from salmon: tartaric acid and lactic acid. Tuna taste can be provided by Maxavor M (DSM™).

The aroma of tuna is characterized by volatile odor compounds (a subset of volatile organic compounds (“VOCs”), including one or more of 3,5-Octadien-2-one, pentadecane and 2-decanone. These contribute the aromatic descriptors in the first paragraph. Other distinguishing VOCs are identified in Table 12.

The blood red to light red color can be provided by natural and artificial colors, including one or more of the following: lycopene, carmine, betacyanin, cyanidin, anthocyanins (i.e. delphinidin, pelargonidin, peonidin, petunidin, malvidin), azorubine (carmoisine), Allura red AC, Lithol rubin BK, Amaranth, red 2G, Ponceau 4R (Cochineal red A), canthaxanthin, erythrosine (FD&C Red No. 3), iron oxides, and iron hydroxides.

### B. Salmon Analogue Product

Salmon analogue can have a pinkish-orange hue with white to cream-colored fat layers.

The taste and mouthfeel are perceived as creamy, sulfury, fresh, and savory. The aroma of the salmon analogue is camphoraceous, herbal, rosemary, sage, fruity, waxy and solvent-like.

The taste of salmon is much more neutral and has fewer 5′-nucleotides (AMP, IMP, GMP) compared to tuna, leading to a decreased perception of meatiness and savoriness.

Instead, much of the flavor can be provided by VOCs from fat oxidation. These VOCs are one or more of the compounds in Table 12.

Salmon taste can be provided by Maxavor S (DSM™).

The pinkish-orange color can be provided by natural and artificial colors, including one or more of the following: beta-carotene, betaxanthins, canthaxanthin, astaxanthin, zeaxanthin, curcuminoids, crocin (Natural Yellow 6), bixin, norbixin, capsorubin, capsanthin, Tartrazine (FD&C Yellow No. 6,5), FD&C Yellow No. 6, Quinoline yellow (D&C Yellow No. 10), luteins, β-Apo-8′-carotenal, iron oxides, and iron hydroxides.

### IX. Kits

Food products as disclosed herein can be included in kits with other products. For example, a fish product can be combined with other products to form a sushi kit. The sushi kit can include a fish product as disclosed herein and one or more of, seaweed (e.g., nori), rice, soy sauce, wasabi, and chopsticks.

### X. Medical Materials

In certain embodiments, cultures comprising a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel) and human cells can be used in the manufacture of functional medical materials such as, for example, drug delivery materials, tissue grafts and tissue implants. Exemplary human cells include stem cells (e.g., mesenchymal stem cells), fibroblasts, tenoblasts, tenocytes, myoblasts, myocytes, chondroblasts, chondrocytes, osteoblasts, osteoclasts, osteocytes, and adipocytes. Methods for culturing human cells are known in the art. For example, human cells can be cultured in MEM (e.g., α-MEM) or Eagle's medium supplemented with fetal bovine serum (e.g., 10-20%), glutamine penicillin and streptomycin. Serum-free media for use in the culture of human cells are also known in the art.

### EXAMPLES

### I. Example 1: Production of a K. xylinus Scaffold

A culture of Komagataeibacter xylinus is grown in a culture system, for example, the system described in International Publication WO 2022/245683; in the culture medium shown in Table 1. Cells are grown at 27.5° C. and 60% relative humidity for 10-14 days, at which time a sheet of cellulose-containing extracellular matrix is produced. The sheet is harvested and washed in 1% (w/v) NaOH at 80-90° C. for 30-60 minutes, followed by two brief washes with distilled water. For storage, the cellulose scaffold is steam-sterilized and stored in sterile water at 4° C.

TABLE 1
Bacterial culture medium

35 g/L glucose
25 g/L fructose
2.5 g/L yeast extract
2.5 g/L peptone
5 g/L KH₂PO₄
0.1M acetate, pH 4.6

### II. Example 2: Production of a K. hansenii Scaffold

A culture of Komagataeibacter hansenii is grown in a culture system, for example, the system described in International Publication WO 2022/245683; in the culture medium shown in Table 1. Cells are grown at 27.5° C. and 60% relative humidity for 10-14 days, at which time a sheet of cellulose-containing extracellular matrix is produced. The sheet is harvested and washed in 1% (w/v) NaOH at 80-90° C. for 30-60 minutes, followed by two brief washes with distilled water. For storage, the cellulose scaffold is steam-sterilized and stored in sterile water at 4° C.

### III. Example 3: Production of a K. Rhaeticus Scaffold

A culture of Komagataeibacter rhaeticus is grown in a culture system, for example, the system described in International Publication WO 2022/245683; in the culture medium shown in Table 1. Cells are grown at 27.5° C. and 60% relative humidity for 10-14 days, at which time a sheet of cellulose-containing extracellular matrix is produced. The sheet is harvested and washed in 1% (w/v) NaOH at 80-90° C. for 30-60 minutes, followed by two brief washes with distilled water. For storage, the cellulose scaffold is steam-sterilized and stored in sterile water at 4° C.

### IV. Example 4: Production of a Composite Material Using A. oryzae

A sheet of a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel), manufactured as described in any of Examples 1-3, is placed into a culture vessel and submerged or immersed in fungal culture medium. Constituents and concentrations of the fungal culture medium are shown in Table 2.

TABLE 2
Fungal culture medium A

| Compound | Concentration | |
|---|---|---|
| sucrose | 30.0 | g/L |
| NaNO₃ | 2.0 | g/L |
| K₂HPO₄ | 1.0 | g/L |
| MgSO₄ | 0.5 | g/L |
| KCl | 0.5 | g/L |
| FeSO₄ | 0.01 | g/L |

The medium is inoculated with Aspergillus oryzae and cultured at 25° C. and 60% relative humidity for 72 hours; at which time a composite material, containing a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel), fungal cells, and fungal protein, is obtained. The composite material is removed from the culture vessel and either stored in 0.1 M sodium acetate, pH 4.5 at 4° C., or processed.

### V. Example 5: Production of a Composite Material Using F. venenatum

A sheet of a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel), manufactured as described in any of Examples 1-3, is placed into a culture vessel and submerged in fungal culture medium.

Constituents and concentrations of the fungal culture medium are shown in Table 3.

TABLE 3
Fungal culture medium B

| Compound | Concentration | |
|---|---|---|
| sucrose | 15 | g/L |
| trisodium citrate•2H₂O | 3.0 | g/L |
| KH₂PO₄ | 5.0 | g/L |
| NH₄NO₃ | 2.0 | g/L |
| MgSO₄ | 0.2 | g/L |
| CaCl₂ | 0.1 | g/L |
| biotin | 0.25 | mg/ml |
| Citric acid, monohydrate | 5 | mg/L |
| ZnSO₄•7H₂O | 5 | mg/L |
| Fe(NH₄)₂(SO₄)₂•6H₂O | 1.0 | mg/L |
| CuSO₄•5H₂O | 0.25 | mg/L |
| MnSO₄, monohydrate | 0.05 | mg/L |
| H₃BO₃, anhydrous | 0.05 | mg/L |
| NaMoO₄•2H₂O | 0.05 | mg/L |

The medium is inoculated with Fusarium venenatum and cultured at 27.5° C. and 60% relative humidity for 72 hours; at which time a composite material, containing a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel), fungal cells, and fungal protein, is obtained. The composite material is removed from the culture vessel and either stored in 0.1 M sodium acetate, pH 4.5 at 4° C., or processed.

### VI. Example 6: Production of a Composite Material Using N. intermedia

A sheet of a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel), manufactured as described in any of Examples 1-3, is placed into a culture vessel and submerged in fungal culture medium. Constituents and concentrations of the fungal culture medium are shown in Table 3.

The medium is inoculated with Neurospora intermedia and cultured at 27.5° C. and 60% relative humidity for 72 hours; at which time a composite material, containing a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel), fungal cells, and fungal protein, is obtained. The composite material is removed from the culture vessel and either stored in 0.1 M sodium acetate, pH 4.5 at 4° C., or processed.

### VII. Example 7: Adjusting the pH of the Composite Material

The pH of the composite material is adjusted, to a pH of approximately 6.5, to optimize the flavor and/or texture of the material. Depending on the starting pH of the material, pH is reduced using acetate or HCl; and pH is increased using imidazole, phosphate or bicarbonate.

### VIII. Example 8: Removal of Fungal Cells from Composite Material by Elevated Temperature

Composite material, obtained as described in either of Examples 4 or 5, is treated to kill fungal cells by immersing the material in boiling water or buffer (e.g., 0.1 m Na acetate buffer, pH 4.5) for 1 hr. Boiling is conducted at ambient pressure or at elevated pressure, e.g., by autoclaving or high-pressure pasteurization.

### IX. Example 9: Removal of Fungal Cells from Composite Material by UV Irradiation

Composite material, obtained as described in either of Examples 4 or 5, is treated to kill fungal cells. Fungal cells are killed by subjecting the material to irradiation with ultraviolet light (e.g., at wavelengths of 240 to 300 nm) for 30 min.

### X. Example 10: Removal of Fungal Cells from Composite Material Using Ionizing Irradiation

Composite material, obtained as described in either of Examples 4 or 5, is treated to kill fungal cells. Fungal cells are killed by subjecting the material to ionizing radiation (e.g., gamma rays) for 30 min.

### XI. Example 11: Shaping of Composite Material

Composite material containing a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel), fungal protein, and optionally fungal cells, is shaped by cutting or molding, using tools and methods familiar to those in the field of food processing.

### XII. Example 12: Tenderization of Composite Material

Composite material containing a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel), fungal protein, and optionally fungal cells, is mechanically tenderized by repeatedly penetrating the material with stainless steel blades, having dimensions of approximately 1 mm×5 mm, set approximately 3 mm apart.

### XIII. Example 13: Production of a Foamed K. xylinus Scaffold

A culture of Komagataeibacter xylinus is grown in a culture system, for example, the system described in International Publication WO 2022/245683; using the culture medium shown in Table 1. Prior to culture of the bacteria, the culture medium is autoclaved, then combined with the components shown in Table 4, and the mixture (supplemented culture medium) is heated until the additional components have dissolved.

TABLE 4
Emulsifier mixture A

| Compound | Concentration |
|---|---|
| xanthan gum | 1% (w/w) |
| sodium alginate | 1% (w/w) |
| locust bean gum | 1% (w/w) |
| carrageenan | 1% (w/w) |
| guar gum | 1% (w/w) |
| glyceryl monooleate | 1% (w/w) |
| glyceryl distearate | 1% (w/w) |
| dextrose | 1% (w/w) |

The supplemented culture medium is cooled to below 30° C. and inoculated with a 5% volume of a log-phase culture of K. xylinus. The inoculated culture is then subjected to shear force to generate a foam, using a homogenizer at a speed of about 1,000 rpm, until an overrun of 150-300% is obtained. The foamed culture is incubated for 10 days at 27° C., at which time a foamed cellulose sheet is produced. The sheet is harvested and washed with 1% (w/v) NaOH at 80° C. for one hour; followed by two brief washes with distilled water. If desired, the foamed cellulose scaffold is steam-sterilized and stored in sterile water at 4° C.

### XIV. Example 14: Production of a Foamed K. xylinus Scaffold

A culture of Komagataeibacter xylinus is grown in a culture system, for example, the system described in International Publication WO 2022/245683, using the culture medium shown in Table 1. Prior to culture of the bacteria, the culture medium is autoclaved, then combined with the components shown in Table 5, and the mixture (supplemented culture medium) is heated until the additional components have dissolved.

TABLE 5
Emulsifier mixture B

| Compound | Concentration |
|---|---|
| xanthan gum | 1% (w/w) |
| gelatin | 1% (w/w) |
| locust bean gum | 1% (w/w) |
| cellulose gum | 1% (w/w) |
| guar gum | 1% (w/w) |
| whey protein | 1% (w/w) |
| dextrose | 1% (w/w) |

The supplemented culture medium is cooled to below 30° C. and inoculated with a 5% volume of a log-phase culture of K. xylinus. The inoculated culture is then subjected to shear force to generate a foam, using a homogenizer at a speed of about 1,000 rpm, until an overrun of 150-300% is obtained. The foamed culture is incubated for 10 days at 27° C., at which time a foamed cellulose sheet is produced. The sheet is harvested and washed with 1% (w/v) NaOH at 80° C. for one hour; followed by two brief washes with distilled water. If desired, the foamed cellulose scaffold is steam-sterilized and stored in sterile water at 4° C.

### XV. Example 15: Production of a Composite Material Using Foamed Scaffold and A. oryzae

A sheet of foamed bacterial cellulose, manufactured as described in either of Examples 13 or 14, is placed into a culture vessel and submerged in fungal culture medium. Constituents and concentrations of the fungal culture medium are shown in Table 2.

The medium is inoculated with Aspergillus oryzae and cultured at 27° C. and 60% relative humidity for 72 hours; at which time a composite material, containing foamed bacterial cellulose, fungal cells, and fungal protein, is obtained. The composite material is removed from the culture vessel and either stored in 0.1 M sodium acetate, pH 4.5 at 4° C. or processed (as described supra Examples 11 and 12).

### XVI. Example 16: Production of a Composite Material Using Foamed Scaffold and F. venenatum

A sheet of foamed bacterial cellulose, manufactured as described in either of Examples 13 or 14, is placed into a culture vessel and submerged in fungal culture medium. Constituents and concentrations of the fungal culture medium are shown in Table 3.

The medium is inoculated with Fusarium venenatum and cultured at 27.5° C. and 60% relative humidity for 72 hours; at which time a composite material, containing foamed bacterial cellulose, fungal cells, and fungal protein, is obtained. The composite material is removed from the culture vessel and either stored in 0.1 M sodium acetate, pH 4.5 at 4° C., or processed (as described supra Examples 11 and 12).

### XVII. Example 17: Production of a Composite Material Using Foamed Scaffold and N. intermedia

A sheet of foamed bacterial cellulose, manufactured as described in either of Examples 13 or 14, is placed into a culture vessel and submerged in fungal culture medium. Constituents and concentrations of the fungal culture medium are shown in Table 3.

The medium is inoculated with Neurospora intermedia and cultured at 27.5° C. and 60% relative humidity for 72 hours; at which time a composite material, containing foamed bacterial cellulose, fungal cells, and fungal protein, is obtained. The composite material is removed from the culture vessel and either stored in 0.1 M sodium acetate, pH 4.5 at 4° C., or processed (as described supra Examples 11 and 12).

### XVIII. Example 18: Food Product: Bacterial Culture

Yeast extract, peptone, KH₂PO₄ and citric acid are dissolved in water to concentrations of 2.5 grams/liter, 2.5 g/I, 1.35 g/I and 0.75 g/I, respectively, and autoclaved. Separately, dextrose and fructose are dissolved in water to concentrations 35 g/I and 25 g/l, respectively, and filter-sterilized. After the autoclaved mixture has cooled, the sterilized sugars are added thereto and the pH of the mixture is adjusted to 4.7 to yield “bacterial culture medium.”

With the bacterial culture medium at a temperature of approximately 70-80° C., xanthan gum (0.5% w/v) and glycerol monostearate (1.5% w/v) are added to the bacterial culture medium while a shear force is being applied to the medium. After the mixture has emulsified, to form “foamed culture medium,” it is allowed to cool to below 50° C.

Frozen stock of Komagataeibacter xylinus ATCC 53582 is inoculated into bacterial culture medium (as above) and incubated at 27° C. for 24 hours. This seed culture is used to inoculate foamed culture medium, at a temperature of 50° C. or lower, at a 1% pitch rate. The inoculated foamed culture medium is cultured at 27° C. for 5-14 days.

The culture is observed and, when the scaffold produced by the bacteria attains the desired properties (e.g., size, thickness), the scaffold is removed from the fermentation vessel, separated from the medium, and washed with 1% (v/v) NaOH to remove bacterial cells. The decellularized scaffold is then boiled in sterile water until the rinse attains a pH of around 7.

### XIX. Example 19: Food Product: Fungal Culture

Yeast extract and peptone are dissolved in water to concentrations of 3.0 grams/liter and 5.0 g/l, respectively, and autoclaved. Separately, malt extract and dextrose are dissolved in water to concentrations 3.0 g/I and 10 g/l, respectively, and filter-sterilized. After the autoclaved mixture has cooled, the sterilized sugars are added thereto and the pH of the mixture is adjusted to around 7 to yield “fungal culture medium.”

Spores of Aspergillus oryzae NRRL 3485 are cultured on 1.5% agar plates of fungal culture medium at 37° C. for 72 hours. A small amount of fungal biomass is removed from the plate, inoculated into 100 ml of fungal culture medium and grown overnight at 37° C. A portion of the overnight culture is inoculated into fungal culture medium containing the decellularized scaffold of Example 18. Culture is conducted for 72 hours, at which time composite material, containing fungal biomass infiltrating a cellulose scaffold, is removed from the culture and heated at 80° C. for 20 min.

### XX. Example 20: Food Product: Marination

The following ingredients are mixed, in water, to form a marinade (all percentages are weight:volume):
2.5% yeast extract (can function as a flavorant)
0.5% NaCl (can function as a flavorant)
2% omega-3 algal oil (can function as a nutrient)
0.1% canthaxanthin (can function as a colorant)
0.25% guar gum (can function as a freeze-thaw stabilizer)
5% trehalose (can function as a freeze-thaw stabilizer)
0.1% nutrient mixture (containing equimolar amounts of dicalcium phosphate, niacinamide, calcium-D-pantothenate, pyridoxine HCl, riboflavin, potassium iodide sodium selenite, cyanocobalamin and ferrous sulfate)
0.5% CaCl₂ (can assist in cross-linking (structural integrity))
   If necessary, the mixture is gently heated to aid dissolution of the components.

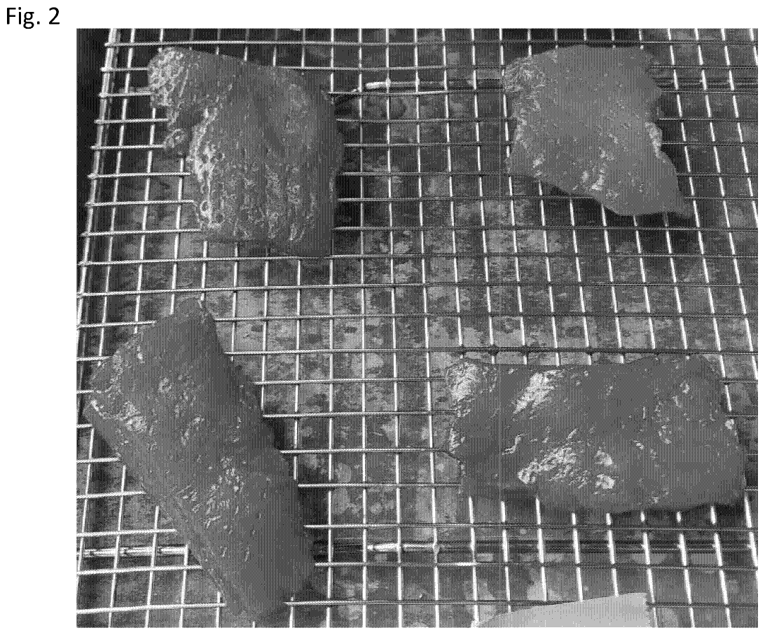

The composite material of Example 19 and the marinade solution described in the previous paragraph are placed together, at a 2:1 weight ratio, in a vacuum tumbler. Avacuum of 675 mBar is applied to the tumbler and the tumbler is rotated, at 10 rpm for 20 min. The marinated composite material is removed from the tumbler and can be stored in water at 4° C., or subjected to cutting, shaping and/or layering. Examples of marinated food products are shown in FIG. 2.

### XXI. Example 21: Food Product: Layering

A high internal phase emulsion (HIPE) is formed by mixing an aqueous phase and an oil phase. The aqueous phase contains (all percentages are weight:volume):
0.5% kappa-carrageenan
0.5% iota-carrageenan
1% sodium alginate
2% transglutaminase
96% water.

The oil phase contains:
99% algal oil
0.5% candelilla wax
0.5% glycerol monostearate.

The aqueous phase and the oil phase are separately brought to a temperature of 70° C. The aqueous phase is then subjected to rotary agitation at 1200 rpm, and the oil phase is added slowly to the aqueous phase as it is being agitated. Agitation at 1200 rpm is continued until the emulsion reaches a temperature of 30° C.

A 10 mm thick sheet of marinated composite material, as described in Example 20, is placed on a surface. A layer of HIPE, 1-2 mm thick, is applied to the sheet using a spreader nozzle. A second 10 mm sheet of composite material is placed atop the layer of HIPE, and a second layer of HIPE is applied atop the second sheet. Finally, a third 10 mm sheet of composite material is placed above the second layer of HIPE. Additional alternating layers of HIPE and composite material can be constructed as desired.

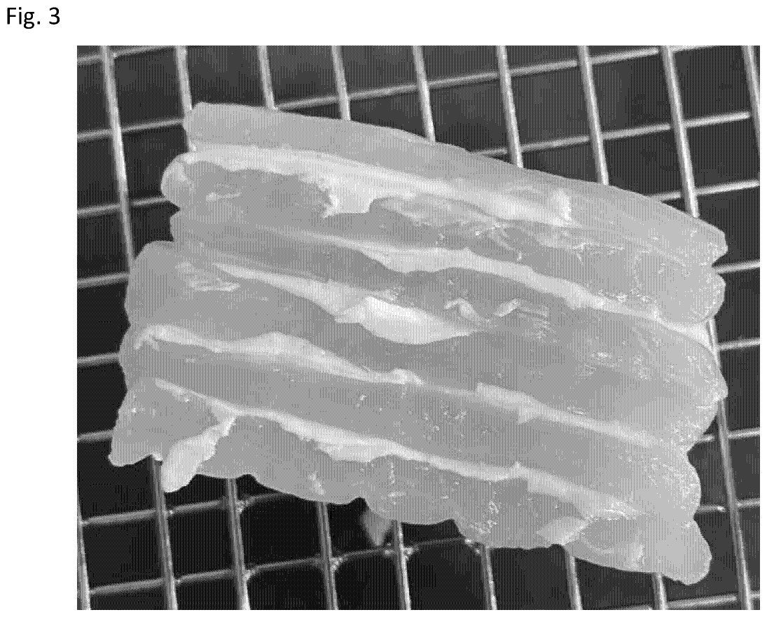

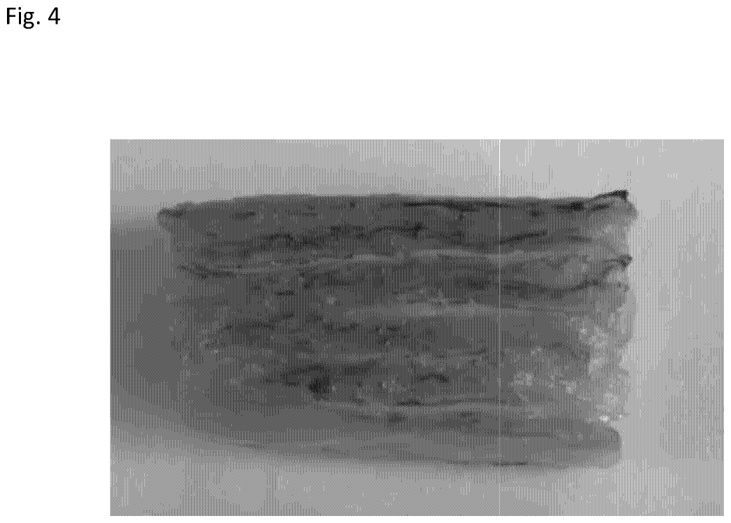

An exemplary layered composite material (raw) is shown in FIG. 3. An exemplary layered composite material (sauteed) is shown in FIG. 4.

### XXII. Example 22: Comparative Storage Modulus of Composite Material

Composite material was made by the method of Example 4, and its storage modulus was determined and compared to the storage modulus of tuna and calamari. The results are shown in Table 6.

TABLE 6

| | | | COMPOSITE | | | |
|---|---|---|---|---|---|---|
| | | | MATERIAL | | | |
| Strain % | CALAMARI | STDEV | PRODUCT | STDEV | TUNA | STDEV |
| 0.1 | 41627.4333 | 11194.7676 | 110532.44 | 15181.3781 | 31181.8833 | 6917.38462 |
| 0.158 | 41627.2167 | 11124.8435 | 111129.8 | 14367.3836 | 31211.9833 | 7206.86105 |
| 0.251 | 41390.9167 | 10983.6121 | 110188.04 | 13565.2193 | 30814.7667 | 7002.02578 |
| 0.398 | 40789.05 | 10685.2626 | 106194.66 | 12096.067 | 30285.9833 | 6815.71654 |
| 0.631 | 39595.15 | 10175.3344 | 99329.74 | 10965.564 | 29320.9 | 6566.95738 |
| 1 | 37469.5333 | 9380.87868 | 90691.32 | 9889.04565 | 27830.4 | 6230.49025 |
| 1.58 | 34190 | 8320.99335 | 80910.76 | 8509.04219 | 25916.3833 | 5763.6978 |
| 2.51 | 29641.2 | 7054.59792 | 70851.36 | 7758.09995 | 23497.3833 | 5146.76491 |
| 3.98 | 24062.2 | 5664.8281 | 58886.74 | 8494.4058 | 20760.5167 | 4537.56972 |
| 6.31 | 18475.3 | 4569.22015 | 48121.24 | 9899.70497 | 17878.8167 | 3915.76801 |
| 10 | 13709.8 | 3846.47276 | 39119.42 | 9046.23959 | 15042.2567 | 3327.46353 |
| 15.8 | 10241.8917 | 3251.53911 | 29442.78 | 5559.313 | 12384.0583 | 2769.55266 |
| 25.1 | 7904.30167 | 3091.53558 | 21042.4 | 2221.16752 | 9898.51 | 2224.2948 |
| 39.8 | 6454.67667 | 3242.65397 | 16592.16 | 981.948093 | 7812.41167 | 1724.13761 |
| 63.1 | 5427.19833 | 3559.16943 | 12786.88 | 1693.12933 | 5964.245 | 1259.37755 |
| 100 | 4392.245 | 3690.84638 | 9697.582 | 1902.50943 | 4286.385 | 818.349288 |

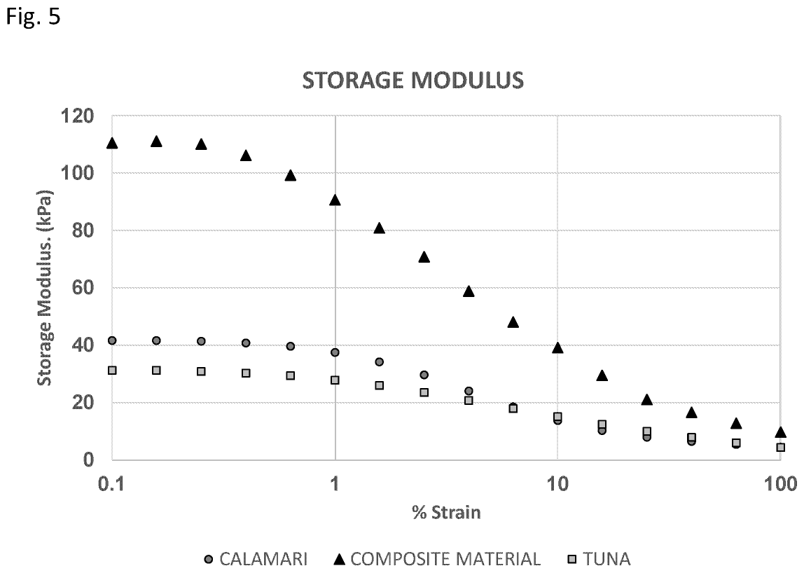

The results are summarized in FIG. 5.

### XXIII. Example 23: Comparative Loss Modulus of Composite Material

Composite material was made by the method of Example 4, and its loss modulus was determined and compared to the loss modulus of tuna and calamari. The results are shown in Table 7.

TABLE 7

| | | | COMPOSITE | | | |
|---|---|---|---|---|---|---|
| | | | MATERIAL | | | |
| Strain % | CALAMARI | STDEV | PRODUCT | STDEV | TUNA | STDEV |
| 0.1 | 7653.11667 | 1747.30978 | 42695.18 | 4467.26197 | 6600.74833 | 1743.31216 |
| 0.158 | 7746.02833 | 1754.13899 | 39814.58 | 3502.28875 | 6617.2 | 1577.944 |
| 0.251 | 7791.39167 | 1792.15665 | 38812.44 | 3634.11317 | 6534.19 | 1650.03753 |
| 0.398 | 7842.54833 | 1852.39155 | 38165.64 | 3588.61514 | 6487.65333 | 1615.0863 |
| 0.631 | 7905.12167 | 1908.19442 | 37299.54 | 3264.27966 | 6483.44667 | 1599.01996 |
| 1 | 7955.32333 | 1940.2686 | 35986.92 | 3015.1086 | 6453.075 | 1591.49798 |
| 1.58 | 7914.68 | 1935.22962 | 34057.6 | 2936.54066 | 6335.51833 | 1526.27684 |
| 2.51 | 7731.85 | 1885.62737 | 31453.52 | 2787.95877 | 6142.76333 | 1456.14611 |
| 3.98 | 7347.79833 | 1781.89414 | 27358.44 | 2885.80502 | 5811.46667 | 1348.97191 |
| 6.31 | 6769.44667 | 1675.97384 | 23601.12 | 2965.9285 | 5325.955 | 1206.21806 |
| 10 | 6010.695 | 1546.85037 | 19849.6 | 3004.63261 | 4726.01333 | 1049.7336 |
| 15.8 | 5180.94667 | 1258.04308 | 15986.76 | 2398.41142 | 4067.13833 | 884.551249 |
| 25.1 | 4375.76167 | 1022.9586 | 11752.02 | 1278.40826 | 3377.16333 | 708.759278 |
| 39.8 | 3678.52333 | 897.099221 | 9114.812 | 1112.28074 | 2785.57 | 567.642359 |
| 63.1 | 3059.675 | 926.042397 | 6873.424 | 921.553356 | 2269.05833 | 437.322125 |
| 100 | 2547.175 | 1012.86283 | 5154.818 | 830.611352 | 1826.42833 | 318.34947 |

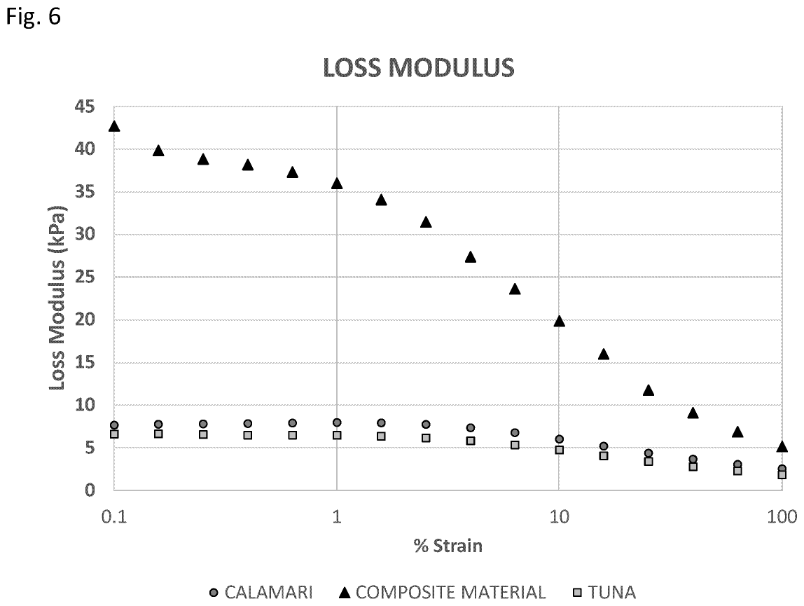

The results are summarized in FIG. 6.

### XXIV. Example 24: Comparison of the Microstructures of Composite Material and Seafood

Microstructural analysis was performed to evaluate features of bacterial cellulose (bacterial cellulose) scaffold such as size and alignment of fibers, and to compare these features to those of various native seafood species.

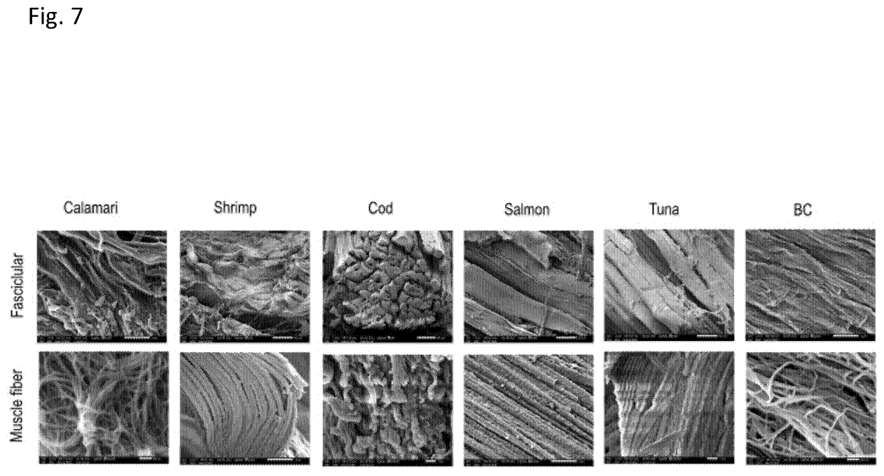

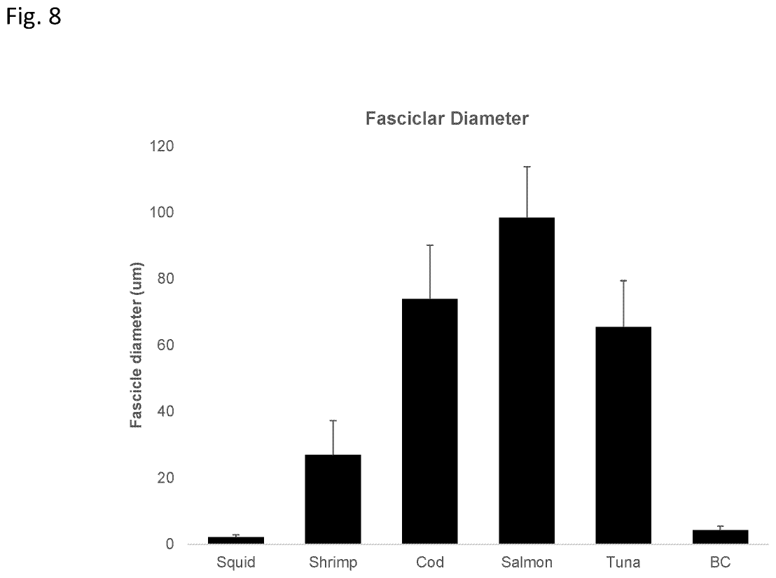

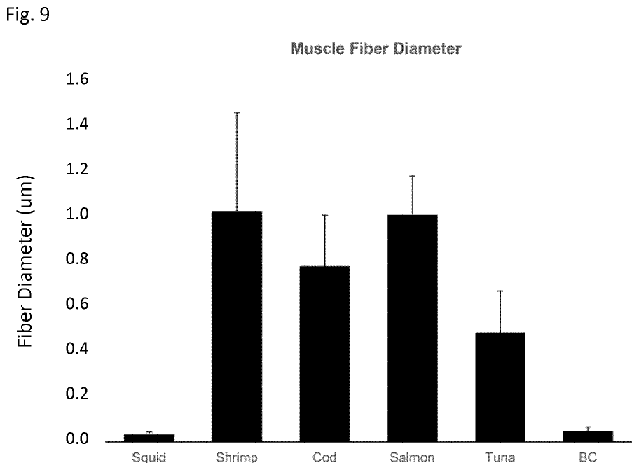

To this end, scanning electron microscope (SEM) images of several different types of seafood were obtained and compared to SEM images of bacterial cellulose scaffold made according to the method of Example 4; as shown in FIG. 7. Similar morphological features, including size and hierarchical fascicular arrangement, were observed among the different seafood specimens. The micrographs also revealed a layered structure of the bacterial cellulose scaffold, resembling the fascicular alignment observed in the seafood samples. Quantification of fascicular diameter (FIG. 8, Table 8) and fiber diameter (FIG. 9, Table 8) indicated that the widths of bacterial cellulose layers and fibers were similar to those of calamari (squid) fascicles and fibers, respectively. Further, bacterial cellulose microfibers were observed to have a random arrangement, similar to the arrangement of muscle fibers in calamari. Microfiber diameter of bacterial cellulose was also determined to be within the same order of magnitude as that of other seafood species including shrimp, cod, salmon, and tuna.

TABLE 8
Comparison of muscle fiber diameter and fascicular diameter of different
seafood muscle fibers and bacterial cellulose scaffold

| Source material | Fascicular diameter (μm) | Fiber diameter (μm) |
|---|---|---|
| Squid (calamari) | 2 | <0.1 |
| Shrimp | 22 | 1 |
| Cod | 75 | 0.7 |
| Salmon | 92 | 1 |
| Tuna | 60 | 0.4 |
| bacterial cellulose | 3 | <0.1 |

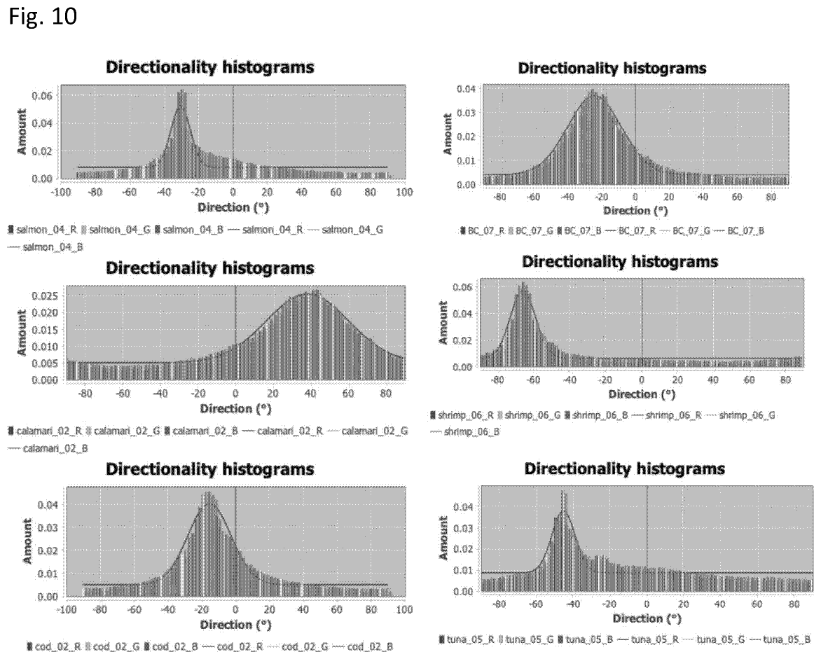

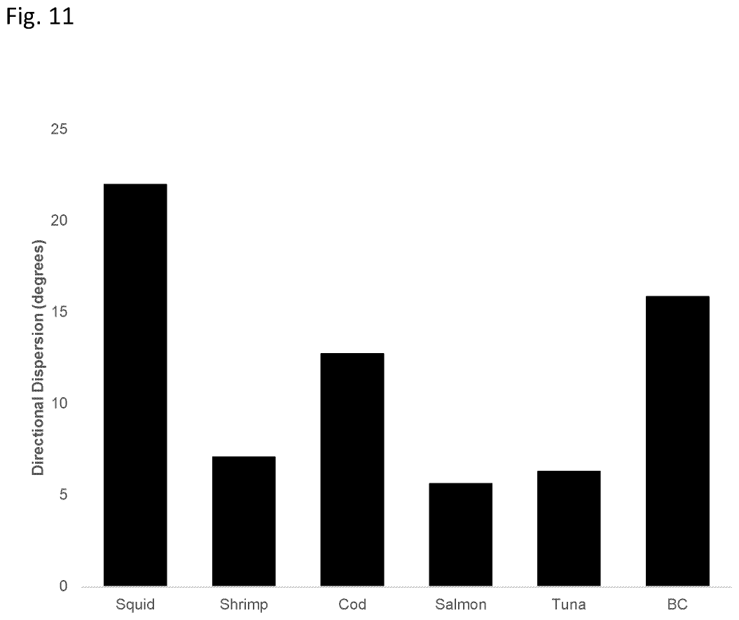

In addition to fiber size and arrangement, scaffold alignment was quantified, using scanning electron microscopy, to determine directional dispersion of bacterial cellulose fibers compared to that of various seafood species. As shown in FIG. 10, all samples had a unimodal distribution of fiber orientation, with angular dispersions between 5° and 25°. Dispersion values, shown in FIG. 11, were obtained from the width of each sample peak. Bacterial cellulose scaffold fibers had a peak directional dispersion of approximately 15.58°, whereas calamari (squid) and salmon were shown to have the greatest and least directional dispersion of approximately 21.98° and 5.62°, respectively.

### XXV. Example 25: Comparison of Mechanical Properties of Composite Material and Seafood: Tensile Strength

Bacterial cellulose scaffolds have tunable mechanical properties that can be modulated by selective strain choice and cultivation time. Ebrahimi et al. (2017) J. Chem. Engineering of Japan 50(11):857-861. With a range of mechanical tunability that can be used to mimic tissue stiffness (Table 9), bacterial cellulose has been demonstrated to be suitable for both hard and soft tissue applications including osteogenesis and differentiation of pluripotent stem cells into neuronal cells.

This work demonstrates the utility of bacterial cellulose scaffolds for use in various alternative seafood products. Current bacterial cellulose growth conditions produce scaffolds that mimic the mechanical properties of various seafood species (e.g., salmon, cod, shrimp) and can be further tuned for stiffer or softer species such as calamari and tuna by modulating bacterial culture conditions and subsequent processing of the composite materials made from the bacterial cellulose.

TABLE 9

| Sample | Tensile strength (kPa) |
|---|---|
| bacterial cellulose Scaffold | 19.1 +/− 5.1 |
| bacterial cellulose Bandage | 59 |
| bacterial cellulose/PGS Composite | 1210 +/− 292.5 |
| Shrimp | 14.7 +/− 4.2 |
| Cod | 17.2 +/− 6.2 |
| Salmon | 11.8 +/− 6.7 |
| Calamari | 89 +/− 28.2 |
| Tuna | 7 +/− 1.47 |
| Human skin tissue | 83300 +/− 34900 |
| Human brain - white and grey matter | 1.89 +/− 0.59 |
| bacterial cellulose Scaffold for human bone | 250 +/− 140 |

### XXVI. Example 26: Comparison of Mechanical Properties of Composite Material and Seafood: Cutting Force

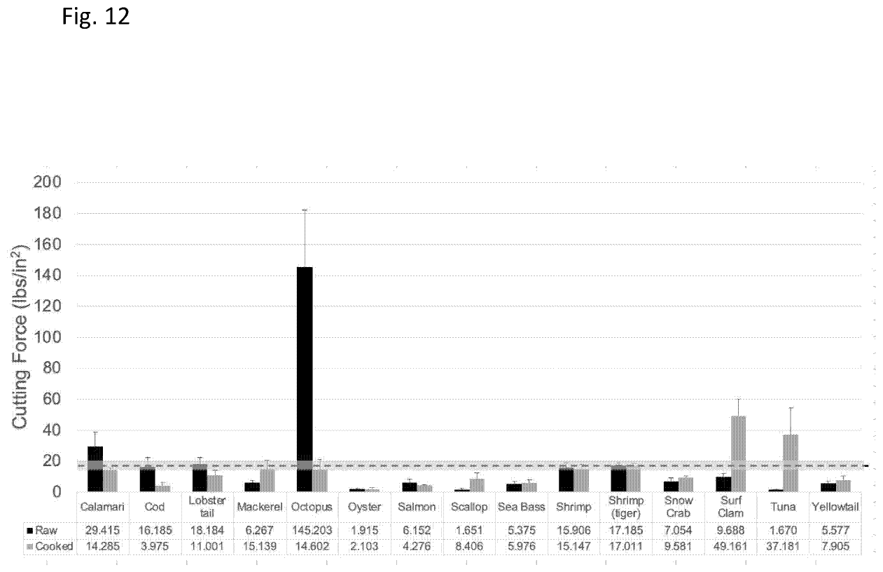

FIG. 12 and Table 10 show the cutting forces of a number of seafood products, both cooked and raw, compared with the cutting force of one embodiment of a bacterial cellulose scaffold made by the method of Example 4. It can be seen, from FIG. 12, that the value of the cutting force of the bacterial cellulose scaffold is close to those of a number of seafoods, but in some case slightly higher. By including porogens and/or emulsifiers in the bacterial culture, the cutting force of the bacterial cellulose scaffold can be reduced, up to 92%, so as to be comparable to the cutting force of a number of naturally-occurring seafoods. See FIG. 13 and Table 11.

TABLE 10
Cutting force of muscle fibers of different aquatic species (psi)

| Species | Raw | Cooked |
|---|---|---|
| Calamari | 29.415 +/− 9.308 | 14.285 +/− 1.486 |
| Cod | 16.185 +/− 6.284 | 3.975 +/− 2.406 |
| Lobster tail | 18.184 +/− 4.140 | 11.001 +/− 2.958 |
| Mackerel | 6.267 +/− 1.424 | 15.139 +/− 5.666 |
| Octopus | 145.203 +/− 37.146 | 14.602 +/− 6.509 |
| Oyster | 1.915 +/− 0.597 | 2.103 +/− 0.917 |
| Salmon | 6.152 +/− 2.094 | 4.276 +/− 0.596 |
| Scallop | 1.651 +/− 0.994 | 8.406 +/− 3.886 |
| Sea Bass | 5.375 +/− 1.941 | 5.976 +/− 1.919 |
| Shrimp | 15.906 +/− 3.320 | 15.147 +/− 2.525 |
| Snow Crab | 7.054 +/− 2.072 | 9.581 +/− 1.012 |
| Surf Clam | 9.688 +/− 2.525 | 49.161 +/− 11.198 |
| Shrimp (tiger) | 17.185 +/− 2.043 | 17.001 +/− 1.745 |
| Tuna | 1.670 +/− 0.157 | 37.181 +/− 17.235 |
| Yellowtail | 5.577 +/− 1.477 | 7.905 +/− 2.343 |

In addition, different values of cutting force can be obtained using different strains of Komagataeibacter, as shown in Table 12.

TABLE 11
Cutting forces of bacterial cellulose scaffolds
produced under various conditions

| | Avg. Cutting | |
|---|---|---|
| Culture conditions | force (lb/in²) | Std. dev. |
| K. xylinus NRRL B3780 | 166.2 | 25.3 |
| K. xylinus ATCC 53582 | 159.3 | 47.7 |
| K. xylinus ATCC 53582 Porogen templated 1:1 | 125.4 | 34 |
| K. xylinus ATCC 53582 Porogen templated 2:1 | 31.4 | 6.7 |
| K. xylinus NRRL B378 foamed* | 14.2 | 4.7 |
| K. xylinus ATCC 53582 foamed* | 13.5 | 6.1 |
| K. xylinus ATCC 700178 | 100.7 | 33.6 |
| K. xylinus ATCC 11142 | 344.3 | 180.0 |
| K. hansenii ATCC 23769 | 140.3 | 27.7 |
| K. hansenii NBRC 105051 | 32.8 | 22.4 |
| K. xylinus NBRC 13693 | 22.2 | |
| K. xylinus ACF LGCY | 19.5 | 6.2 |

*Foamed cultures contained 0.5% xanthan gum and 1.5% glycerol monostearate

### XXVII. Example 27: Freeze-Thaw Stability of Composite Material

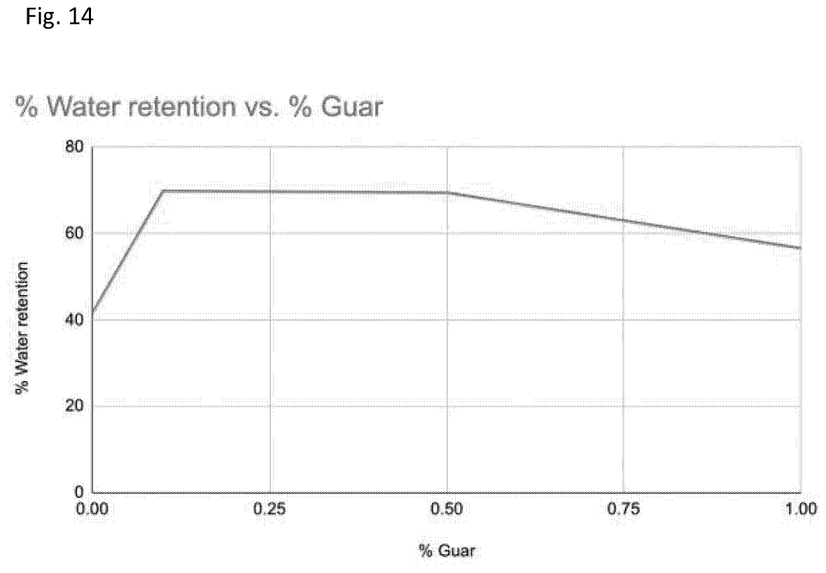

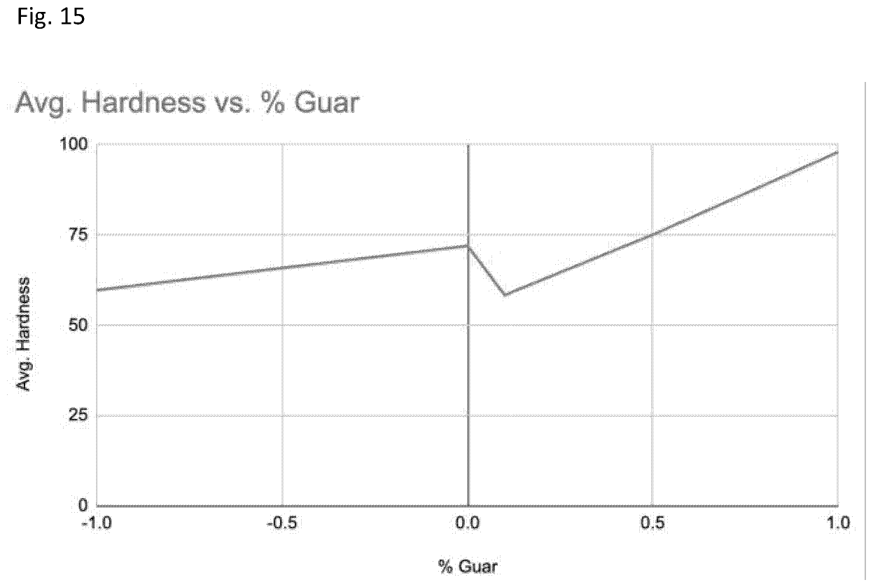

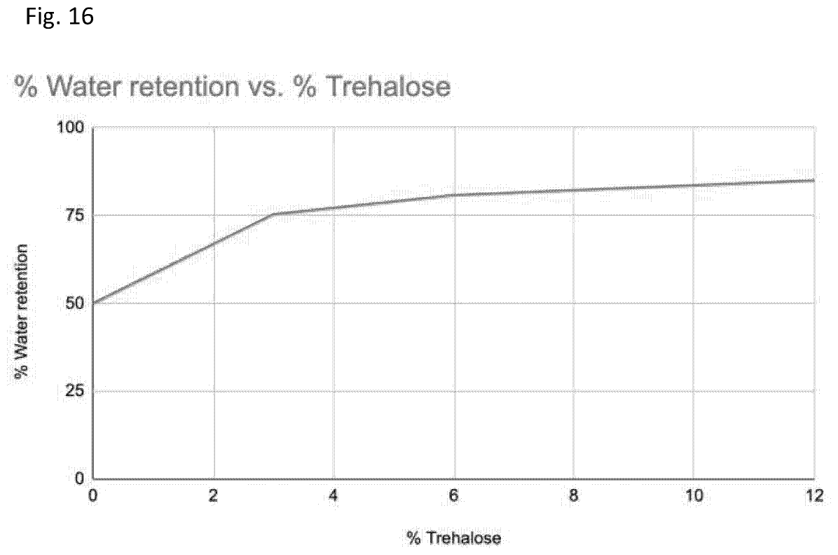

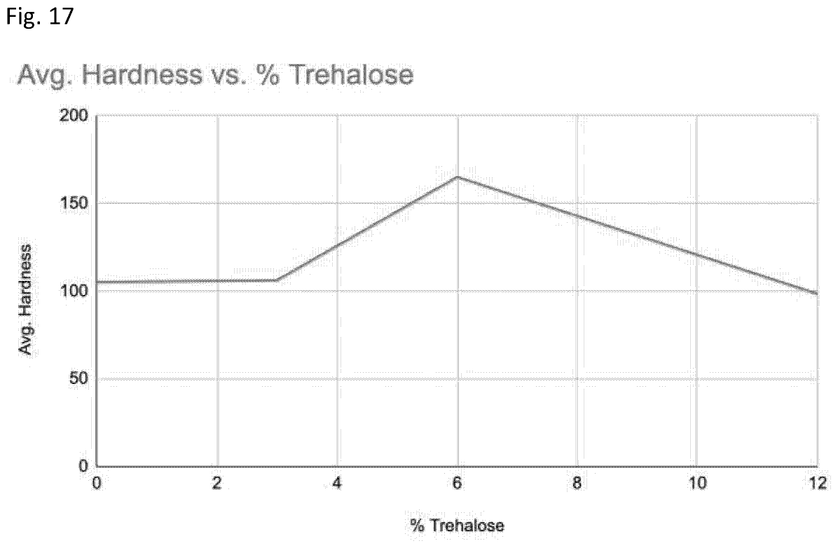

Various agents can be added to composite material, either during or subsequent to its production, to modulate its hardness and/or water retention, both of which will affect the freeze-thaw stability of the composite material. FIG. 14 shows the effect of guar gum, added during marination, on percent water retention of composite material, and FIG. 15 shows the effect of guar gum, added during marination, on average hardness of the composite material. FIG. 16 shows the effect of trehalose, added during marination, on percent water retention of composite material, and FIG. 17 shows the effect of trehalose, added during marination, on average hardness of the composite material. These results indicate that composite material can retain 70-95% of its water while also retaining the mechanical properties of various types of seafood.

### XXVIII. Example 28: Pore Size and Pore Density of Composite Material

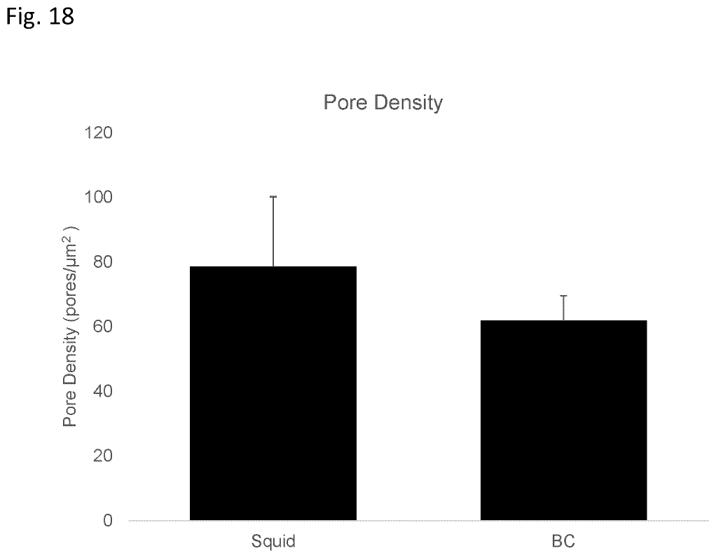

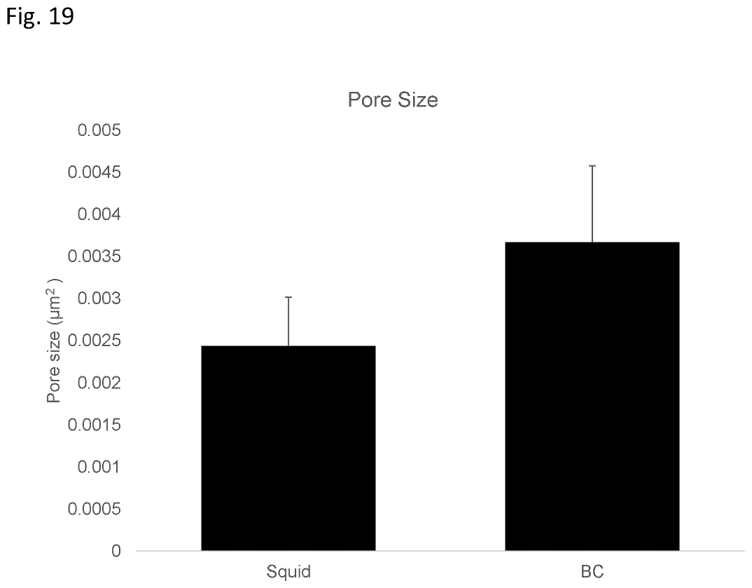

Bacterial cellulose scaffold was produced by K. xylinus strain ATCC 53582 grown in Hestrin-Schramm medium. The size and distribution of pores in this scaffold were determined (from scanning electron microscope images such as those shown in FIG. 7) and compared to those of squid (calamari) muscle fibers, using the image processing software, imagej. FIG. 18 shows a comparison of pore density, and FIG. 19 shows a comparison of pore size. It can be seen that both the size and the density of pores is similar in squid muscle fibers and bacterial cellulose scaffold.

### XXIX. Example 29: Food Product: Simulated Tuna and Salmon

Marination can be used to modulate the flavor and appearance of composite materials to resemble different types of seafood. To this end, various compounds; such as volatile organic compounds, amino acids, nucleotides and organic acids; can be added to the marinade solution. Tables 13-16 list the identity and amounts of compounds that can be added to a polymer scaffold (e.g., bacterial cellulose, a chitosan-alginate hydrogel, an alginate-gelatin polymer, cellulose acetate fibers, cellulose acetate-chitosan fibers, an agarose hydrogel, or an agarose-alginate hydrogel)/fungal protein composite material to impart the color and flavor of tuna and salmon.

TABLE 13
Volatile organic compounds (VOCs) for use in flavoring

| | | Tuna | Salmon | Scallop | Shrimp |
|---|---|---|---|---|---|
| Voc | Flavor notes | (μg/100 g) | (μg/100 g) | (ug/100 g) | (ug/100 g) |
| Hexanal | cut grass, fruity, | 37.07 | 380.74 | 32.5 | 0.598 |
| | plastic | | | | |
| Octanal | Aldehydic, waxy, | 39.02 | | 20.4 | |
| | citrus, orange peel, | | | | |
| | green, herbal, fresh, | | | | |
| | fatty | | | | |
| Nonal | waxy aldehydic rose | 74.50 | | 2.1 | 3.282 |
| | fresh orris orange | | | | |
| | peel fatty peely | | | | |
| o-xylene | cooked vegetable | | 117.66 | | |
| Z-4-heptenal | cooked vegetable, | | 673.37 | 7.53 | |
| | fishy | | | | |
| Methional | cooked potato | | 670.53 | 7.07 | |
| benzaldehyde | fruity, floral | | 547.53 | | |
| 1-octen-3-ol | mushroom | 17.50 | 186.72 | 3.26 | |
| Phenol | phenolic, sulfury, | | 789.25 | | |
| | leather | | | | |
| benzyl alcohol | herbaceous, wet | | 200.43 | | |
| | wood, floral | | | | |
| benzeneacetylaldehyde | moss, spicy | | 446.42 | 96.2 | |
| E-2-octenal | moldy, pungent, | | 937.70 | | |
| | cucumber/moss | | | | |
| menthatriene | Oily, | | 813.10 | | |
| | chemical, cooling, | | | | |
| | woody, pine, | | | | |
| | camphoreous, | | | | |
| | thyme, herbal, | | | | |
| | tropical | | | | |
| (E,Z)-2,6-nonadienal | cucumber, floral | | 525.63 | | |
| (E)-2-decenal | cooked, plastic | 2.81 | 370.42 | | |
| 2-undecanone | nutty, green, fruity | | 208.79 | 2.39 | |
| (E,Z)-2,4-decadienal | fishy, medicinal | | 349.06 | | |
| 1-methylnaphthalene | vegetal, cooked, | | 458.17 | 0.394 | |
| | green | | | | |
| (E,E)-2,4-decadienal | cooked, oily, solvent | | 817.38 | | |
| 2-methylnaphthalene | marine, green, | | 309.90 | 0.862 | |
| | solvent | | | | |
| tetradecanal | wet wood, marine, | 13.56 | 369.71 | | |
| | plastic | | | | |
| 2-Undecanone | waxy, fruity, creamy, | 21.70 | | 2.39 | |
| | fatty, orris, floral | | | | |
| pentadecane | waxy | 104.00 | | 1.25 | |
| 2,6,10,14- | | 102 | | 1.61 | |
| tetramethylpentadecane | | | | | |
| (pristane) | | | | | |
| heptadecane | | 42.37 | | | |
| 3,5-Octadien-2-one* | fruity, fatty, | 7.52 | | | 0.992 |
| | mushroom | | | | |
| (E)-2-phenyl-2-butenal | Green vegetable, | | | | 2400 |
| | floral, cocoa, nutty | | | | |
| Dodecanoic acid | Fatty, waxy | | | | 7.333 |
| 2,4-dibromophenol | | | | | 0.029 |
| 4-bromophenol | | | | | 0.02 |

Legend: The first column lists VOCs that can be used as flavoring agents for composite materials. The second column lists the flavor(s) provided by each VOC. The third column provides amounts of each VOC used in a composite material that simulates tuna. The fourth column provides amounts of each VOC used in a composite material that simulates salmon.
*3,5-Octadien-2-one was only detected in raw fish samples and was produced from α-linolenic acid degradation

TABLE 14
Flavor compounds: amino acids

| Amino acid | Tuna (mg/100 g) | Salmon (mg/100 g) |
|---|---|---|
| Asp | 3.17 | 2.5 |
| Glu | 3.54 | 24.0 |
| Thr | 2.47 | |
| Ser | 2.25 | |
| Gly | 2.66 | 19.5 |
| Ala | 8.12 | 35 |
| Met | 1.07 | 0.05 |
| Tyr | 2.03 | 5.3 |
| Lys | 188.98 | 7.5 |
| Arg | 135.58 | 2.7 |
| Val | 2.2 | |
| Ile | 1.15 | 4.2 |
| Leu | 2.24 | 6.95 |
| Phe | 1.14 | 3.95 |
| His | 437.08 | 10.1 |
| Cys | | 0.07 |

Legend: The first column of the table lists various amino acids that can be used to flavor composite materials. The second column lists the amounts of these compounds that are used in a composite material that simulates tuna. The third column lists the amounts of these compounds that are used in a composite material that simulates salmon.

TABLE 15
Flavor compounds: nucleotides

| | Tuna | Salmon |
|---|---|---|
| Nucleotide | (mg/100 g) | (mg/100 g) |
| adenosine monophosphate (AMP) | 1.4 | ND |
| inosine monophosphate (IMP) | 342.93 | 50 |
| guanosine monophosphate (GMP) | 13.9 | |

Legend: The first column of the table lists various nucleotides that can be used to flavor composite materials. The second column lists the amounts of these compounds that are used in a composite material that simulates tuna. The third column lists the amounts of these compounds that are used in a composite material that simulates salmon.

TABLE 16
Flavor compounds: organic acids

| Organic Acid | Tuna (mg/100 g final) |
|---|---|
| Tartaric acid | 3,978.22 |
| Malic acid | 303.51 |
| Lactic acid | 4,978.01 |
| Succinic acid | 1,580.16 |

Legend: The first column of the table lists various organic acids that can be used to flavor composite materials. The second column lists the amounts of these compounds that are used in a composite material that simulates tuna.

### XXX. Example 30: An Exemplary Protocol to Produce A Food Product

Komagataeibacter bacteria and Aspergillus fungi are grown in starter cultures to serve as inocula.

A plastic tray having dimensions about 27 cm (L)×20 cm (W)×15 cm (H) is filled to a depth of about 4 cm with culture medium. The culture medium includes:
35 g/L glucose
25 g/L fructose
2.5 g/L yeast extract
2.5 g/L peptone
5 g/L KH2PO₄
0.1 M acetate, pH 4.6

In a one-step culture method, the culture medium inoculated with both the bacteria and the fungus. In a two culture method, only the bacteria is used. The culture is incubated at about 28 degrees centigrade and 60% relative humidity. The culture is also sparred with air via a two inserted into the bottom of the tank. The culture is fermented for about 14 days. At this point there is little further growth of the pellicle. The pellicle has a thickness of about 5 mm.

The pellicle is harvested by pouring out the contents of the tray to separate the pellicle from the spent culture medium. The pellicle is boiled in 0.5 molar citric acid for about 30 minutes to one hour to kill the microorganisms. These treatments improve the tenderness of the pellicle. The pellicle may be punctured at this stage to improve penetration and tenderness.

In a two culture method the pellicle is washed and immersed in a tray comprising culture medium inoculated with fungal cells. The culture is allowed to grow for 14 days to allow the fungal cells to populate voids in the scaffold.

The composite material may now be cut into desired sizes and shapes, and marinated to color and flavor the product. Flavoring will typically involve at least three elements that provide a fish-like flavor these include, salt, trimethyl amine (TMA) and sugar. Marinating is performed for 1 hour.

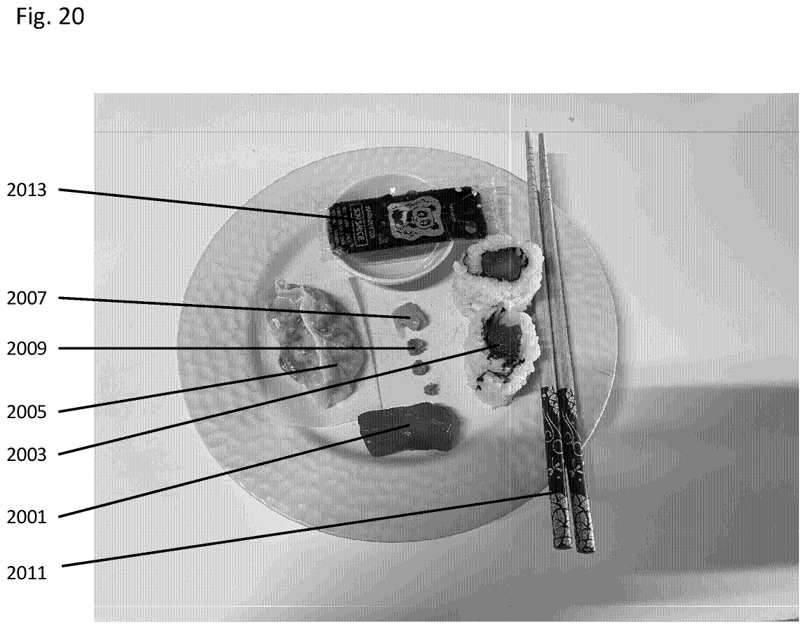

The product is now formed into sushi. A piece of the artificial fish is rolled with seaweed and rice flavored with rice vinegar, salt and sugar. The sushi roll is served with soy sauce, and wasabi. (See, e.g., FIG. 20.)

To produce a ground shrimp product, the artificial fish product is put through a meatgrinder to grind product. Then it is used to stuff a dumpling.

### Exemplary Embodiments

It is to be appreciated that certain features of the disclosure which are, for clarity, described herein in the context of separate embodiments, may also be provided in combination in a single embodiment. That is, unless obviously incompatible or specifically excluded, each individual embodiment is deemed to be combinable with any other embodiment(s) and such a combination is considered to be another embodiment. Conversely, various features of the disclosure that are, for brevity, described in the context of a single embodiment, may also be provided separately or in any sub-combination. Finally, while an embodiment may be described as part of a series of steps or part of a more general structure, each said step may also be considered an independent embodiment in itself, combinable with others.

The inventions disclosed herein are exemplified by the following embodiments.

1. A method comprising:
(a) providing a scaffold comprising bacterial cellulose; and
(b) populating the scaffold with eukaryotic cells, to produce a composite material.

2. The method of embodiment 1, wherein the scaffold is free or essentially free of living or dead bacterial cells.

3. The method of embodiment 1, wherein the eukaryotic cells comprise a fungal cell, a plant cell or an animal cell.

4. The method of embodiment 1, wherein the eukaryotic cells comprise an animal cell selected from a vertebrate cell, a chordate cell, an echinoderm cell, a crustacean cell, or a molluscan cell.

5. The method of embodiment 1, wherein the eukaryotic cells comprise a vertebrate cell selected from a primate cell, a mammalian cell, an avian cell, a reptilian cell, an amphibian cell, and a piscine cell.

6. The method of embodiment 1, wherein the eukaryotic cells comprise a piscine cell selected from the group consisting of tuna, salmon, yellowtail, flounder, halibut, shad, mackerel, sea bass, porgy, and snapper.

7. The method of embodiment 1, wherein the eukaryotic cells comprise a molluscan cell selected from the group consisting of clam, mussel, oyster, scallop, abalone, squid and octopus.

8. The method of embodiment 1, wherein the eukaryotic cells comprise a crustacean cell selected from the group consisting of shrimp, crab and lobster.

9. The method of embodiment 1, wherein the eukaryotic cells comprise a sea urchin cell.

10. The method of embodiment 1, wherein the eukaryotic cells comprise a human cell selected from a human stem cell, a chondrocyte, a chondroblast, a tenocyte, a tenoblast, a myoblast or a myocyte.

11. The method of embodiment 1, wherein the eukaryotic cells do not comprise human cells.

12. The method of embodiment 1, wherein (a) providing the scaffold comprises:
(i) growing cellulose-producing bacterial cells in a first culture medium to produce the scaffold comprising bacterial cellulose; and
(ii) removing the first culture medium and the bacterial cells from the scaffold.

13. The method of embodiment 12, comprising converting the first culture medium to a foam before growing the bacteria.

14. The method of embodiment 12, wherein the first culture medium further comprises a emulsifier.

15. The method of embodiment 13, wherein the foam is an open-cell foam.

16. The method of embodiment 13, wherein the foam is a closed-cell foam.

17. The method of embodiment 14, wherein the emulsifier is selected from one or more of a monoglyceride, a diglyceride, a polyglycerol ester such as glycerol monostearate or glycerol monooleate, lecithin, polysorbate, a phospholipid, a glycolipid, and a glycoprotein.

18. The method of embodiment 17, wherein the emulsifier comprises 1, 2, 3, 4, 5, 6, or 7 elements selected from:
(a) xanthan gum at a concentration of 0.2 to 2.0 weight percent;
(b) sodium alginate at a concentration of 0.2 to 2.0 weight percent;
(c) locust bean gum at a concentration of 0.2 to 2.0 weight percent;
(d) carrageenan at a concentration of 0.2 to 2.0 weight percent;
(e) guar gum at a concentration of 0.2 to 2.0 weight percent;
(f) a monoglyceride at a concentration of 0.2 to 2.0 weight percent; and
(g) a diglyceride at a concentration of 0.2 to 2.0 weight percent.

19. The method of embodiment 17, wherein the emulsifier comprises 1, 2, 3, 4, 5, or 6 elements selected from:
(a) xanthan gum at a concentration of 0.2 to 2.0 weight percent;
(b) gelatin at a concentration of 0.2 to 2.0 weight percent;
(c) locust bean gum at a concentration of 0.2 to 2.0 weight percent;
(d) cellulose gum at a concentration of 0.2 to 2.0 weight percent;
(e) guar gum at a concentration of 0.2 to 2.0 weight percent; and
(f) whey protein concentrate at a concentration of 0.2 to 2.0 weight percent.

20. The method of embodiment 14, further comprising adding a gelling agent.

21. The method of embodiment 20, wherein the gelling agent is selected from xanthan gum, gelatin, sodium alginate, locust bean gum, cellulose gum, carrageenan, guar gum, whey protein concentrate, dextrose, a sugar, methylcellulose, carboxymethylcellulose, and hydroxypropyl methylcellulose.

22. The method of any of embodiments 20-21, wherein the gelling agent comprises a monoglyceride selected from one or more of glycerol monooleate and glycerol monostearate.

23. The method of any of embodiments 20-21, wherein the gelling agent comprises a diglyceride selected from one or more of glyceryl distearate, glyceryl dioleate, and glyceryl dicaprylate.

24. The method of any of embodiments 13-23, wherein a shear force is applied to the first culture medium.

25. The method of embodiment 24, wherein the shear force is applied using a homogenizer, e.g., at a speed of about 1,000 rpm.

26. The method of embodiment 24, wherein the foam has an overrun of 150-300%.

27. The method of any of embodiments 24-26, wherein prior to application of the shear force, the first culture medium is inoculated with a bacterial seed inoculum of 1-10% v/v to form a bacterial culture.

28. The method of embodiment 24, wherein the bacterial culture is incubated at between 20°-30° C.

29. The method of embodiment 24, wherein, the bacterial culture is incubated at about 27° C.

30. The method of any of embodiments 27-29, wherein the culture is incubated for between 5 and 14 days.

31. The method of any of embodiments 1-30, wherein in step (b), populating the scaffold comprises growing the eukaryotic cells in a vessel comprising a second culture medium and the scaffold.

32. The method of embodiment 31, wherein a cell inoculum of 0.1%-85% percent of the volume of the second culture medium is introduced into the container.

33. The method of any of embodiments 12-32, comprising producing air into the culture medium.

34. The method of any of embodiments 1-33, wherein the scaffold has a porosity of 1%-50%.

35. The method of any of embodiments 12-34, wherein the cellulose-producing bacterial cells comprise one or more bacteria selected from Acetobacter, Bacillus, Bifidobacterium, Brachybacterium, Brevibacterium, Carnobacterium, Corynebacterium, Enterococcus, Gluconobacter, Gluconacetobacter, Corynebacterium, Halomonas, Komagataeibacter, Lactobacillus, Lactococcus, Leuconostoc, Macrococcus, Microbacterium, Micrococcus, Oenocuccus, Propionibacterium, Proteus, Pseudomonas, Psychrobacter, Streptococcus, Streptomyces, Tetragenococcus, Weissella and Zymomonas.

36. The method of embodiment 35, wherein the cellulose-producing bacteria comprise one or more bacteria selected from Komagataeibacter xylinus, Komagataeibacter hansenii, and Komagataeibacter rhaeticus.

37. The method of any of embodiments 12-36, wherein the first culture medium comprises water, a carbon source, a nitrogen source, and nutrients.

38. The method of embodiment 37, wherein the carbon source comprises glucose and fructose.

39. The method of any of embodiments 12-38, comprising providing an acid to adjust the pH of the culture below pH 5.0.

40. The method of any of embodiments 12-38, comprising growing the cellulose-producing bacteria for four days to thirty days, for example, 10 days to 18 days.

41. The method of any of embodiments 12-40, wherein the scaffold is produced as a pellicle on a surface of the first culture medium.

42. The method of any of embodiments 12-41, wherein removing the bacterial cells from the scaffold comprises washing the scaffold with an alkaline solution, e.g., 1% NaOH, or an acidic solution, e.g., below pH 5.0 in, e.g., citric acid, at about 90° C. for about 30 minutes.

43. The method of any of embodiments 1-42, wherein the eukaryotic cells comprise fungal cells selected from Aspergillus (e.g., Aspergillus oryzae), Fusarium (e.g., Fusarium venenatum), tea fungus (e.g., Medusomyces gisevii Lindau), Geotrichum (e.g., Geotrichum candidum), Penicillium (e.g., Penicillium camemberti or Penicillium roqueforti), Neurospora (e.g., Neurospora crassa), Paecilomyces (e.g., Paecilomyces variotii) and Rhizopus ((e.g., Rhizopus oligosporus).

44. The method of embodiment 31, wherein the second culture medium comprises water, a carbon source, a nitrogen source, and nutrients.

45. The method of embodiment 44, wherein the second culture medium comprises Vogel's medium.

46. The method of any of embodiments 43 or 44, wherein the fungal cells comprise Aspergillus oryzae and the second culture medium comprises Czapek-Dox medium.

47. The method of embodiment 37 or 44, wherein the nitrogen source is present in an amount of at least 5 gms per liter, at least 7.5 grams per liter, at least 10 grams per liter or at least 15 grams per liter of the second culture medium.

48. The method of embodiment 47, wherein the nitrogen source is present in an amount of at least 5 grams per liter of the second culture medium.

49. The method of embodiment 48, wherein the nitrogen source is present in the culture medium in an amount of at least 0.5% by weight.

50. The method of any of embodiments 44-49, wherein the nitrogen source is an organic nitrogen source.

51. The method of embodiment 50, wherein the organic nitrogen source comprises amino acids, polypeptides, nucleotides or nucleic acids.

52. The method of embodiment 51, wherein the organic nitrogen source comprises a yeast extract, a peptone, or an agricultural product comprising amino acids (e.g., a hydrolyzed corn protein, a hydrolyzed soy protein, a hydrolyzed pea protein, and a corn steep liquor).

53. The method of any of embodiments 44-49, wherein the nitrogen source is an inorganic nitrogen source, e.g., a nitrate salt, a nitrite salt, an ammonium salt, a urea compound, nitrogen gas, and ammonium hydroxide.

54. The method of any of embodiments 31-42, wherein the eukaryotic cells are piscine cells and the culture medium is MEM supplemented with 10% FBS, glutamine, penicillin, and streptomycin.

55. The method of any of embodiments 31-54, wherein the second culture medium is agitated during the growing.

56. The method of any of embodiments 1-55, further comprising harvesting the composite material.

57. The method of embodiment 56, further comprising processing the composite material to alter its color, shape, flavor, texture, appearance and/or nutritional content.

58. The method of embodiment 57, wherein processing comprises one or more of:
(a) killing fungal cells in the composite material;
(b) shaping the composite material into a desired shape;
(c) adjusting the pH of the composite material;
(d) cutting the composite material;
(e) tenderizing the composite material;
(f) grinding the composite material;
(g) dicing the composite material;
(h) extruding the composite material;
(i) flavoring the composite material;
(j) coloring the composite material;
(k) adding one or more nutrients to the composite material;
(l) marinating the composite material;
(m) dehydrating the composite material;
(n) emulsifying the composite material;
(o) adding fat, oil, wax, sugar or protein to the composite material;
(p) cooking the composite material;
(q) grinding the composite material and
(r) forming layers of the composite material with a second material.

59. The method of embodiment 58, wherein, in item (l), the marinade contains yeast extract, salt, omega-3-algal oil, canthaxanthin, one or more nutrients, one or more flavorings, guar gum, trehalose, and Ca2+.

60. The method of embodiment 58, wherein processing comprises marinating the composite material in a solution comprising sugar, trimethyl acetate and a salt, and, optionally, algal oil.

61. The method of embodiment 58, wherein, in item (q), the second material is a high internal phase emulsion (HIPE).

62. The method of embodiment 61, wherein the HIPE comprises kappa carrageenan, iota carrageenan, sodium alginate, omega-3-algal oil, candelilla wax and glycerol monostearate.

63. The method of embodiment 62, wherein the HIPE further comprises a crosslinking agent.

64. The method of embodiment 63, wherein the crosslinking agent is a transglutaminase or a laccase.

65. The method of embodiment 57, wherein processing comprises killing fungal cells by boiling, high-pressure pasteurization, or exposure to ultraviolet (UV) radiation.

66. A composite material comprising:
(a) bacterial cellulose, and
(b) cellular material from a eukaryotic cell;
wherein the composite material is free or essentially free of living or dead bacterial cells.

67. The composite material of embodiment 66, comprising a scaffold comprising bacterial cellulose and voids in the scaffold, wherein eukaryotic cellular material is comprised in the voids.

68. The composite material of embodiment 66 or embodiment 67, wherein no more than 1% of DNA in the composite material encodes bacterial 16S RNA.

69. The composite material of any of embodiments 66 to 68, wherein at least a portion of the cellular material is located in voids in the scaffold.

70. The composite material of embodiments 66 to 68, wherein the cellular material comprises living cells.

71. The composite material of embodiments 66 to 68, wherein the cellular material comprises dead cells or cell residue.

72. The composite material of embodiments 66 to 68, wherein the cellular material comprises protein.

73. The composite material of embodiment 72, wherein the cellular material is cell-free.

74. The composite material of either of embodiments 72 or 73, wherein the cellular material further comprises protein.

75. The composite material of embodiment 72, wherein the protein is mycoprotein.

76. The composite material of any of embodiments 66-74, wherein the eukaryotic cell is a human cell.

77. The composite material of any of embodiments 66 to 68, wherein the eukaryotic cell is a non-human plant or animal cell.

78. The composite material of embodiment 77, wherein the eukaryotic cell is a vertebrate cell.

79. The composite material of embodiment 72, wherein the composition does not comprise a protein selected from the group consisting of a human actin, a human myosin, a human troponin, a human actinin and a human globin.

80. The composite material of embodiment 72, wherein the composition does not comprise a protein encoded by the human genome.

81. The composite material of any of embodiments 66-80, wherein the scaffold has a porosity of 1-50%.

82. The composite material of any of embodiments 66-80, wherein the material has a cutting force less than 5 kilogram-force, or less than about 3 kilogram-force, or between about 10 to about 350 psi.

83. The composite material of any of embodiments 66-80, wherein the material has a tensile strength of 150-2000 grams.

84. The composite material of any of embodiments 66-80, wherein the material has a crystallinity of 50%-70%.

85. The composite material of any of embodiments 66-80, comprising no more than any of 5%, 4%, 3%, 2%, 1% or 0.2% bacterial cells by weight.

86. The composite material of any of embodiments 66-80, comprising at least any of 5%, 6%, 7%, 8%, 9%, 10%, 15%, 20%, 25%, 30%, 35%, 40%, 45% or 50% protein by dry weight.

87. The composite material of any of embodiments 66-80, comprising no more than 5% protein by dry weight.

88. The composite material of any of embodiments 66-87, wherein the cellulose is produced by a bacterium selected from the group consisting of Komagataeibacter xylinus, Komagataeibacter hansenii, and Komagataeibacter rhaeticus.

89. The composite material of any of embodiments 66-88, further comprising one or more of a coloring agent, a flavoring agent, a supplemental nutrient, and a freeze-thaw stabilizer.

90. The composite material of embodiment 89, comprising a plurality of layers of composite material alternating with a high internal phase emulsion.

91. The composite material of embodiment 89, comprising a colorant that allows the composite material to mimic the color of tuna, salmon, yellowtail, flounder, halibut, shad, mackerel, sea bass, porgy, snapper, cod, tilapia, pollock, catfish, sardine, smelt, anchovy, eel or pangasius.

92. The composite material of embodiment 89, comprising a flavorant the provides a salty, sweet or metallic taste.

93. A composite material of any of embodiments 66-75 or 77-80, which is a food product.

94. The composite material of embodiment 93, shaped as a strip, a ring, a disk, a log, a crescent, a fan, a rectangle, a triangle, a medallion, a slab, a patty or a nugget.

95. The composite material of embodiment 93, formed as a sushi roll, e.g., composite material wrapped in rice and seaweed.

96. The composite material of embodiment 93, ground or minced and stuffed into a dumpling.

97. A method for making a composite material, comprising:
(a) culturing bacterial cells in a first culture medium to produce a scaffold of bacterial cellulose in the culture;
(b) isolating the scaffold of bacterial cellulose;
(c) culturing eukaryotic cells with the isolated scaffold in a second culture medium; and
(d) removing the second culture medium, thereby providing a composite material.

98. The method of embodiment 97, wherein the bacterial cells are cells of Komagataeibacter xylinus, Komagataeibacter hansenii, or Komagataeibacter rhaeticus.

99. The method of embodiment 97, wherein the bacterial cells are cells of Komagataeibacter xylinus, Komagataeibacter hansenii, or Komagataeibacter rhaeticus.

100. The method of embodiment 97, wherein the first culture medium is Hestrin Schram (HS) medium.

101. The method of embodiment 97, wherein the eukaryotic cells comprise plant cells or non-human animal cells.

102. The method of embodiment 97, wherein the eukaryotic cells comprise human cells.

103. The method of embodiment 97, wherein the eukaryotic cells comprise fungal cells.

104. The method of embodiment 103, wherein the fungal cells comprise Aspergillus oryzae.

105. The method of embodiment 97, wherein the second culture medium comprises Yeast Extract-Malt Extract (YM) medium.

106. The method of embodiment 97, wherein the eukaryotic cells comprise vertebrate cells.

107. The method of embodiment 106, wherein the vertebrate cells comprise cells selected from the group consisting of bovine cells, ovine cells, porcine cells, piscine cells, avian cells, shark cells, reptilian cells and amphibian cells.

108. The method of embodiment 106, wherein the vertebrate cells comprise piscine cells.

109. The method of embodiment 97, wherein the second culture medium comprises MEM supplemented with 10% FBS, glutamine, penicillin, and streptomycin.

110. The method of embodiment 97, wherein, in step (b), isolating comprises removing the culture medium from the scaffold.

111. The method of embodiment 97, wherein, in step (b), isolating comprises decellularizing the scaffold, e.g., by boiling in an alkaline solution or an acidic solution.

112. The method of embodiment 97, further comprising;
(e) killing at least some or all of the eukaryotic cells.

113. A culture comprising:
(a) bacterial cellulose;
(b) eukaryotic cells; and
(c) a culture medium;
wherein the culture is free or essentially free of living bacterial cells.

114. The culture of embodiment 113, wherein the cellulose is produced by a bacterium selected from the group consisting of Komagataeibacter xylinus, Komagataeibacter hansenii, and Komagataeibacter rhaeticus.

115. The culture of embodiment 113, wherein the eukaryotic cells comprise fungal cells.

116. The culture of embodiment 113, wherein the eukaryotic cells comprise vertebrate cells.

117. The culture of embodiment 116, wherein the vertebrate cells comprise piscine cells, molluscan cells, echinoderm cells or crustacean cells.

118. The culture of embodiment 116, wherein the vertebrate cells are not human cells.

119. An animal flesh analogue food product comprising:
(a) about 20% to about 90% dry weight of a scaffold comprising fibers of bacterial cellulose;
(b) about 0.05% to about 80% dry weight of eukaryotic protein;
(c) about 0.05% to about 5% dry weight of polyunsaturated fatty acid;
(e) a flavoring agent;
(d) a coloring agent; and, optionally,
(f) about 1% to about 80% dry weight of a supplemental nutrient; and/or
(g) a freeze-thaw stabilizer.

120. The product of embodiment 119, comprising the composite material of embodiment 66.

121. The product of embodiment 119, wherein the scaffold comprises voids having a diameter between about 0.025 microns to about 3.0 microns.

122. The product of embodiment 119, wherein at least some of the eukaryotic protein is located in voids in the scaffold.

123. The product of embodiment 119, comprising at least 5% non-fungal eukaryotic protein, e.g., protein from a metazoan or an animal.

124. The product of embodiment 119, wherein the fatty acid comprises one or more fish oils.

125. The product of embodiment 119, wherein fatty acid comprises an omega-3 polyunsaturated fatty acid, e.g., eicosapentaenoic acid and/or docosahexaenoic acid.

126. The product of embodiment 119, wherein the flavoring agent provides fish flavor.

127. The product of embodiment 119, which is a tuna analogue, and the flavoring agents provide at least any of 1, 2, 3, 4, 5, 6, 7, 8, 9, 10, 11, 12 or 13 flavors selected from metallic, savory, meaty, aldehydic, waxy, floral, lemon, citrus, marine, creamy, fatty, orris, and earthy.

128. The product of embodiment 119, which is a salmon analogue, and the flavoring agents provide at least any of 1, 2, 3, 4, 5, 6, 7, or 8 flavors selected from herbaceous, fruity, cucumber, mossy, nutty, green, creamy, and buttery.

129. The product of embodiment 119, wherein the flavoring agent and/or the supplemental nutrient comprise a yeast extract.

130. The product of embodiment 119, which is a tuna analogue, and the coloring agent provide one or more colors selected from red and yellow/orange, e.g., bright, blood red to light red, opaque and soft luster appearance.

131. The product of embodiment 119, which is a salmon analogue, and the coloring agent provide one or more colors selected from pink and orange, e.g., pinkish-orange hue with white to cream-colored fat layers.

132. The product of embodiment 119, comprising a plurality of layers of the analogue food product separated by one or more layers of a high internal phase emulsion.

133. A composite material comprising:
(a) a chitosan-alginate hydrogel, an alginate-gelatin polymer, cellulose acetate fibers, cellulose acetate-chitosan fibers, an agarose hydrogel, or an agarose-alginate hydrogel, and
(b) cellular material from a eukaryotic cell.

134. The composite material of embodiment 133, wherein the hydrogel contains voids, further wherein at least some of the eukaryotic cellular material is present in part or all of the voids.

135. The composite material of embodiment 133, wherein the cellular material comprises protein.

136. The composite material of embodiment 135, comprising at least any of 5%, 6%, 7%, 8%, 9%, 10%, 15%, 20%, 25%, 30%, 35%, 40%, 45% or 50% protein by dry weight.

137. The composite material of embodiment 133, wherein the eukaryotic cell is a fungal cell.

138. The composite material of embodiment 137, wherein the fungal cell is Aspergillus oryzae.

139. The composite material of embodiment 133, wherein the eukaryotic cell is a non-human animal cell or a plant cell.

140. The composite material of embodiment 133, further comprising one or more of a coloring agent, a flavoring agent, a supplemental nutrient, and a freeze-thaw stabilizer.

141. A method for making a composite material, the method comprising culturing fungal cells on:
(I) a chitosan-alginate hydrogel;
(II) an alginate-gelatin polymer;
(III) cellulose acetate fibers;
(IV) cellulose acetate-chitosan fibers;
(V) an agarose hydrogel; or
(VI) an agarose-alginate hydrogel.

142. The method of embodiment 141, wherein the fungal cells are cells of 10 Aspergillus oryzae.

143. A culture comprising:
(a) a chitosan-alginate hydrogel, an alginate-gelatin polymer, cellulose acetate fibers, cellulose acetate-chitosan fibers, an agarose hydrogel, or an agarose-alginate hydrogel;
(b) fungal cells; and
(c) a culture medium.

144. The culture of embodiment 143, wherein the fungal cells are cells of Aspergillus oryzae.

145. A kit comprising a product of any of embodiments 66, 93, 119, and 133; and one or more of rice, seaweed, soy sauce, wasabi and one or more chopsticks.

146. A method comprising:
(a) producing a composite material by:
   (i) co-culturing one or more bacteria and one or more fungi in a culture medium comprising a carbon source, a nitrogen source, and nutrients for time sufficient to form a pellicle at least 2.5 mm thick comprising a scaffold of bacterial cellulose and fungal protein; or
   (ii) culturing one or more bacteria for time sufficient to form a pellicle at least 2.5 mm thick comprising a scaffold of bacterial cellulose, and, optionally, killing bacteria in the pellicle; and culturing the pellicle with one or more fungi in a culture medium comprising a carbon source, a nitrogen source and nutrients for time sufficient for the fungi to infiltrate the scaffold;
(b) harvesting the composite material and treating it to kill bacterial and fungal cells, e.g., by heating in an acidic solution or an alkaline solution, e.g., at 90° C.;
(c) optionally, cutting the composite material into a plurality of pieces;
(d) marinating the composite material in a solution comprising one or more flavorings and one or more colorants.

As used herein, the following meanings apply unless otherwise specified. The words “can” and “may” are used in a permissive sense (i.e., meaning having the potential to), rather than the mandatory sense (i.e., meaning must). The words “include”, “including”, and “includes” and the like mean including, but not limited to. The singular forms “a,” “an,” and “the” include plural referents. Thus, for example, reference to “an element” includes a combination of two or more elements, notwithstanding use of other terms and phrases for one or more elements, such as “one or more.” The phrase “at least one” includes “one”, “one or more”, “one or a plurality”, and, therefore, contemplates the use of the term “a plurality”. The term “or” is, unless indicated otherwise, non-exclusive, i.e., encompassing both “and” and “or.” The term “any of” between a modifier and a sequence means that the modifier modifies each member of the sequence. So, for example, the phrase “at least any of 1, 2 or 3” means “at least 1, at least 2 or at least 3”. The term “about” refers to a range that is 5% plus or minus from a stated numerical value within the context of the particular usage. The term “consisting essentially of” refers to the inclusion of recited elements and other elements that do not materially affect the basic and novel characteristics of a claimed combination.

It should be understood that the description and the drawings are not intended to limit the invention to the particular form disclosed, but to the contrary, the intention is to cover all modifications, equivalents, and alternatives falling within the spirit and scope of the present invention as defined by the appended claims. Further modifications and alternative embodiments of various aspects of the invention will be apparent to those skilled in the art in view of this description. Accordingly, this description and the drawings are to be construed as illustrative only and are for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed or omitted, and certain features of the invention may be utilized independently, all as would be apparent to one skilled in the art after having the benefit of this description of the invention. Changes may be made in the elements described herein without departing from the spirit and scope of the invention as described in the following claims.

All publications, patents, and patent applications mentioned in this specification are herein incorporated by reference to the same extent as if each individual publication, patent, or patent application was specifically and individually indicated to be incorporated by reference.

What is claimed is:

1. A method comprising:
(a) providing a scaffold comprising bacterial cellulose; and
(b) populating the scaffold with eukaryotic cells, to produce a composite material.

2. The method of claim 1, wherein the scaffold is free or essentially free of living or dead bacterial cells.

3. The method of claim 1, wherein the eukaryotic cells comprise a fungal cell, a plant cell or an animal cell.

4. The method of claim 1, wherein the eukaryotic cells comprise an animal cell selected from a vertebrate cell, a chordate cell, an echinoderm cell, a crustacean cell, or a molluscan cell.

5. The method of claim 1, wherein the eukaryotic cells comprise a vertebrate cell selected from a primate cell, a mammalian cell, an avian cell, a reptilian cell, an amphibian cell, and a piscine cell.

6. The method of claim 1, wherein the eukaryotic cells comprise a piscine cell selected from the group consisting of tuna, salmon, yellowtail, flounder, halibut, shad, mackerel, sea bass, porgy, and snapper.

7. The method of claim 1, wherein the eukaryotic cells comprise a molluscan cell selected from the group consisting of clam, mussel, oyster, scallop, abalone, squid and octopus.

8. The method of claim 1, wherein the eukaryotic cells comprise a crustacean cell selected from the group consisting of shrimp, crab and lobster.

9. The method of claim 1, wherein the eukaryotic cells comprise a sea urchin cell.

10. The method of claim 1, wherein the eukaryotic cells comprise a human cell selected from a human stem cell, a chondrocyte, a chondroblast, a tenocyte, a tenoblast, a myoblast or a myocyte.

11. The method of claim 1, wherein the eukaryotic cells do not comprise human cells.

12. The method of claim 1, wherein (a) providing the scaffold comprises:
(i) growing cellulose-producing bacterial cells in a first culture medium to produce the scaffold comprising bacterial cellulose; and
(ii) removing the first culture medium and the bacterial cells from the scaffold.

13. The method of claim 12, comprising converting the first culture medium to a foam before growing the bacteria.

14. The method of claim 12, wherein the first culture medium further comprises a emulsifier.

15. The method of claim 13, wherein the foam is an open-cell foam.

16. The method of claim 13, wherein the foam is a closed-cell foam.

17. The method of claim 14, wherein the emulsifier is selected from one or more of a monoglyceride, a diglyceride, a polyglycerol ester such as glycerol monostearate or glycerol monooleate, lecithin, polysorbate, a phospholipid, a glycolipid, and a glycoprotein.

18. The method of claim 17, wherein the emulsifier comprises 1, 2, 3, 4, 5, 6, or 7 elements selected from:
(a) xanthan gum at a concentration of 0.2 to 2.0 weight percent;
(b) sodium alginate at a concentration of 0.2 to 2.0 weight percent;
(c) locust bean gum at a concentration of 0.2 to 2.0 weight percent;
(d) carrageenan at a concentration of 0.2 to 2.0 weight percent;
(e) guar gum at a concentration of 0.2 to 2.0 weight percent;
(f) a monoglyceride at a concentration of 0.2 to 2.0 weight percent; and
(g) a diglyceride at a concentration of 0.2 to 2.0 weight percent.

19. The method of claim 17, wherein the emulsifier comprises 1, 2, 3, 4, 5, or 6 elements selected from:
(a) xanthan gum at a concentration of 0.2 to 2.0 weight percent;
(b) gelatin at a concentration of 0.2 to 2.0 weight percent;
(c) locust bean gum at a concentration of 0.2 to 2.0 weight percent;
(d) cellulose gum at a concentration of 0.2 to 2.0 weight percent;
(e) guar gum at a concentration of 0.2 to 2.0 weight percent; and
(f) whey protein concentrate at a concentration of 0.2 to 2.0 weight percent.

20. The method of claim 14, further comprising adding a gelling agent.

21. The method of claim 20, wherein the gelling agent is selected from xanthan gum, gelatin, sodium alginate, locust bean gum, cellulose gum, carrageenan, guar gum, whey protein concentrate, dextrose, a sugar, methylcellulose, carboxymethylcellulose, and hydroxypropyl methylcellulose.

22. The method of any of claims 20-21, wherein the gelling agent comprises a monoglyceride selected from one or more of glycerol monooleate and glycerol monostearate.

23. The method of any of claims 20-21, wherein the gelling agent comprises a diglyceride selected from one or more of glyceryl distearate, glyceryl dioleate, and glyceryl dicaprylate.

24. The method of any of claims 13-23, wherein a shear force is applied to the first culture medium.

25. The method of claim 24, wherein the shear force is applied using a homogenizer, e.g., at a speed of about 1,000 rpm.

26. The method of claim 24, wherein the foam has an overrun of 150-300%.

27. The method of any of claims 24-26, wherein prior to application of the shear force, the first culture medium is inoculated with a bacterial seed inoculum of 1-10% v/v to form a bacterial culture.

28. The method of claim 24, wherein the bacterial culture is incubated at between 20°-30° C.

29. The method of claim 24, wherein, the bacterial culture is incubated at about 27° C.

30. The method of any of claims 27-29, wherein the culture is incubated for between 5 and 14 days.

31. The method of any of claims 1-30, wherein in step (b), populating the scaffold comprises growing the eukaryotic cells in a vessel comprising a second culture medium and the scaffold.

32. The method of claim 31, wherein a cell inoculum of 0.1%-85% percent of the volume of the second culture medium is introduced into the container.

33. The method of any of claims 12-32, comprising producing air into the culture medium.

34. The method of any of claims 1-33, wherein the scaffold has a porosity of 1%-50%.

35. The method of any of claims 12-34, wherein the cellulose-producing bacterial cells comprise one or more bacteria selected from Acetobacter, Bacillus, Bifidobacterium, Brachybacterium, Brevibacterium, Carnobacterium, Corynebacterium, Enterococcus, Gluconobacter, Gluconacetobacter, Corynebacterium, Halomonas, Komagataeibacter, Lactobacillus, Lactococcus, Leuconostoc, Macrococcus, Microbacterium, Micrococcus, Oenocuccus, Propionibacterium, Proteus, Pseudomonas, Psychrobacter, Streptococcus, Streptomyces, Tetragenococcus, Weissella and Zymomonas.

36. The method of claim 35, wherein the cellulose-producing bacteria comprise one or more bacteria selected from Komagataeibacter xylinus, Komagataeibacter hansenii, and Komagataeibacter rhaeticus.

37. The method of any of claims 12-36, wherein the first culture medium comprises water, a carbon source, a nitrogen source, and nutrients.

38. The method of claim 37, wherein the carbon source comprises glucose and fructose.

39. The method of any of claims 12-38, comprising providing an acid to adjust the pH of the culture below pH 5.0.

40. The method of any of claims 12-38, comprising growing the cellulose-producing bacteria for four days to thirty days, for example, 10 days to 18 days.

41. The method of any of claims 12-40, wherein the scaffold is produced as a pellicle on a surface of the first culture medium.

42. The method of any of claims 12-41, wherein removing the bacterial cells from the scaffold comprises washing the scaffold with an alkaline solution, e.g., 1% NaOH, or an acidic solution, e.g., below pH 5.0 in, e.g., citric acid, at about 90° C. for about 30 minutes.

43. The method of any of claims 1-42, wherein the eukaryotic cells comprise fungal cells selected from Aspergillus (e.g., Aspergillus oryzae), Fusarium (e.g., Fusarium venenatum), tea fungus (e.g., Medusomyces gisevii Lindau), Geotrichum (e.g., Geotrichum candidum), Penicillium (e.g., Penicillium camemberti or Penicillium roqueforti), Neurospora (e.g., Neurospora crassa), Paecilomyces (e.g., Paecilomyces variotil) and Rhizopus ((e.g., Rhizopus oligosporus).

44. The method of claim 31, wherein the second culture medium comprises water, a carbon source, a nitrogen source, and nutrients.

45. The method of claim 44, wherein the second culture medium comprises Vogel's medium.

46. The method of any of claims 43 or 44, wherein the fungal cells comprise Aspergillus oryzae and the second culture medium comprises Czapek-Dox medium.

47. The method of claim 37 or 44, wherein the nitrogen source is present in an amount of at least 5 gms per liter, at least 7.5 grams per liter, at least 10 grams per liter or at least 15 grams per liter of the second culture medium.

48. The method of claim 47, wherein the nitrogen source is present in an amount of at least 5 grams per liter of the second culture medium.

49. The method of claim 48, wherein the nitrogen source is present in the culture medium in an amount of at least 0.5% by weight.

50. The method of any of claims 44-49, wherein the nitrogen source is an organic nitrogen source.

51. The method of claim 50, wherein the organic nitrogen source comprises amino acids, polypeptides, nucleotides or nucleic acids.

52. The method of claim 51, wherein the organic nitrogen source comprises a yeast extract, a peptone, or an agricultural product comprising amino acids (e.g., a hydrolyzed corn protein, a hydrolyzed soy protein, a hydrolyzed pea protein, and a corn steep liquor).

53. The method of any of claims 44-49, wherein the nitrogen source is an inorganic nitrogen source, e.g., a nitrate salt, a nitrite salt, an ammonium salt, a urea compound, nitrogen gas, and ammonium hydroxide.

54. The method of any of claims 31-42, wherein the eukaryotic cells are piscine cells and the culture medium is MEM supplemented with 10% FBS, glutamine, penicillin, and streptomycin.

55. The method of any of claims 31-54, wherein the second culture medium is agitated during the growing.

56. The method of any of claims 1-55, further comprising harvesting the composite material.

57. The method of claim 56, further comprising processing the composite material to alter its color, shape, flavor, texture, appearance and/or nutritional content.

58. The method of claim 57, wherein processing comprises one or more of:
(a) killing fungal cells in the composite material;
(b) shaping the composite material into a desired shape;
(c) adjusting the pH of the composite material;
(d) cutting the composite material;
(e) tenderizing the composite material;
(f) grinding the composite material;
(g) dicing the composite material;
(h) extruding the composite material;
(i) flavoring the composite material;
(j) coloring the composite material;
(k) adding one or more nutrients to the composite material;
(l) marinating the composite material;
(m) dehydrating the composite material;
(n) emulsifying the composite material;
(o) adding fat, oil, wax, sugar or protein to the composite material;
(p) cooking the composite material;
(q) grinding the composite material and
(r) forming layers of the composite material with a second material.

59. The method of claim 58, wherein, in item (l), the marinade contains yeast extract, salt, omega-3-algal oil, canthaxanthin, one or more nutrients, one or more flavorings, guar gum, trehalose, and Ca²⁺.

60. The method of claim 58, wherein processing comprises marinating the composite material in a solution comprising sugar, trimethyl acetate and a salt, and, optionally, algal oil.

61. The method of claim 58, wherein, in item (q), the second material is a high internal phase emulsion (HIPE).

62. The method of claim 61, wherein the HIPE comprises kappa carrageenan, iota carrageenan, sodium alginate, omega-3-algal oil, candelilla wax and glycerol monostearate.

63. The method of claim 62, wherein the HIPE further comprises a crosslinking agent.

64. The method of claim 63, wherein the crosslinking agent is a transglutaminase or a laccase.

65. The method of claim 57, wherein processing comprises killing fungal cells by boiling, high-pressure pasteurization, or exposure to ultraviolet (UV) radiation.

66. A composite material comprising:
(a) bacterial cellulose, and
(b) cellular material from a eukaryotic cell;
wherein the composite material is free or essentially free of living or dead bacterial cells.

67. The composite material of claim 66, comprising a scaffold comprising bacterial cellulose and voids in the scaffold, wherein eukaryotic cellular material is comprised in the voids.

68. The composite material of claim 66 or claim 67, wherein no more than 1% of DNA in the composite material encodes bacterial 16S RNA.

69. The composite material of any of claims 66 to 68, wherein at least a portion of the cellular material is located in voids in the scaffold.

70. The composite material of claims 66 to 68, wherein the cellular material comprises living cells.

71. The composite material of claims 66 to 68, wherein the cellular material comprises dead cells or cell residue.

72. The composite material of claims 66 to 68, wherein the cellular material comprises protein.

73. The composite material of claim 72, wherein the cellular material is cell-free.

74. The composite material of either of claims 72 or 73, wherein the cellular material further comprises protein.

75. The composite material of claim 72, wherein the protein is mycoprotein.

76. The composite material of any of claims 66-74, wherein the eukaryotic cell is a human cell.

77. The composite material of any of claims 66 to 68, wherein the eukaryotic cell is a non-human plant or animal cell.

78. The composite material of claim 77, wherein the eukaryotic cell is a vertebrate cell.

79. The composite material of claim 72, wherein the composition does not comprise a protein selected from the group consisting of a human actin, a human myosin, a human troponin, a human actinin and a human globin.

80. The composite material of claim 72, wherein the composition does not comprise a protein encoded by the human genome.

81. The composite material of any of claims 66-80, wherein the scaffold has a porosity of 1-50%.

82. The composite material of any of claims 66-80, wherein the material has a cutting force less than 5 kilogram-force, or less than about 3 kilogram-force, or between about 10 to about 350 psi.

83. The composite material of any of claims 66-80, wherein the material has a tensile strength of 150-2000 grams.

84. The composite material of any of claims 66-80, wherein the material has a crystallinity of 50%-70%.

85. The composite material of any of claims 66-80, comprising no more than any of 5%, 4%, 3%, 2%, 1% or 0.2% bacterial cells by weight.

86. The composite material of any of claims 66-80, comprising at least any of 5%, 6%, 7%, 8%, 9%, 10%, 15%, 20%, 25%, 30%, 35%, 40%, 45% or 50% protein by dry weight.

87. The composite material of any of claims 66-80, comprising no more than 5% protein by dry weight.

88. The composite material of any of claims 66-87, wherein the cellulose is produced by a bacterium selected from the group consisting of Komagataeibacter xylinus, Komagataeibacter hansenii, and Komagataeibacter rhaeticus.

89. The composite material of any of claims 66-88, further comprising one or more of a coloring agent, a flavoring agent, a supplemental nutrient, and a freeze-thaw stabilizer.

90. The composite material of claim 89, comprising a plurality of layers of composite material alternating with a high internal phase emulsion.

91. The composite material of claim 89, comprising a colorant that allows the composite material to mimic the color of tuna, salmon, yellowtail, flounder, halibut, shad, mackerel, sea bass, porgy, snapper, cod, tilapia, pollock, catfish, sardine, smelt, anchovy, eel or pangasius.

92. The composite material of claim 89, comprising a flavorant the provides a salty, sweet or metallic taste.

93. A composite material of any of claims 66-75 or 77-80, which is a food product.

94. The composite material of claim 93, shaped as a strip, a ring, a disk, a log, a crescent, a fan, a rectangle, a triangle, a medallion, a slab, a patty or a nugget.

95. The composite material of claim 93, formed as a sushi roll, e.g., composite material wrapped in rice and seaweed.

96. The composite material of claim 93, ground or minced and stuffed into a dumpling.

97. A method for making a composite material, comprising:
(a) culturing bacterial cells in a first culture medium to produce a scaffold of bacterial cellulose in the culture;
(b) isolating the scaffold of bacterial cellulose;
(c) culturing eukaryotic cells with the isolated scaffold in a second culture medium; and
(d) removing the second culture medium, thereby providing a composite material.

98. The method of claim 97, wherein the bacterial cells are cells of Komagataeibacter xylinus, Komagataeibacter hansenii, or Komagataeibacter rhaeticus.

99. The method of claim 97, wherein the bacterial cells are cells of Komagataeibacter xylinus, Komagataeibacter hansenii, or Komagataeibacter rhaeticus.

100. The method of claim 97, wherein the first culture medium is Hestrin Schram (HS) medium.

101. The method of claim 97, wherein the eukaryotic cells comprise plant cells or non-human animal cells.

102. The method of claim 97, wherein the eukaryotic cells comprise human cells.

103. The method of claim 97, wherein the eukaryotic cells comprise fungal cells.

104. The method of claim 103, wherein the fungal cells comprise Aspergillus oryzae.

105. The method of claim 97, wherein the second culture medium comprises Yeast Extract-Malt Extract (YM) medium.

106. The method of claim 97, wherein the eukaryotic cells comprise vertebrate cells.

107. The method of claim 106, wherein the vertebrate cells comprise cells selected from the group consisting of bovine cells, ovine cells, porcine cells, piscine cells, avian cells, shark cells, reptilian cells and amphibian cells.

108. The method of claim 106, wherein the vertebrate cells comprise piscine cells.

109. The method of claim 97, wherein the second culture medium comprises MEM supplemented with 10% FBS, glutamine, penicillin, and streptomycin.

110. The method of claim 97, wherein, in step (b), isolating comprises removing the culture medium from the scaffold.

111. The method of claim 97, wherein, in step (b), isolating comprises decellularizing the scaffold, e.g., by boiling in an alkaline solution or an acidic solution.

112. The method of claim 97, further comprising;
(e) killing at least some or all of the eukaryotic cells.

113. A culture comprising:
(a) bacterial cellulose;
(b) eukaryotic cells; and
(c) a culture medium;
wherein the culture is free or essentially free of living bacterial cells.

114. The culture of claim 113, wherein the cellulose is produced by a bacterium selected from the group consisting of Komagataeibacter xylinus, Komagataeibacter hansenii, and Komagataeibacter rhaeticus.

115. The culture of claim 113, wherein the eukaryotic cells comprise fungal cells.

116. The culture of claim 113, wherein the eukaryotic cells comprise vertebrate cells.

117. The culture of claim 116, wherein the vertebrate cells comprise piscine cells, molluscan cells, echinoderm cells or crustacean cells.

118. The culture of claim 116, wherein the vertebrate cells are not human cells.

119. An animal flesh analogue food product comprising:
(a) about 20% to about 90% dry weight of a scaffold comprising fibers of bacterial cellulose;
(b) about 0.05% to about 80% dry weight of eukaryotic protein;
(c) about 0.05% to about 5% dry weight of polyunsaturated fatty acid;
(e) a flavoring agent;
(d) a coloring agent; and, optionally,
(f) about 1% to about 80% dry weight of a supplemental nutrient; and/or
(g) a freeze-thaw stabilizer.

120. The product of claim 119, comprising the composite material of claim 66.

121. The product of claim 119, wherein the scaffold comprises voids having a diameter between about 0.025 microns to about 3.0 microns.

122. The product of claim 119, wherein at least some of the eukaryotic protein is located in voids in the scaffold.

123. The product of claim 119, comprising at least 5% non-fungal eukaryotic protein, e.g., protein from a metazoan or an animal.

124. The product of claim 119, wherein the fatty acid comprises one or more fish oils.

125. The product of claim 119, wherein fatty acid comprises an omega-3 polyunsaturated fatty acid, e.g., eicosapentaenoic acid and/or docosahexaenoic acid.

126. The product of claim 119, wherein the flavoring agent provides fish flavor.

127. The product of claim 119, which is a tuna analogue, and the flavoring agents provide at least any of 1, 2, 3, 4, 5, 6, 7, 8, 9, 10, 11, 12 or 13 flavors selected from metallic, savory, meaty, aldehydic, waxy, floral, lemon, citrus, marine, creamy, fatty, orris, and earthy.

128. The product of claim 119, which is a salmon analogue, and the flavoring agents provide at least any of 1, 2, 3, 4, 5, 6, 7, or 8 flavors selected from herbaceous, fruity, cucumber, mossy, nutty, green, creamy, and buttery.

129. The product of claim 119, wherein the flavoring agent and/or the supplemental nutrient comprise a yeast extract.

130. The product of claim 119, which is a tuna analogue, and the coloring agent provide one or more colors selected from red and yellow/orange, e.g., bright, blood red to light red, opaque and soft luster appearance.

131. The product of claim 119, which is a salmon analogue, and the coloring agent provide one or more colors selected from pink and orange, e.g., pinkish-orange hue with white to cream-colored fat layers.

132. The product of claim 119, comprising a plurality of layers of the analogue food product separated by one or more layers of a high internal phase emulsion.

133. A composite material comprising:
(a) a chitosan-alginate hydrogel, an alginate-gelatin polymer, cellulose acetate fibers, cellulose acetate-chitosan fibers, an agarose hydrogel, or an agarose-alginate hydrogel, and
(b) cellular material from a eukaryotic cell.

134. The composite material of claim 133, wherein the hydrogel contains voids, further wherein at least some of the eukaryotic cellular material is present in part or all of the voids.

135. The composite material of claim 133, wherein the cellular material comprises protein.

136. The composite material of claim 135, comprising at least any of 5%, 6%, 7%, 8%, 9%, 10%, 15%, 20%, 25%, 30%, 35%, 40%, 45% or 50% protein by dry weight.

137. The composite material of claim 133, wherein the eukaryotic cell is a fungal cell.

138. The composite material of claim 137, wherein the fungal cell is Aspergillus oryzae.

139. The composite material of claim 133, wherein the eukaryotic cell is a non-human animal cell or a plant cell.

140. The composite material of claim 133, further comprising one or more of a coloring agent, a flavoring agent, a supplemental nutrient, and a freeze-thaw stabilizer.

141. A method for making a composite material, the method comprising culturing fungal cells on:
(I) a chitosan-alginate hydrogel;
(II) an alginate-gelatin polymer;
(Ill) cellulose acetate fibers;
(IV) cellulose acetate-chitosan fibers;
(V) an agarose hydrogel; or
(VI) an agarose-alginate hydrogel.

142. The method of claim 141, wherein the fungal cells are cells of Aspergillus oryzae.

143. A culture comprising:
(a) a chitosan-alginate hydrogel, an alginate-gelatin polymer, cellulose acetate fibers, cellulose acetate-chitosan fibers, an agarose hydrogel, or an agarose-alginate hydrogel;
(b) fungal cells; and
(c) a culture medium.

144. The culture of claim 143, wherein the fungal cells are cells of Aspergillus oryzae.

145. A kit comprising a product of any of claims 66, 93, 119, and 133; and one or more of rice, seaweed, soy sauce, wasabi and one or more chopsticks.

146. A method comprising:
(a) producing a composite material by:
(i) co-culturing one or more bacteria and one or more fungi in a culture medium comprising a carbon source, a nitrogen source, and nutrients for time sufficient to form a pellicle at least 2.5 mm thick comprising a scaffold of bacterial cellulose and fungal protein; or
(ii) culturing one or more bacteria for time sufficient to form a pellicle at least 2.5 mm thick comprising a scaffold of bacterial cellulose, and, optionally, killing bacteria in the pellicle; and culturing the pellicle with one or more fungi in a culture medium comprising a carbon source, a nitrogen source and nutrients for time sufficient for the fungi to infiltrate the scaffold;
(b) harvesting the composite material and treating it to kill bacterial and fungal cells, e.g., by heating in an acidic solution or an alkaline solution, e.g., at 90° C.;
(c) optionally, cutting the composite material into a plurality of pieces;
(d) marinating the composite material in a solution comprising one or more flavorings and one or more colorants.
